(19) United States
(12) Patent Application Publication
(10) Pub. No.: US 2025/0287930 A1
(43) Pub. Date: Sep. 18, 2025

(54) REGULATION OF IMMUNE RESPONSES THROUGH SELECTIVE NEURAL ACTIVATION

(71) Applicant: The Trustees of Columbia University in the City of New York, New York, NY (US)

(72) Inventors: Charles S. Zuker, New York, NY (US); Hao Jin, New York, NY (US)

(21) Appl. No.: 19/083,126

(22) Filed: Mar. 18, 2025

Related U.S. Application Data
(60) Provisional application No. 63/566,738, filed on Mar. 18, 2024.

(51) Int. Cl. A01K 67/0276

(52) U.S. Cl. CPC A01K 67/0276; A01K 2217/075; A01K 2227/105; A01K 2267/0325

(57) ABSTRACT

Disclosed herein are methods for regulating an immune response using selective activation of neurons in the caudal nucleus of the solitary tract.

### CROSS REFERENCE TO RELATED APPLICATIONS

This application claims the benefit of U.S. Provisional Application No. 63/566,738, filed Mar. 18, 2024, the content of which is herein incorporated by reference in its entirety.

### TECHNICAL FIELD

Disclosed herein are methods of regulating the immune response.

### BACKGROUND OF THE INVENTION

Immune responses play critical roles in the body's ability to maintain health and fight disease. Anatomically, there are extensive bidirectional connections between the brain and the immune system suggesting that the brain could monitor and modulate peripherical immune activity. However, the functional meaning of these connections in the neuronal control of immune response remains largely unknown. Regulation of the immune response is a central strategy to treat disease and injury, making novel methods to control the immune response essential to the advancement of health and medicine.

### SUMMARY OF THE INVENTION

Disclosed herein are methods of regulating an immune response in a mammal comprising modulating the activity of one or more TRPA1-expressing neurons in a vagal ganglion of the mammal to thereby regulate the immune response, wherein, when the neuron is engineered to express a designer receptor exclusively activated by a designer drug (DREADD), there is a decreased serum level of one or more proinflammatory cytokines and/or an increased serum level of one or more anti-inflammatory cytokines in the presence of a DREADD ligand.

Disclosed herein are methods of regulating an immune response in a mammal comprising modulating the activity of one or more CGRP receptor-expressing neurons in a vagal ganglion of the mammal to thereby regulate the immune response, wherein, when the neuron is engineered to express a DREADD, there is an increased serum level of one or more proinflammatory cytokines and/or a decreased serum level of one or more anti-inflammatory cytokines in the presence of a DREADD ligand.

Disclosed herein are methods of regulating an immune response in a mammal comprising modulating the activity of one or more neurons in the brainstem of the mammal, wherein, the neuron is activated by lipopolysaccharide (LPS) to induce release of pro-inflammatory cytokines, and wherein, when the neuron is engineered to express a DREADD, there is a reduced LPS-evoked release of pro-inflammatory cytokines and/or increased release of anti-inflammatory cytokines in the presence of a DREADD ligand.

Disclosed herein are methods of assessing the ability of a stimulus to regulate an immune response in a subject. In some embodiments, the methods comprise applying the stimulus (e.g., administration of a therapeutic agent) to the subject. In some embodiments, the method comprises measuring in the subject, in response to the stimulus, activation of glutamatergic neurons. In some embodiments, the method comprises measuring in the subject, in response to the stimulus, activation of dopamine beta-hydroxylase (Dbh)-positive neurons. In some embodiments, the method comprises identifying the stimulus as capable of regulating the immune response based on the activation of the Dbh-positive neurons.

Disclosed herein are model animals for screening the ability of a stimulus to regulate an immune response. In some embodiments, the model animals comprise a genetically encoded calcium indicator expressed in glutamatergic neurons of the animal. In some embodiments, the model animals comprise a genetically encoded calcium indicator expressed in Dbh-positive neurons of the animal.

Disclosed herein are methods of regulating an immune response in a subject in need thereof. In some embodiments, the methods comprise applying a stimulus to the subject capable of selectively activating glutamatergic neurons in the subject's caudal nucleus of the solitary tract. In some embodiments, activation of the glutamatergic neurons (e.g., Dbh-positive neurons) suppresses an immune response in the subject.

### BRIEF DESCRIPTION OF THE DRAWINGS

The summary, as well as the following detailed description, is further understood when read in conjunction with the appended drawings. For the purpose of illustrating the claimed methods, there are shown in the drawings exemplary embodiments of the methods; however, the methods are not limited to the specific methods disclosed. In addition, the drawings are not necessarily drawn to scale.

FIG. 1 illustrates the cellular components of the innate and adaptive immune response. Upon detection of an infectious agent, the immune system triggers and innate response followed by adaptive response. These two responses are executed by various cellular components as depicted and need to be tightly regulated to effectively combat an infection.

FIG. 2 depicts the central role of the brain in controlling immune responses. The brain plays an essential role in regulating the immune response although the exact regions that control and respond to immune activation were previously not well understood.

FIG. 3 illustrates the anatomical connection between the nervous system and the immune system. Anatomically there are extensive connections between the brain and the immune system. However, the functional significance of these connections in the neuronal control of immune response remains largely unknown.

FIG. 4 depicts the effect of learning and internal states on the immune response. The brain detects peripheral immune activity and regulates peripheral immune response. Further, learning and internal states can regulate the immune response.

FIG. 5 shows a schematic of LPS stimulation strategy for Fos Induction and illustrates neuron activation in the caudal nucleus of the solitary tract upon immune system stimulation. Peripheral immune challenge evokes brain activities. LPS was used to induce an innate immune response and the brain was examined for the induction of c-fos as a proxy for neural activity. Strong Fos induction is observed in the caudal nucleus of the solitary tract (cNST, highlighted) in response to LPS stimulus but not control stimulus. When the body experiences bacterial infections, the bacterial membrane component lipopolysaccharide (LPS) triggers strong innate immune response. LPS (right) but not saline control (left) strongly activates cNST (circled area). For each condition, a representative image from N>=3 animals is shown. Scale bar: 100 μm. Strong bilateral c-fos labeling was detected in the caudal nucleus of the solitary tract (cNST) in the brainstem.

FIG. 6 depicts the role of the vagus nerve in transmitting signals between the caudal nucleus of the solitary tract and the peripheral immune response. cNST is a central hub of interception receiving body-wide physiological signals via the vagus nerve. Monitoring the vagal response to immune signals at this gateway to brain provides an understanding of how the brain monitors peripheral immune activities.

FIG. 7 illustrates a subset of neurons in the nodose ganglion that respond to pro-inflammatory cytokines. Calcium responses in vagal sensory neurons expressing the genetically encoded fluorescent calcium indicator GCaMP6s were imaged while administering cytokines intraperitoneally. The platform was modified to image the response of vagal sensory neurons to cytokines. The cytokine was administered via intraperitoneal injection and calcium activity in the nodose ganglion was monitored. Heat maps depicting z-score-normalized fluorescence traces from vagal neurons identified as responders of proinflammatory cytokine (TNFa). Neural activity was detected using an in-vivo calcium imaging platform with genetically encoded calcium indicator GCaMP6 expressed in all vagal sensory neurons. Each trial represents the activity of a single cell. Vertical dashed lines indicate the time of cytokine administration (intraperitoneal). Note the absence of response to vehicle control (Saline, 0.9% NaCl). Administration of pro-inflammatory cytokine triggered robust activity in a unique subset of vagal sensory neurons that are not activated by control stimuli like glucose or saline.

FIG. 8 illustrates a subset of neurons in the nodose ganglion that respond to anti-inflammatory cytokines. Heat maps depicting z-score-normalized fluorescence traces from vagal neurons identified as responders of anti-inflammatory cytokines (IL-4). Each row represents the activity of a single cell. Vertical dashed lines indicate the time of cytokine administration (intraperitoneal). Note the absence of response to vehicle control (Saline, 0.9% NaCl). Neural activity was detected using an in-vivo calcium imaging platform with genetically encoded calcium indicator GCaMP6 expressed in all vagal sensory neurons. The platform was modified to image the response of vagal sensory neurons to cytokines. The cytokine was administered via intraperitoneal injection and calcium activity in the nodose ganglion was monitored. Administration of anti-inflammatory cytokine triggered robust activity in a unique subset of vagal sensory neurons that are not activated by control stimuli like glucose or saline.

FIG. 9 illustrates the mapping of the receptor for pro-inflammatory cytokines. Heat maps depicting z-score-normalized calcium responses of VIP expressing vagal neurons to pro-inflammatory cytokine stimulation (bath application for 3 mins). Each trial represents the activity of a single cell. Dashed lines indicate stimulation time window. Approximately 30% of VIP neurons respond. The receptor for pro-inflammatory cytokine was mapped to a small cluster by exploiting the single cell atlas of vagal sensory neurons. Using the cre driver to express GCaMP in this cluster, this cluster enriches neurons that are activated by pro-inflammatory cytokine.

FIG. 10 depicts the relationship between the caudal nucleus of the solitary tract in regulating the peripheral immune response. The brain uses information conveyed by vagal sensory neurons to positively and negatively feedback modulate immune response.

FIG. 11 shows a schematic of TRAP strategy to mark LPS-responsive neurons in the cNST (highlighted) with a fluorescent reporter (tdTomato) and illustrates neuron activation in the caudal nucleus of the solitary tract upon immune system stimulation using an activity based labeling strategy. The activity based labelling strategy, TRAP, was used to identify and access neuronal populations activated during LPS induced inflammation. Neurons TRAPed by LPS (right) but not saline control (left) are seen in the cNST (circled area). For each condition, a representative image from N>=3 animals is shown. Scale bar: 500 μm. Strong labelling with a florescence reporter is observed in the caudal nucleus of the solitary tract, the central target of vagal afferents, in LPS-treated animals but not saline treated animals.

FIG. 12 illustrates the effect of neuronal stimulation in the caudal nucleus of the solitary tract on the immune response. Chemogenetic activation of the LPS-TRAPed population in the cNST strongly inhibited the LPS evoked release of pro-inflammatory cytokine TNFa and increased that anti-inflammatory cytokine IL-10. Neuronal populations were chemogenetically activated with designer receptor exclusively activated by a designer drug (DREADD) while the animal is given LPS and cytokine levels in the blood were measured. In the control animals, LPS induced the strong release of pro-inflammatory TNFa, reaching 200 pg/ml in the peripheral blood. When this brainstem neuronal population was activated with DREADD, there was a remarkable suppression of pro-inflammatory cytokine release, resulting in 90% reduction in the TNFa in the blood. The same results were obtained for another proinflammatory cytokine, IL-6. In addition to suppressing the release of pro-inflammatory cytokines, activation of this neuronal population markedly increase the release of the anti-inflammatory cytokine, IL-10. Thus, this brain circuit triggers a strong anti-inflammatory response while suppressing pro-inflammatory cytokines. Note that under normal conditions (control) the immune insult triggers a very large induction of pro-inflammatory cytokines (TNFa; left panels), and this induction is suppressed by activation of the cNST neurons (compare bars). In addition, activation of these same neurons triggers the strong induction of peripheral anti-inflammatory cytokines (IL-10; right panels) (compare bars).

FIG. 13 illustrates a single cell atlas that maps neurons responsive to immune activation into a specific cluster. Using a single cell atlas of cNST, LPS responsive neurons map to a small cluster, cNST1.

FIG. 14 depicts the bidirectional relationship between activation of neurons in the caudal nucleus of the solitary tract and regulation of the peripheral immune response. Identification of immune enhancing circuits within the brain. Immune responsive vagal and cNST neurons regulate peripheral immune information.

FIG. 15 illustrates the effect of learning on an adaptive immune response. Learning can profoundly influence peripheral immune function such that an anticipatory cue through learning can potentiate the immune response. In an immune-conditioning paradigm, mice were given an antigenic stimulus, ovalbumin, in the presence of grape water for three consecutive days while in the control group, grape water was given four days before ovalbumin administration. Significantly higher antibody titer was observed following re-exposure to ovalbumin and grape odor in the test group compared to the control, suggesting that grape odor is effectively conditioned mice to enhance immune response.

FIG. 16 depicts the relationship between olfactory cues and adaptive immune system regulation. The brain associates the immune signal with the sensory cue (grape odor, the conditioned stimulus) to impart the otherwise neutral olfactory cue with an immune modulating capacity.

FIG. 17 depicts the relationship between neuronal activation, learned activity, and immune system regulation. The vagal-brainstem circuit reports and regulates peripheral immune responses. Vagal sensory neurons contain distinct subsets that respond to pro- and anti-inflammatory cytokines. Activating brainstem neurons receiving vagal input dramatically alters the nature of innate immune response. An immune-conditioning paradigm shows that predictive cues can impact immune responses.

FIG. 18 shows that silencing cNST-responsive neurons de-suppresses inflammatory responses. Through utilization of genetic trapping and introduction of a chemogenetic silencer, the effect of silencing on cNST neurons in the emergence of LPS-induced inflammatory responses was investigated. When LPS-activated cNST neurons are silenced, there is a complete change in the inflammatory response: an out-of-control increase in the release of proinflammatory cytokines and a dramatic dampening in the release of anti-inflammatory cytokines.

FIG. 19 illustrates that activating cNST-responsive neurons dramatically suppresses inflammatory responses. Though utilization of the TRAP system and selective chemogenetic activation, the effect after LPS injection on the neurons was examined. These neurons were artificially activated after LPS using a synthetic drug, CNO, and then cytokine levels in the circulation were analyzed in response to LPS. Activating these neurons leads to a dramatic suppression of the release of pro-inflammatory cytokines, accompanied with a very large increase in the levels of anti-inflammatory cytokines.

FIG. 20 shows imaging of vagal sensory neuron responses to pro-& anti-inflammatory cytokines. There is a selective population of neurons that responds to pro-inflammatory cytokines, and a separate population that responds to anti-inflammatory cytokines.

FIG. 21 shows genetic identification of vagal neurons responding to pro-inflammatory signals. A single cell atlas of the vagal ganglia (Nodose), combined with functional imaging demonstrate that TrpA1-expressing neurons in the vagal ganglia respond to anti-inflammatory signals.

FIG. 22 illustrates that vagal neurons mediate the body-brain immune modulation. Artificial activation of TRPA1-expressing neuron in the vagal ganglia (chemogenetically in this example) mimics the effect of activating the cNST neurons and leads to the suppression of pro-inflammatory cytokines, and the induction of anti-inflammatory cytokines.

FIG. 23 shows the genetic identification of vagal neurons responding to pro-inflammatory signals. Single cell atlas of the vagal ganglia (Nodose), combined with functional imaging demonstrate that the Calca-expressing (calcitonin gene-related peptide—CGRP) neurons in the vagal ganglia carry TNFa pro-inflammatory signals from the body to the brain.

FIG. 24 illustrates that an immune insult with LPS triggers strong innate immune responses, characterized by the release of proinflammatory cytokines like IL-1b, IL-6, and TNFa, and the release of anti-inflammatory cytokines that help modulate and dampen the inflammatory response, like IL-10. A balanced ratio of pro- and anti-inflammatory cytokines is important for an appropriate immune response.

FIG. 25 shows that activating the cNST Crhr2 neurons is sufficient to suppress immune response/inflammation.

FIG. 26 shows the innate immune responses activate the brain via the vagal-brain axis. Panel a, Left, cartoon illustrating the time course of LPS-induced cytokine measurements. Wild type mice were injected with control saline or LPS intraperitoneally, and peripheral blood was taken every 2 hours (0, 2, 4 and 6 h) to measure circulating levels of pro-inflammatory (IL-6, IL-1β, TNF-α), and anti-inflammatory (IL-10) cytokines by ELISA, right. Red/green circles, LPS-injected mice, black circles, saline injected animals; n=5 mice. Values are means±SEM. Panel b, Left, schematic of Fos induction by LPS stimulation. Mice received an intraperitoneal injection of saline or LPS, and two hours later, brains were extracted, sliced and immunostained for Fos expression. Strong bilateral Fos labeling is detected in neurons of the cNST (highlighted in yellow) in LPS—but not in saline-stimulated mice; n=5 mice. Right, quantification of Fos-positive neurons. The equivalent area of the cNST (200 μm×200 μm, bregma −7.5 mm) was processed, and positive neurons were counted for each stimuli. Values are means±SEM; Mann-Whitney U-tests, p=0.008. Scale bar, 200 μm. AP, Area Postrema; DMV; Dorsal Motor Vagal Complex. Panel c, Left, schematic illustrating fiber photometry experiments to monitor LPS-evoked activity in the cNST. AAV viruses carrying a Cre-dependent GCaMP6s construct were targeted to the cNST of Vglut2-cre mice for fiber photometry recording (see FIG. 38E). Middle, neural responses following LPS (dark blue traces, 0.5 mg kg-1, n=6; light blue traces, 0.1 mg kg-1, n=4) or control saline (black traces, n=6) stimulation. The solid trace is the mean and the shaded area represents SEM. Shown in orange are responses after bilateral vagotomy (n=6). The saline and LPS injections were done as successive stimulations in the same animals. Scale bar: ΔF/F. The red arrow indicates the time of the injections. Right panel: quantification of responses. AUC: area under the curve. LPS dose=0.5 mg kg-1. Values are means±SEM; Wilcoxon test (Saline vs LPS), p=0.03; Mann-Whitney U-test (LPS vs Vagotomy), p=0.004; Mann-Whitney U-test (Saline vs Vagotomy), p=0.18. Note the severe loss of responses following removal of the vagal communication pathway (average response is reduced ˜80%; AUC: 52.6±7.2 to AUC: 10.6±6.1).

FIGS. 27A-C show that removing brain regulation transforms the inflammatory response. FIG. 27A, Neurons marked by the Cre-dependent reporter after LPS-TRAPing (red, tdTomato) are the same as the Fos-positive neurons labeled after a second cycle of LPS treatment (green). By comparing the number of neurons expressing the fluorescent tdTomato reporter to the number of neurons labelled by the Fos antibodies, it was determined that more than 80% of LPS-TRAPed neurons were also positive for LPS-Fos (n=4). Scale bar, 50 μm. FIG. 27B, Inhibition of LPS-activated neurons in the cNST greatly increases the inflammatory response. AAV viruses carrying a control mCherry construct, or the hM4Di inhibitory DREADD, were targeted to the cNST of LPS-TRAP2 mice for chemogenetic silencing. While control animals injected with LPS exhibit the expected induction of pro- and anti-inflammatory cytokines (compare mCherry Saline vs mCherry LPS injected mice; grey bars). Animals with silenced cNST neurons displayed extraordinary increases in pro-inflammatory cytokines (IL-6 and IL-1β, compare red and grey bars), and a concomitant large reduction in the levels of anti-inflammatory (IL-10, compare green and grey bars). All mice, experimental and controls, were given CNO 1 h before either the saline or LPS injection; n=6 for each group. Values are means±SEM; Mann-Whitney U-tests, p=0.24 (Saline, IL-6), p=0.97 (Saline, IL-1β), p=0.78 (Saline, IL-10); p=0.004 (LPS, IL-6), p=0.004 (LPS, IL-1β), p=0.002 (LPS, IL-10). FIG. 27C, Chemogenetic activation of the cNST neurons during an immune response suppresses inflammation. Shown are levels of anti-inflammatory (IL-10) and pro-inflammatory (IL-6, IL-1β) cytokines in the peripheral blood of mice expressing excitatory DREADD (hM3Dq), or control mCherry, in the LPS-activated cNST neurons. All animals, experimental and controls, were given CNO 1 h before either the saline or LPS injection (n=6 for each group). Values are means±SEM; Mann-Whitney U-tests, p=0.17 (Saline, IL-6), p=0.93 (Saline, IL-1β), p=0.37 (Saline, IL-10); p=0.002 (LPS, IL-6), p=0.002 (LPS, IL-1β), p=0.002 (LPS, IL-10). Note the vast increase in the levels of anti-inflammatory, and large decrease in the levels of pro-inflammatory cytokines.

FIG. 28 shows that a genetically defined population of cNST neurons modulates body immunity. Panel a, Single-cell RNA sequencing (scRNA-seq) cataloging neuronal clusters in the cNST. A uniform manifold approximation and projection (UMAP) plot of transcriptomic data reveals 14 Glutamatergic neuronal clusters (1-14, colored) and 6 GABAergic clusters (15-20, grey). Panel b, scRNA-seq of individual LPS-TRAPed neurons from the cNST. The tdTomato-labeled LPS-TRAPed cells were isolated by FACS and individually sequenced. The UMAP of LPS-TRAPed neurons was then superimposed to the UMAP of the entire cNST map, showing that the LPS-TRAPed neurons (highlighted in red) are found primarily in excitatory clusters (7, 10, 12). Panel c, UMAP plot showing the normalized expression of Dbh gene, and the strategy for chemogenetic activation of the DBH-expressing cNST neurons. An AAV virus carrying the excitatory Cre-dependent hM3Dq DREADD (AAV-DIO-hM3Dq) was targeted bilaterally to the cNST of Dbh-cre mice. Panel d, Chemogenetic activation of DBH cNST neurons suppresses inflammation. Shown are levels of anti-inflammatory (IL-10) and pro-inflammatory (IL-6, IL-1β) cytokines in the peripheral blood of mice expressing either excitatory DREADD (hM3Dq) or control mCherry, 2 hours after LPS stimulation. All mice were given CNO 1 hr prior to saline or LPS injection. n=4 animals for each group. Note the major decrease in pro-inflammatory cytokines, and large increase in anti-inflammatory IL-10. Values are means±SEM; Mann-Whitney U-tests, p=0.08 (Saline, IL-6), p=0.20 (Saline, IL-1β), p=0.23 (Saline, TNF-α), p=0.77 (Saline, IL-10); p=0.03 (LPS, IL-6), p=0.03 (LPS, IL-1β), p=0.03 (LPS, TNF-α), p=0.03 (LPS, IL-10).

FIG. 29 shows that vagal neurons respond to anti- and pro-inflammatory cytokines. Panel a, Recording calcium responses in vagal sensory neurons expressing the fluorescent calcium indicator GCaMP6s while stimulating mice with cytokines intraperitoneally. The heat maps depict z-score-normalized fluorescence traces from two non-overlapping populations of neurons: responders to pro-inflammatory (Pro) cytokines (upper panels) and responders to anti-inflammatory (Anti) cytokines (middle panels). Each row represents the activity of a single cell over 5 mins. Stimulus was given at 60 seconds (indicated by the dashed line). n=5 mice, TNF-α (3 mice), IL-1β (2 mice), IL-10 (5 mice). As a positive control, intestinal stimulation with glucose (Glu, 10 s) was used; this activates the gut-brain axis, but stimulates a completely different non-overlapping population of vagal neurons (lower panels). Note that these imaging experiments used cytokine concentrations that were either lower or comparable to that measured during LPS-induced inflammation (See also, FIG. 44). In these experiments, 21 of 423 imaged vagal sensory neurons responded to Pro-inflammatory stimuli (13 to TNF-α, and 8 to IL-1β), and 11/423 responded to anti-inflammatory cytokines; the overall percent of responding neurons is similar to what is observed for vagal neurons dedicated to other body-brain signaling pathways, like sugar-preference and control of breathing. Panel b, Vagal neurons are not directly activated by LPS, even when using high concentrations of LPS (0.5 mg kg-1; n=5 mice; pro: TNF-α; anti: IL-10). Panel c, Similar experiments were carried out by using a perfusion chamber rather than IP injections of LPS and control stimuli. The heat maps depict z-score-normalized fluorescence traces from the vagal neurons responding to pro-inflammatory versus anti-inflammatory cytokines. Each row represents the averaged activity of a single cell to 2 trials. Dashed lines denote stimulus time window (180 sec). n=19 mice; IL-1β (7 mice), IL-6 (12 mice), IL-10 (19 mice). See also FIGS. 41 and 42.

FIG. 30 shows vagal control of inflammation. Panel a, Strategy for chemogenetic activation of TRPA1 vagal neurons. An excitatory DREADD receptor (via AAV-DIO-hM3Dq) was targeted bilaterally to the nodose ganglion of Trpa1-cre mice6. Control animals received injection of AAV-DIO-mCherry. Panel b, Chemogenetic activation of TRPA1 vagal neurons suppresses inflammation. The graphs show levels of pro-inflammatory (IL-6, IL-1β) and anti-inflammatory (IL-10) cytokines in the peripheral blood of the mice expressing the excitatory DREADD (hM3Dq; n=7 mice) and control (mCherry; n=4 mice). Blood samples were collected 2 hours after LPS stimulation, and all animals were given CNO 1 hr prior to LPS injection. Values are means±SEM; Mann-Whitney U-tests, p<0.01 (IL-6), p<0.01 (IL-1β), p<0.01 (IL-10). Panel c, The heat maps depict z-score-normalized fluorescence traces from IL-10 responding TRPA1 vagal neurons. Each row represents the activity of a single cell over 15 mins. The experiment was carried out using IP or perfusion with similar results. n=6 mice. Pro, IL-6, Anti, IL-10. Note selective responses to anti-, but not pro-inflammatory stimuli. A total of 27 of 189 imaged TRPA1 neurons responded to IL-10. Panel d, Strategy for chemogenetic activation of CALCA vagal neurons. An excitatory DREADD receptor (via AAV-DIO-hM3Dq) was targeted bilaterally to the nodose ganglion of Calca-cre mice. Control animals received injection of AAV-DIO-mCherry. Panel e, Chemogenetic activation of CALCA vagal neurons reduces levels of pro-inflammatory cytokines. The graphs show levels of anti-inflammatory (IL-10) and pro-inflammatory cytokines (IL-6, IL-1β) in the peripheral blood of the mice expressing the excitatory DREADD (hM3Dq; n=11 mice) and control (mCherry; n=9 mice). Blood samples were collected 2 hours after LPS stimulation, and all animals were given CNO 1 hr prior to LPS injection. Values are means±SEM; Mann-Whitney U-tests, p<0.01 (IL-6), p=0.001 (IL-1β), p=0.88 (IL-10). Panel f, The heat maps depict z-score-normalized fluorescence traces from pro-inflammatory (IL-6, IL-1β) responding CALCA vagal neurons. Each row represents the activity of a single cell over 15 mins. The experiment was carried out using IP. n=6 mice. Note selective responses to pro-, but not anti-inflammatory stimuli. A total of 35 of 211 imaged CALCA neurons responded to the pro-inflammatory cytokine stimuli.

FIG. 31 shows vagal-brain restoration of immune balance. Panel a, Strategy for chemogenetic activation of TRPA1 vagal neurons, and DBH cNST neurons. An excitatory DREADD receptor (via AAV-DIO-hM3Dq) was targeted bilaterally to the nodose ganglion of Trpa1-cre mice, and to the cNST of Dbh-cre mice. As controls, mice from the same cre-driver lines received injections of AAV-DIO-mCherry. Mice were challenged with a lethal dose of LPS (12.5 mg kg-1), and the TRPA1 vagal neurons, or the DBH cNST neurons, were activated by injection of CNO at 6 h intervals beginning 1 h prior to LPS injection, during the first day of the experiment (3 injections over 18 hrs). Panel b, Activation of TRPA1 vagal neurons or DBH-expressing cNST neurons rescue animals from LPS-induced sepsis. The graphs show survival curves. The control animals, injected with mCherry, were subjected to the same regime of CNO injections. Vagal activation, n=9 for control mCherry (black lines), and n=8 for hM3Dq animals (green lines); cNST activation, n=9 mice for each group. Log-rank (Mantel-Cox) tests, p<0.001 (vagal), p<0.001 (cNST). Red arrows indicate the injections of LPS. Note that all control mice, in both groups, die within the first 4 days. Panel c, Diagram of DSS-induced ulcerative colitis. Panel d, Activation of TRPA1 vagal neurons protect animals from DSS-induced colon damage. An excitatory DREADD receptor (via AAV-DIO-hM3Dq) was targeted bilaterally to the nodose ganglion of Trpa1-cre mice. As controls, mice from the same cre-driver lines received injections of AAV-DIO-mCherry. Experimental and control animals were provided with CNO in the drinking water (see Methods). Left panel, control mice; Middle panel, note the extreme impact of DSS-induced loss of barrier and inflammation on colon integrity; red arrows illustrate the loss of the distal colon in DSS-treated animals, but not in DSS-treated animals if this circuit was activated (right panel, n=4; similar protection is observed in all animals). Panel e, Bar graphs show levels of CXCL-1 pro-inflammatory cytokine in control, in the DSS-model and in the DSS-model in combination with activation of TRPA1 vagal neurons. Values are means±SEM; Mann-Whitney U-test, p=0.03. Panel f, Significant levels of occult stool blood is detected in the DSS-model but not in the TRPA1 neuron-activated animals. Values are means±SEM; Mann-Whitney U-test, p=0.03.

FIG. 32 shows cNST neurons activated in response to LPS. Panel a, Schematic of Fos induction by LPS stimulation. Mice received an intraperitoneal injection with LPS, and two hours later, brains were extracted, sliced and immune-stained for Fos expression. Panel b, Shown is Fos expression in six 100 μm coronal sections, each 300 μm apart from bregma −8.1 mm to bregma −6.8 mm. Note the selective induction of Fos in the cNST but not in the rostral nucleus of the solitary tract (rNST). Scale bars, 200 μm. Similar results were observed in multiple animals (n=4).

FIG. 33 shows normal Fos induction to LPS is lacking in the cNST of Myd88 knockouts. Panel a, Blocking LPS signaling abrogates Fos induction in response to LPS. WT and Myd88−/− mice were injected with LPS intraperitoneally, and two hours later, brains were extracted, sliced and stained for Fos expression (See FIG. 26 Panel b). As a control, WT mice were injected with saline. Bilateral Fos expression is strongly induced by LPS in the cNST of WT mice but largely absent from Myd88−/− mice; n=4 mice each. The right panel shows the quantification of Fos-positive neurons. The equivalent area of the cNST (200 μm×200 μm, bregma −7.5 mm) was processed, and positive neurons were counted. Values are means±SEM; ANOVA with Tukey's honestly significant difference (HSD) post hoc test, p<0.0001 (Saline vs LPS); p<0.0001 (LPS vs Myd88−/−+LPS). Scale bar, 200 μm. Panel b, Myd88 knockouts have impaired cytokine responses to LPS. WT and Myd88−/− mice received an intraperitoneal injection of LPS, and peripheral blood was taken 2 h later to measure circulating levels of pro-inflammatory (IL-6, IL-1β, TNF-α) and anti-inflammatory (IL-10) cytokines by ELISA. As a control, WT mice were injected with saline. Note that cytokine induction is dramatically reduced in Myd88−/− mice. n=4 mice each group. Values are means±SEM; ANOVA with Tukey's HSD post hoc test, LPS vs Myd88−/−+LPS: p<0.0001 (IL-6); p<0.01 (IL-1β); p<0.01 (TNF-α); p<0.01 (IL-10). No significant difference was observed between Saline and Myd88−/−+LPS: p=0.19 (IL-6), p=0.88 (IL-1β), p=0.52 (TNF-α); p=0.96 (IL-10).

FIG. 34 shows Fos induction of cNST neurons by a variety of immune insults. Panel a, Schematic of Fos induction by immune stimulation. Mice received an intraperitoneal injection with a variety of different immune insults, and two hours later, brains were extracted, sliced and immune-stained for Fos expression. Panel b, Shown are examples for LPS (50 μg kg-1), Lipoteichoic acid (LTA, 1 mg kg-1), Flagellin (20 μg kg-1), Profilin (20 μg kg-1) and Zymosan (2.5 mg kg-1). All robustly activated Fos in the cNST (outlined in yellow). Scale bar, 200 μm. Panel c, Schematic illustrating experimental procedures to TRAP cNST neurons activated by LPS. The LPS-induced TRAPed neurons were genetically labeled with a Cre-dependent fluorescent reporter (tdTomato, Ai941). TRAP2; Ai9 mice were stimulated intraperitoneally with LPS (50 μg kg-1) or control (saline) stimulus, followed by injection of 4-OHT 90 mins later. After 7 days, the brains were sectioned and examined for the induction of the tdTomato reporter. Panel d, Shown are coronal sections of cNST after TRAP2; Ai9 animals were TRAPed with LPS or Saline. Each panel is a confocal maximal projection image from Bregma −7.5 mm. Shown are data representing 3 different animals, in independent experiments. Note that LPS but not saline led to consistent and robust bilateral TRAP labelling of neurons in the cNST (outlined in yellow) across animals. Scale bars, 200 μm.

FIG. 35 shows activation of LPS-TRAPed neurons in the cNST does not elicit immune responses in the absence of immune challenge. Panel a, Schematic of chemogenetic activation strategy. AAV viruses carrying a control mCherry construct, or the hM3Dq excitatory DREADD, were targeted to the cNST of TRAP2 mice for chemogenetic activation. Mice were TRAPed with LPS (50 μg kg-1). After 4 weeks, cytokine responses to saline (e.g., without LPS) was quantified in the presence of DREADD agonist, CNO. Panel b, Shown are levels of anti-inflammatory (IL-10) and pro-inflammatory (IL-6, IL-1β) cytokines in the peripheral blood of mice expressing excitatory DREADD (hM3Dq), or control (mCherry), in the LPS-TRAPed cNST neurons. All mice were injected with CNO 1 h prior to saline stimulation. The data presented comes from FIG. 27 and replotted with an expanded y axis. Note that in the absence of the immune stimuli (LPS), activation of this circuit produces no meaningful effect on circulating cytokine levels. Grey bars (control), TRAP2 animals injected with DIO-mCherry (n=6); black bars (hM3Dq), TRAP2 animals injected with DIO-hM3Dq (n=6). Values are means±SEM; Mann-Whitney U-tests, p=0.17 (IL-6), p=0.93 (IL-1β), p=0.93 (TNF-α), p=0.37 (IL-10).

FIG. 36 shows LPS-TRAPed neurons in a GABAergic neuronal cluster in the cNST. Panel a, Single-cell RNA sequencing (scRNA-seq) cataloging neuronal clusters in the cNST. A uniform manifold approximation and projection (UMAP) plot of transcriptomic data revealed 14 Glutamatergic neuronal clusters (1-14, colored) and 6 GABAergic clusters (15-20, grey). Panel b, ScRNA-seq of individual LPS-TRAPed neurons from the cNST. The tdTomato-labeled LPS-TRAPed cells were isolated by FACS and individually sequenced. The UMAP of LPS-TRAPed neurons was then superimposed onto the UMAP of the entire cNST map (FIG. 36A), showing that in addition to excitatory clusters (7, 10, 12, red; see FIG. 28B.), an inhibitory cluster (15, black) also contains LPS-TRAPed neurons. Activation of this inhibitory cluster has no effect on cytokine levels after LPS injection (see FIG. 37).

FIG. 37 shows activation of excitatory but not inhibitory neurons suppresses LPS-induced inflammation. Panel a, Activation of cNST glutamatergic neurons suppresses LPS-induced inflammation. Upper panels, UMAP plot of the normalized expression of Slc17a6 (also known as Vglut2) highlighting glutamatergic (excitatory) neuronal clusters in the cNST; also illustrated is the strategy for chemogenetic activation of the excitatory cNST neurons. An AAV virus carrying the excitatory Cre-dependent hM3Dq DREADD (AAV-DIO-hM3Dq) was targeted bilaterally to the cNST of Vglut2-cre mice. lower panels, shown are circulating levels of anti-inflammatory (IL-10) and pro-inflammatory (IL-6, IL-1β) cytokines in the peripheral blood of LPS-stimulated mice expressing excitatory DREADD (hM3Dq), or control (mCherry), in glutamatergic neurons. All animals were given CNO 1 h prior to the LPS injection. n=7 animals for each group. Values are means±SEM; Mann-Whitney U-tests, p=0.002 (IL-6), p=0.004 (IL-1β), p=0.02 (IL-10). Note the increase in the levels of anti-inflammatory (compare grey and green bars), and decrease in the levels of pro-inflammatory cytokines (compare gray and red bars). Panel b, Upper panels, UMAP plot of the normalized expression of Slc32a1 (also known as Vgat) highlighting the GABAergic (inhibitory) neuronal clusters in the cNST, and the chemogenetic strategy for activation of the inhibitory cNST neurons. Lower panels, shown are levels of anti-inflammatory (IL-10) and pro-inflammatory (IL-6, IL-1β) cytokines in the peripheral blood of mice after LPS-stimulation, both in mice expressing excitatory DREADD (hM3Dq), or control (mCherry) in GABAergic neurons. n=4 mice for hM3Dq group and 5 mice for control (mCherry) group. Values are means±SEM; Mann-Whitney U-tests, p=0.73 (IL-6), p=0.90 (IL-1β), p=0.73 (IL-10). Note that activation of cNST GABAergic neurons does not meaningfully impact LPS-induced inflammation.

FIG. 38 shows DBH is selectively expressed in the cNST. Panel a, Diagram of a coronal section highlighting the cNST (in yellow) and the AP (in blue). Panels b-d, First, DBH expression was examined by using DBH-cre mice, crossed to the Ai9 tdT-reporter line. Most labeling was detected in the cNST (n=4 mice), with very minimal expression in the AP in the adult brain, and some of this may reflect limited expression during development (i.e., the cre reporter acting as a lineage tracer). Next, a cre-dependent mCherry-reporter virus (AAV9-Syn-DIO-mCherry) was directly injected into the cNST and AP of adult mice, and nearly all of the labeling is detected in the cNST, with almost no expression in the AP (n=4 mice). Finally, in-situ hybridizations were performed, and expression is largely restricted to the cNST, with very low levels in the AP. Scale bars, 200 μm. Shown in the bar graphs are the quantitation of DBH expressing neurons in cNST vs AP. Panel e, Sample brain demonstrating expression of GCaMP6s restricted to the cNST, with minimal expression in the AP; the image also demarks the location of the recording fiber (dashed rectangle). Scale bar, 100 μm. Similar results were observed in the analyzed animals, both control and vagotomized (n=6 each).

FIG. 39 shows immune insults activate DBH neurons in the cNST. Panel a, Schematic illustrating Fos induction in DBH neurons in the cNST by LPS and cytokine stimulation. Dbh-cre mice were injected intraperitoneally with LPS, IL-10 or a cocktail of IL-6, IL-1β, and TNF-α, and brain slices were analyzed for Fos and Dbh labeling. DBH neurons were marked by tdTomato (tdT) expression (Ai9 reporter line40), and Fos by immunohistochemistry. Panels b-c, Coronal sections of the brain stem showing neurons expressing DBH (Dbh-tdT, red) and neurons activated by LPS, IL-10 (anti-inflammatory, middle row), or by a cocktail of 3 pro-inflammatory cytokines (100 μg kg-1, TNF-α, 100 μg kg-1, IL-6, 100 μg kg-1, IL-1β, bottom row). Note that all three stimuli activate DBH neurons. Scale bar, 200 μm. Panel d, Quantification of the fraction of DBH neurons that express immune-induced Fos (n=4 mice each group). Anti=IL10, pro=a mixture of TNF-α, IL-6, and IL-1β. Values are means±SEM; ANOVA with Tukey's HSD post hoc test, LPS vs Saline: p<0.0001; Anti vs Saline: p<0.0001; Pro vs Saline: p<0.0001.

FIG. 40 shows ablation of DBH cNST neurons increases inflammatory responses. Panel a, Anti-DBH Saporin (SAP) was injected bilaterally into the cNST to selectively ablate DBH neurons; control mice were injected with PBS. The bar graphs show circulating levels of pro-inflammatory (IL-6, TNF-α) and anti-inflammatory (IL-10) cytokines in the peripheral blood of control mice, and DBH-ablated animals (Dbh-SAP) after LPS stimulation. Note the significant increase in the levels of IL-6 and TNF-α after LPS stimulation in Anti-DBH Saporin mice versus control animals (n=5 each group). As seen with the TRAPped cNST neurons (FIG. 27B), the level of IL-10 is greatly reduced in the ablated mice (n=5 each group). Values are means±SEM; Mann-Whitney U-tests, IL-6: p=0.02; TNF-α: p=0.04; IL-10: p=0.02. Panel b, Loss of DBH neurons in the cNST after Dbh-SAP induced cell-death. Upper panel, diagram of a coronal section highlighting the cNST (in yellow). Lower panels show in situ hybridization signals for Dbh RNA in the cNST of control and Dbh-SAP treated mice. Note the dramatic loss of DBH neurons in the cNST of the experimental animals (compare right panel with left control); similar results were observed in independently injected animals; the bar graph shows quantitation for 5 animals. PBS, control animals injected with PBS; DBH-Ablation, animals injected with Dbh-SAP (see Methods for details). Scale bars: 200 μm.

FIG. 41 shows vagal responses to anti-inflammatory and pro-inflammatory cytokines. Panel a, Schematic of vagal calcium imaging while simultaneously delivering cytokines onto the intestines. Panel b, The micrograph shows a representative view of a nodose ganglion from Vglut2-cre; Ai96 during an imaging session. All vagal sensory neurons express GCaMP6s. Right panels show representative traces from vagal neurons selectively responding to anti-inflammatory (IL-10, upper panels) and pro-inflammatory (IL6, lower panels) cytokines. Each cytokine was perfused for 180 seconds (starting at the time indicated by the color arrows; green, IL-10; red, IL-6) in 2 repeat trials. Scale bar, 100 μm. Summary data is presented in FIG. 29B. c, Responses of TRPA1 vagal neurons to anti-inflammatory cytokines. The micrograph depicts a sample nodose ganglion from Trpa1-cre; Ai162 during an imaging session. Right panels are sample traces from two neurons. Scale bar, 100 μm.

FIG. 42 shows vagal responses to IL-6, TNF-α, IL-1β. Schematic of vagal calcium imaging while simultaneously delivering cytokines onto the intestines. (see FIG. 29). The heat maps depict z-score-normalized fluorescence traces from vagal neurons responding to individual pro-inflammatory cytokines. Each row represents the averaged activity of a single cell to 2 trials. Dashed lines denote stimulus time window (180 sec). n=5 mice.

FIG. 43 shows neuronal clusters in the Vagal ganglia. Panel a, Strategy for chemogenetic activation of vagal neuronal populations. An excitatory DREADD receptor (via AAV-DIO-hM3Dq) was targeted bilaterally to the nodose ganglia of Vip-cre, Gpr65-cre, Piezo2-cre and Oxtr-cre mice. The mice were then examined for changes in circulating cytokine levels in response to LPS in the presence of the DREADD receptor agonist CNO. Panels b-e, The bar graphs show cytokine levels of IL-6, IL-1β and IL-10 in the peripheral blood of mice expressing either excitatory DREADD (hM3Dq) or control mCherry in VIP, GPR65, PIEZO2, OXTR vagal neurons, 2 hours after LPS stimulation. All mice were injected with CNO 1 hour prior to LPS. Panel b, Vip: n=4 each group; Mann-Whitney U-tests, p (IL-6)=0.88, p (IL-1β)=0.88, p (IL-10)=0.2. Panel c, Gpr65: n=5 (control) and 4 (hM3Dq); Mann-Whitney U-tests, p (IL-6)=0.03, p (IL-1β)=0.06, p (IL-10)=0.55. Panel d, Piezo2: n=5 each group; Mann-Whitney U-tests, p (IL-6)=0.54, p (IL-1β)=0.42, p (IL-10)=0.42. Panel e, Oxtr: n=5 each group; Mann-Whitney U-tests, p (IL-6)=0.84, p (IL-1β)=0.65, p (IL-10)=0.84. Values are means±SEM; Activation of any of these vagal populations has no appreciable effect on LPS-induced cytokine responses.

FIG. 44 shows enhancement of the anti-inflammatory response does not rely on the reduction of pro-inflammatory cytokines. Panel a, AAV viruses carrying a control mCherry construct, or the hM3Dq excitatory DREADD, were targeted bilaterally to the nodose ganglion of Trpa1-cre mice for chemogenetic activation. All of the mice received an intraperitoneal injection of LPS to elicit an inflammatory response. Animals were then divided into 2 groups: control (no clamping), and the experimental (Pro-inflammatory clamping) where they were additionally injected with high levels of a pro-inflammatory cytokine cocktail (IL-6, IL-1β, TNF-α) to “clamp”, and thus maintain a high pro-inflammatory state (see Methods). Animals with activated TRPA1 vagal neurons in the control group (e.g., only injected with LPS) exhibited the expected enhancement in circulating IL-10 and a reduction in the levels of the pro-inflammatory cytokines. Notably, the levels of IL-10 remain similarly enhanced by TRPA1 vagal stimulation, even when the levels of pro-inflammatory cytokines are not suppressed. Blood samples were collected 2 hours after LPS stimulation, and all animals were given CNO 1 hr prior to LPS injection. n=5 mice (mCherry No “Clamping”); n=4 for all other groups. Values are means±SEM; Mann-Whitney U-tests, IL-10 levels in mCherry No “Clamping” vs mCherry Pro-inflammatory “Clamping”, p=0.99; IL-10 levels in hM3Dq No “Clamping” vs hM3Dq Pro-inflammatory “Clamping”, p=0.20. Panel b, Levels of circulating IL-6, TNF-α, IL-10 in mice following an intraperitoneal injection of exogenous IL-6 (100 μg kg-1), TNF-α (100 μg kg-1), or IL-10 (100 μg kg-1), taken 10 mins or 2 hrs after the injection (n=5 mice each group). Also shown are the levels of the same cytokines after LPS (2 hrs). Values are means±SEM.

FIG. 45 shows IL-10 and fat activate distinct subsets of TRPA1 vagal neurons. The heat map shows that the TRPA1 neurons that selectively responded to extraintestinal application of IL-10 (top panel), are separate from the pool of neurons that responded to fat (LA, middle panel). Shown are the responses of 63 TRPA1-labeled vagal neurons to anti-inflammatory stimuli and to intestinal delivery of fat. Heat maps depict z-score-normalized responses to stimuli of IL-10 (1 μg ml-1) and fat (LA, 10% linoleic acid). IL-10 was perfused onto the intestines for 180 s (dashed lines) and linoleic acid was infused into the gut for 10 s (dashed lines). Each row represents the average activity of a different neuron during two exposures to the stimulus. n=4 mice. Shown also are 2 neurons that appeared to respond to both stimuli (bottom panel); given that these represent less than 1 neuron per animal they were not considered further.

FIG. 46 shows ablation of TRPA1 vagal neurons prevents the emergence of a normal anti-inflammatory response. Panel a, Ablation of TRPA1 vagal neurons block the induction of Fos in response to IL-10 stimulation. Diphtheria toxin (DTX) was injected bilaterally into the nodose ganglion of Trpa1-cre; Rosa-DTR mice76 to selectively ablate TRPA1 vagal neurons. Control animals received injection of PBS. Mice were then examined for cNST Fos induction 2 hours following intraperitoneal injection of IL-10 (see FIG. 39). IL-10 stimulation induces significant Fos labelling in control but not in animals lacking vagal TRPA1 neurons (Trpa1-Ablated). The right panel shows the quantification of Fos-positive neurons (n=4 mice each). The equivalent area of the cNST (200 μm×200 μm, bregma −7.5 mm) was processed. Values are means±SEM; Mann-Whitney U-test, p=0.03. Panel b, Ablation of TRPA1 vagal neurons prevents a normal anti-inflammatory response. Bar graphs show the levels of anti-inflammatory (IL-10) and pro-inflammatory (IL-6, IL-1/p) cytokines in the peripheral blood of control mice, and TRPA1-ablated animals (Trpa1-ablated) after LPS stimulation. Note the significant change in the levels of IL-10 after LPS stimulation in mice missing TRPA1 vagal neurons versus control animals (n=4). By contrast, the levels of IL-6 and IL-1β are largely unaffected in mice lacking TRPA1 vagal neurons. Values are means±SEM; Mann-Whitney U-tests, IL-10: p=0.03; IL-6: p=0.90; IL-1β: p=0.90.

FIG. 47 shows a vagal to cNST circuit. Panel a, Strategy for targeting a green fluorescently labelled retrograde transsynaptic rabies reporter (RABV-GFP) to the cNST. DBH neurons in the cNST (in DBH-cre animals) were infected with AAV-Flex-G-mKate (red fluorescence) and AAV-Flex-TVA-mCherry (also red fluorescence) viruses. The targeted expression of the G-protein and the TVA receptor allows monosynaptic transfer and expression of the RABV-GFP retrograde virus. Panel b, DBH Neurons in the cNST that were co-infected by the AAV-G and AAV-TVA viruses and by the RABV-GFP retrograde reporter are highlighted with asterisks. Panel c, DBH neurons receive monosynaptic input from CALCA vagal neurons. RNA fluorescence in situ hybridization (in situs) marking CALCA neurons (left panel, red) and GFP from the retrograde virus (right panel, green), demonstrating that CALCA neurons in the nodose ganglion directly project to DBH neurons in the cNST. The CALCA vagal neurons co-labeled with RABV-GFP are indicated by asterisks. n=3 mice, Scale bars, 50 μm. Panel d, DBH neurons receive monosynaptic input from TRPA1 vagal neurons. RABV-GFP from DBH neurons in the cNST retrogradely labels TRPA1 vagal neurons. The TRPA1 vagal neurons co-labeled with RABV-GFP are indicated by asterisks. n=3 mice, Scale bars, 50 μm. Note that about 10-20% of the TRPA1 neurons are labeled, and there is a significant number of neurons labeled by the retrograde virus but not expressing TRPA1 (same observation for CALCA neurons). This is expected due to multiple reasons: (i) the limited efficiency of the TVA retrograde labeling system, (ii) TRPA1 neurons represent multiple functional types, for example those that carry signals informing the brain of intestinal fat versus those that report inflammatory responses (see FIG. 45), and (iii) DBH neurons would be expected to get inputs from other vagal neurons (for instance CALCA and others). Panel e, Stimulation of TRPA1 vagal neurons activates DBH neurons in the cNST. An excitatory DREADD receptor (via AAV-DIO-hM3Dq) was targeted bilaterally to the nodose ganglion of Trpa1-cre mice. The mice were then examined for the induction of Fos in the cNST. Lower panels show in-situ hybridizations for Dbh (red) and Fos (green). Scale bars, 50 μm. Approximately 40% of DBH neurons are activated in response to TRPA1 neuron stimulation (bar graph).

FIG. 48 shows activation of TRPA1 vagal neurons impacts the course of Salmonella infection. Panel a, Strategy for chemogenetic activation of TRPA1 vagal neurons. An excitatory DREADD receptor (via AAV-DIO-hM3Dq) was targeted bilaterally to the nodose ganglion of Trpa1-cre mice. Control mice received injections of AAV-DIO-mCherry. Mice were infected with STm (1˜2×10\hat{ }7 CFU) via oral gavage. The circuit was maximally activated by injecting CNO at 12 h intervals for a total of 8 injection beginning 12 h prior to STm infection. Body weight was monitored daily. Panel b, Activation of TRPA1 vagal neurons impairs protection against STm infection. Left graph shows the load of STm in the spleens and mesenteric lymph nodes (LNs) of mice expressing the excitatory DREADD (hM3Dq; n=4 mice) or control reporter (mCherry; n=4 mice). Note the nearly 2-log increase in STm load in the spleen and LN of TRPA1-activated animals, reflecting the suppressed pro-inflammatory state. Values are means±SEM; Mann-Whitney U-test, spleen, p=0.03; LN, p=0.03. Panel c, These animals also experienced a severe loss of body weight during the course of STm infection (right panel).

FIG. 49 shows activation of the vagal-brain axis does not change the levels of circulating corticosterone induced by LPS. Panel a, An AAV virus carrying the hM3Dq excitatory DREADD, was targeted to the cNST of Dbh-cre mice for chemogenetic activation. Control animals received an injection of AAV-DIO-mCherry. The bar graphs show levels of corticosterone in the peripheral blood of mice expressing the excitatory DREADD (hM3Dq; n=4 mice) or control reporter (mCherry; n=4 mice). Blood samples were collected 2 hours after LPS stimulation, and all animals were given CNO 1 hr prior to LPS injection. Values are means±SEM; Mann-Whitney U-test, p=0.32. Panel b, An AAV virus carrying the hM3Dq excitatory DREADD, was targeted to the TRPA1 vagal neurons for chemogenetic activation. The bar graphs show the levels of corticosterone in the peripheral blood of the mice expressing the excitatory DREADD (hM3Dq; n=5 mice) or control reporter (mCherry; n=5 mice). Values are means±SEM; Mann-Whitney U-tests, p=0.84.

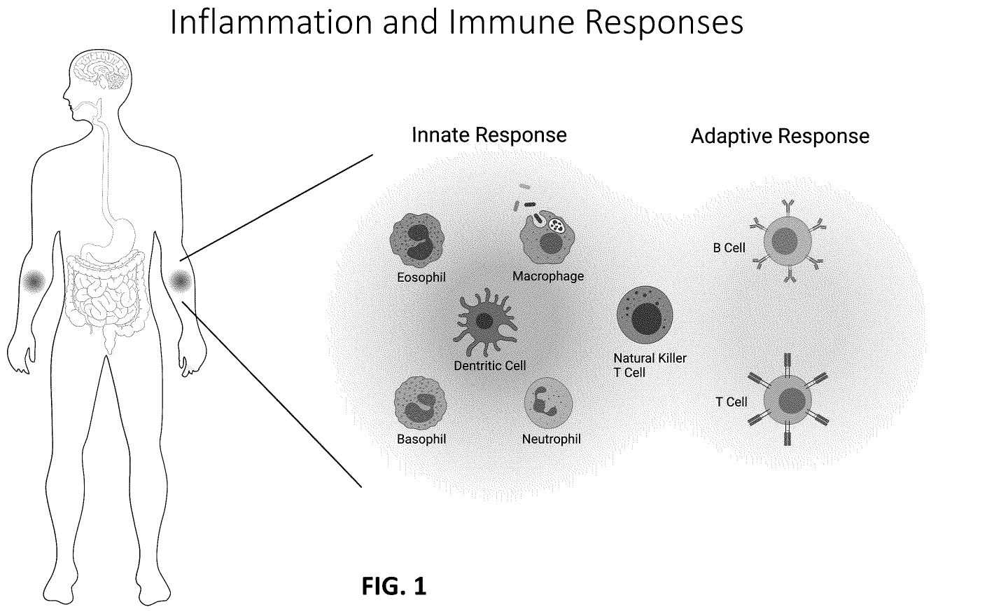

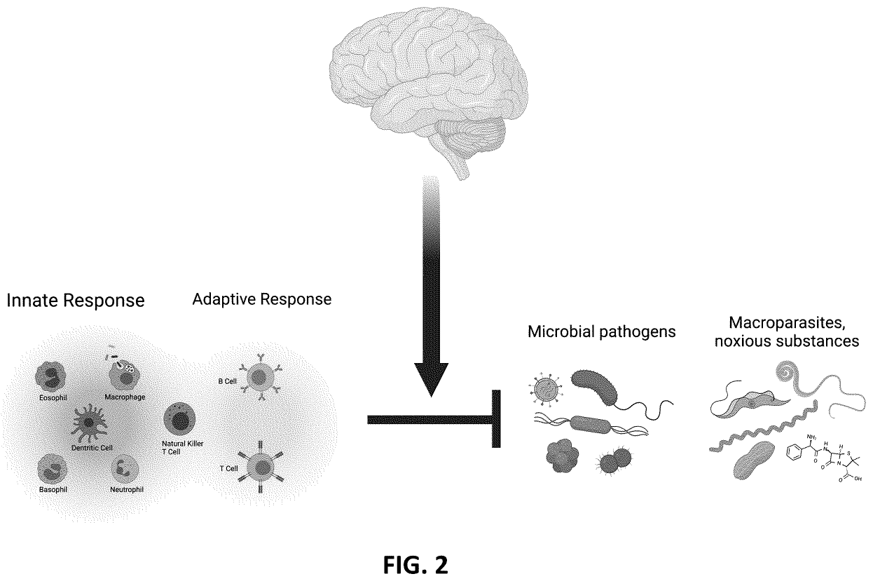

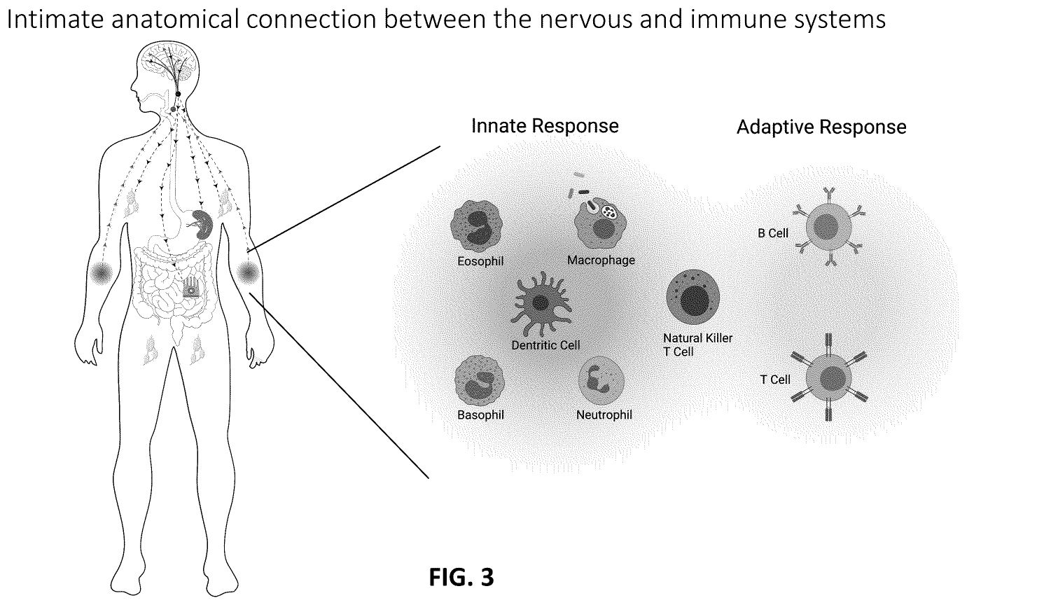

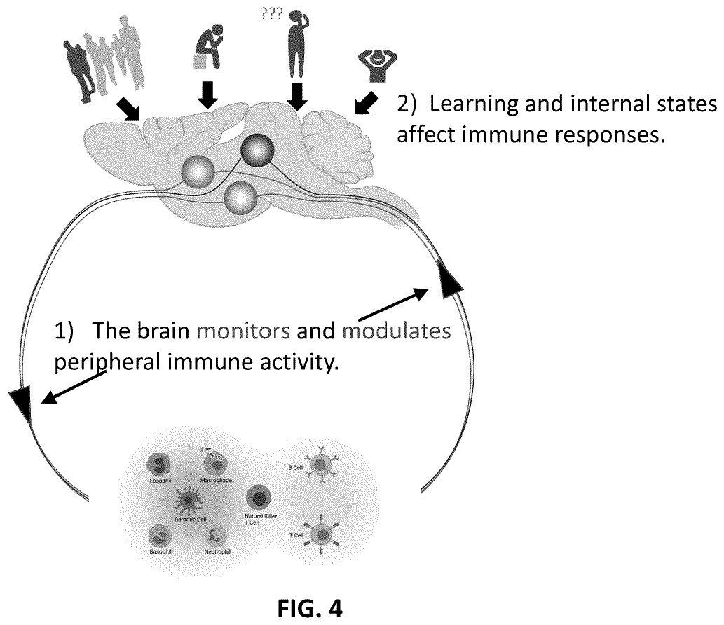

### DETAILED DESCRIPTION OF ILLUSTRATIVE EMBODIMENTS

The disclosed methods may be understood more readily by reference to the following detailed description taken in connection with the accompanying figures and examples, which form a part of this disclosure. It is to be understood that these methods are not limited to the specific methods described and/or shown herein, and that the terminology used herein is for the purpose of describing particular embodiments by way of example only and is not intended to be limiting of the claimed methods. Also, as used in the specification including the appended claims, the singular forms “a,” “an,” and “the” include the plural, and reference to a particular numerical value includes at least that particular value, unless the context clearly dictates otherwise. The term “plurality,” as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the methods which are, for clarity, described herein in the context of separate embodiments, may also be provided in combination in a single embodiment. Conversely, various features of the methods that are, for brevity, described in the context of a single embodiment, may also be provided separately or in any subcombination. Further, reference to values stated in ranges include each and every value within that range.

Described herein are methods of regulating an immune response. In some embodiments, methods of regulating an immune response in a mammal comprise modulating the activity of one or more glutamatergic neurons. In some embodiments, methods of regulating an immune response in a mammal comprise modulating the activity of one or more dopamine beta-hydroxylase (Dbh)-positive neurons. In some embodiments, the modulated neurons are present in the caudal nucleus of the solitary tract (cNST) of a subject. In some embodiments, activation of the glutamatergic neurons suppresses an immune response. In some embodiments, activation of the neurons results in a decreased serum level of one or more proinflammatory cytokines and/or an increased serum level of one or more anti-inflammatory cytokines.

In some embodiments, the methods of regulating an immune response in a mammal comprise modulating the activity of one or more TRPA1-expressing neurons in a vagal ganglion of the mammal to thereby regulate the immune response, wherein, when the neuron is engineered to express a designer receptor exclusively activated by a designer drug (DREADD), there is a decreased serum level of one or more proinflammatory cytokines and/or an increased serum level of one or more anti-inflammatory cytokines in the presence of a DREADD ligand.

In some embodiments, the methods of regulating an immune response in a mammal comprise modulating the activity of one or more CGRP receptor-expressing neurons in a vagal ganglion of the mammal to thereby regulate the immune response, wherein, when the neuron is engineered to express a DREADD, there is an increased serum level of one or more proinflammatory cytokines and/or a decreased serum level of one or more anti-inflammatory cytokines in the presence of a DREADD ligand.

In some embodiments, the methods of regulating an immune response in a mammal comprise modulating the activity of one or more neurons in the brainstem of the mammal, wherein, the neuron is activated by lipopolysaccharide (LPS) to induce release of pro-inflammatory cytokines, and wherein, when the neuron is engineered to express a DREADD, there is a reduced LPS-evoked release of pro-inflammatory cytokines and/or increased release of anti-inflammatory cytokines in the presence of a DREADD ligand.

Modulation of the neurons engineered to express a DREADD herein can result in an increased or decreased serum level of one or more proinflammatory cytokines and/or an increased or decreased serum level of one or more anti-inflammatory cytokines. The pro-inflammatory cytokines can comprise one or more of tumor necrosis factor, interleukin-6, and interleukin-1. The anti-inflammatory cytokines can comprise one or more of interleukin-10, interleukin-4, and tumor growth factor beta.

According to any one of the herein described methods, modulating the activity of one or more neurons can comprise activating the neuron. According to any one of the herein described methods, modulating the activity of one or more neurons can comprise inhibiting the neuron. Modulating the activity of one or more neurons according to any of the herein described methods can comprise administering an agent to the mammal. The agent modulating the activity of one or more neurons can be a small molecule. The agent modulating the activity of one or more neurons can be a biologic. In some embodiments, the agent activates the neuron. In some embodiments, the agent inhibits the neuron.

According to any one of the herein described methods, regulating the immune response comprises increasing or reducing a serum level of one or more pro-inflammatory cytokines and/or increasing or reducing a serum level of one or more anti-inflammatory cytokines. In some embodiments, the serum level of one or more pro-inflammatory cytokines is increased. In some embodiments, the serum level of one or more pro-inflammatory cytokines is decreased. In some embodiments, the serum level of one or more anti-inflammatory cytokines is increased. In some embodiments, the serum level of one or more anti-inflammatory cytokines is decreased. Changes in the levels of pro-inflammatory cytokines can accompany a change in the levels of anti-inflammatory cytokines. For example, decreased levels of one or more pro-inflammatory cytokines can accompany increased levels of anti-inflammatory cytokines or increased levels of one or more pro-inflammatory cytokines can accompany decreased levels of anti-inflammatory cytokines. The pro-inflammatory cytokines can comprise one or more of tumor necrosis factor, interleukin-6, and interleukin-1. The anti-inflammatory cytokines can comprise one or more of interleukin-10, interleukin-4, and tumor growth factor beta.

The DREADD used in any one of the herein described methods can be hM3Dq, hM1Dq, or hMD5q. In some embodiments, the DREADD is hM3Dq. In some embodiments, the DREADD is hM1Dq. In some embodiments, the DREADD is hMD5q. The DREADD ligand can be clozapine N-oxide (CNO).

According to the herein described methods, the mammal can have a cancerous disease, an autoimmune disease, an infectious disease, or an injury. In some embodiments, the mammal has a cancerous disease. In some embodiments, the mammal has an autoimmune disease. In some embodiments, the mammal has an autoimmune disease. In some embodiments, the mammal has an infectious disease. In some embodiments, the mammal has an injury.

Also disclosed herein are methods to alter an immune response via chemogenetic activation of neurons in the mammal's caudal nucleus of the solitary tract. Regulation of the immune system encompasses means by which the immune response can be altered from its otherwise natural state. In some embodiments, alteration can include inducing, enhancing, or sustaining an immune response. In some embodiments, alteration can include preventing, suppressing, or shortening an immune response.

Also disclosed herein are methods to treat or suppress an inflammatory response in a mammal via chemogenetic activation of neurons in the mammal's caudal nucleus of the solitary tract. Further disclosed herein are methods to treat or suppress an inflammatory response in a mammal by other methods of activation of the relevant neurons, such as with agents (e.g., small molecules, biologics such as antibodies, or other agents). Treatment as used herein describes the amelioration of a sign, symptom, or underling cause of a disease or condition. An inflammatory response can encompass any sign, symptom, or underlying cause related to immune system activation.

Chemogenetic activation in this method can be accomplished through a DREADD. Examples of DREADDs include, but are not limited to hM3Dq, hM1Dq, or hMD5q. Transformation of nucleic acids encoding a DREADD into neurons of interest can be accomplished though any means sufficient for transfection of nucleic acid into cells. Chemogenetic activation via a DREADD employs a ligand to which the DREADD can respond by activation. Exemplary ligands include, but are not limited to, clozapine N-oxide.

Activation of neurons (e.g., via chemogenetic activation, immunostimulation, or by another method such as administration of an agent, electrical stimulation, magnetic stimulation, or mechanical stimulation) can be verified or measured by any established method for determining or imaging neuronal activity including but not limited to assessment of c-fos induction or targeted recombination in active populations (TRAP). Activation of neurons can also be verified by measuring or detecting calcium response (e.g., using a calcium indicator such as a genetically encoded fluorescent or luminescent calcium indicator) in the relevant neurons.

The disclosed methods can result in or be used to reduce the levels of pro-inflammatory cytokines that exist in the serum or blood of a mammal. Pro-inflammatory cytokines are those capable of inducing, enhancing, or sustaining an immune response. Examples of pro-inflammatory cytokines include, but are not limited to, tumor necrosis factor, interleukin-6, and interleukin-1.

The disclosed methods can result in or be used to increase the levels of anti-inflammatory cytokines that exist in the serum or blood of a mammal. Anti-inflammatory cytokines are those capable of preventing, suppressing, or shortening an immune response. Examples of anti-inflammatory cytokines include, but are not limited interleukin-10, interleukin-4, and tumor growth factor beta.

The disclosed methods can be used on a mammal that has a disease, injury, or other state where immune regulation serves a purpose. Due to the role of the immune system in disease and injury states, altering an immune response in a manner beneficial to amelioration of a disease or injury state is contemplated by the disclosure. Regulation of the immune response can be for purposes that include but are not limited to inducing or preventing an immune response, activating or suppressing and immune response, or sustaining or shortening an immune response. Disease states that could benefit from regulation of an immune response include, but are not limited to cancerous diseases, autoimmune diseases, or infectious diseases. Any disease, or symptom thereof, which can be improved by immune system regulation or treatment and suppression of an inflammatory response could benefit from the described methods.

Animals suitable for the methods described include mammals, especially those with brain regions that contain a caudal nucleus of the solitary tract and vagus nerve or homologous structures whose activation can regulate the immune response.

Also disclosed herein are model animals which can be useful in the study of the regulation of an immune response as described herein, or in methods of screening therapeutic compounds and other stimuli (e.g., electrical, magnetic, or mechanical stimuli) for their ability to regulate an immune response. In some embodiments, a model animal comprises an indicator capable of emitting or producing a detectable signal when neurons of interest are activated. In some embodiments, the model animal comprises a genetically encoded calcium indicator (GECI) in the neurons of interest. In some embodiments, the GECI is a fluorescent or luminescent calcium indicator. In some embodiments, the GECI is a GCaMP. In some embodiments, the neurons of interest are glutamatergic neurons. In some embodiments, the neurons of interest are those which express vlgut2 (i.e., vglut2-positive neurons). In some embodiments, the neurons of interest are those which express Dbh (i.e., Dbh-positive neurons). In some embodiments, the neurons of interest are present in the caudal nucleus of the solitary tract (cNST) of the animal. In some embodiments, the animal is one having a caudal nucleus of the solitary tract (cNST). In some embodiments, the animal is a mammal. In some embodiments, the animal is a mouse.

In some embodiments, the indicator (e.g., the GECI) is expressed only or substantially only in the neurons of interest. For example, the GECI may be expressed at a level in the neurons of interest (e.g., Dbh-positive neurons) which is at least 10-fold, 20-fold, 30-fold, 40-fold, 50-fold, 100-fold, or 1000-fold more than in other neurons. In some embodiments, the indicator (e.g., the GECI) is targeted to the neurons of interest (e.g., the Dbh-positive neurons). In some embodiments, the indicator (e.g., the GECI) is preferentially expressed in the neurons of interest (e.g., the Dbh-positive neurons). In some embodiments, a different indicator (e.g., a different GECI) can be expressed in non-target neurons. In such embodiments, this can allow for ready determination of activation of both target and non-target neurons.

Model animals according to the instant disclosure can be prepared by a variety of methods, including those described herein. For example, in some embodiments, a model animal is prepared from an animal engineered to express a recombinase in an endogenous locus of a biomarker associated with a neuron of interest (e.g., as in vglut2-cre and Dbh-cre mice described herein). Targeted expression of the GECI in the neurons of interest can then be accomplished by introducing a vector (e.g., an AAV vector) to the relevant cells which expresses the GECI under control of the recombinase. Other methods of generating such model animals are also contemplated as within the scope of the instant disclosure.

Also disclosed herein are methods of screening therapeutic compounds (and other stimuli, such as electrical, magnetic, or mechanical stimulation) to assess their ability to alter the immune response. In some embodiments, the screening method comprises detecting signal in neuronal subsets activated by pro-inflammatory cytokines and neuronal subsets activated by anti-inflammatory cytokines as described herein following administration of a therapeutic agent.

In one aspect herein is a method of assessing the ability of a stimulus to regulate an immune response. In some embodiments, the method comprises applying the stimulus to a subject. In some embodiments, the method comprises measuring in the subject, in response to the stimulus, activation of neurons associated with modulation of the immune response (e.g., any of the neurons described herein, such as glutamatergic neurons (e.g., those of the cNST), including Dbh-positive neurons and/or vglut2-positive neurons). In some embodiments, the method comprises measuring activation of glutamatergic neurons In some embodiments, the method comprises measuring activation of Dbh-positive neurons. In some embodiments, the method comprises measuring activation of vglut2-positive neurons. In some embodiments, the stimulus can be identified as capable of regulating the immune response based on the activation of the relevant neurons.

In some embodiments, the method comprises measuring in the subject the effect of the stimulus on non-target neurons. For example, in methods in which it is desired to assess the activation of Dbh-positive neurons, the method can also comprise measuring the effect of the stimulus on Dbh-negative neurons (e.g., those expressing no Dbh or those expressing low levels of Dbh (e.g., non-readily detectable levels of Dbh). In such embodiments, the method can comprise comparing the activation by the stimulus the target neurons and non-target neurons (e.g., the method comprises comparing activation by the stimulus of the Dbh-positive and Dbh-negative neurons). In some embodiments, the stimulus can be identified as a selective modulator of the immune response (e.g., when the stimulus activates the neurons of interest by has no or minimal effect on non-target neurons).

Measuring the activation of neurons can be accomplished by a variety of methods. For example, activation of neurons can be performed by assessment of c-fos induction, targeted recombination in active populations (TRAP), or by detecting calcium response. In some embodiments, measuring the activation comprises detecting calcium response. Measurement of these relevant features can be accomplished by any suitable method, such as, for example, in vivo imaging (e.g., when a calcium indicator is used), immunohistochemistry (e.g., for measuring c-fos induction), or other methods.

The screening method can comprise a mammal (e.g., any of the model animals described herein) expressing a genetically encoded calcium indicator (GECI) in the relevant neurons of the mammal's brain. In some embodiments, the GECI is expressed in the nodose ganglion region of the mammal's brain. In some embodiments, the GECI is expressed in the cNST of the mammal's brain. The GECI can be any known in the art and suitable to assess the activation of the relevant neurons in the nodose ganglion including but limited to GCaMP. Signal from the GECI can comprise florescence, luminescence, or any other means to determine neuron activation.

Any therapeutic agent can be assessed for its ability to alter an immune response via administration through a variety of routes, including but not limited to, oral, intraperitoneal, subcutaneous, or intravascular. Therapeutic agents can encompass any substance with potential benefits to the mammal after administration which can include but are not limited to small molecule compounds, antibodies, a proteins, nucleic acids, or any combination thereof.

In some embodiments, the screening method can further comprise measuring additional signals of a modulated immune response, such as, for example, expression or changes in expression of pro-inflammatory cytokines, expression or changes in expression of anti-inflammatory cytokines, or other measurements associated with the immune response. In some embodiments, the method can further comprise assessing the change in effect of the immune response to an immune reacting inducing event due to the stimulus. For example, measurement of the change in cytokine levels in response to challenge with LPS can also be performed and compared between stimulus treated subjects and control subjects.

### EXAMPLES

### Example 1

A brainstem circuit that functions as a conduit to convey peripheral immune information to the brain was identified. Activation of this circuit can trigger robust suppression of peripheral immune activity. Using an activity marker (cFos induction), it was determined that a selective population of neurons in the brain are activated in response to administration of a peripheral immune insult.

An immune insult with LPS triggers strong innate immune responses, characterized by the release of proinflammatory cytokines like IL-1b, IL-6 and TNFa, and the release of anti-inflammatory cytokines, like IL-10, that help modulate and dampen the inflammatory response. A balanced ratio of pro- and anti-inflammatory cytokines is important for an appropriate immune response.

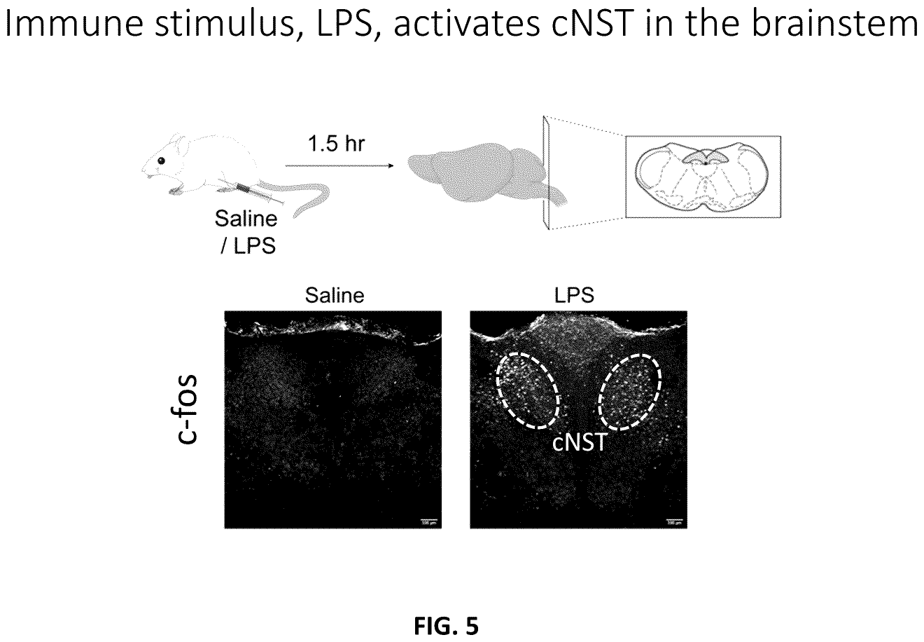

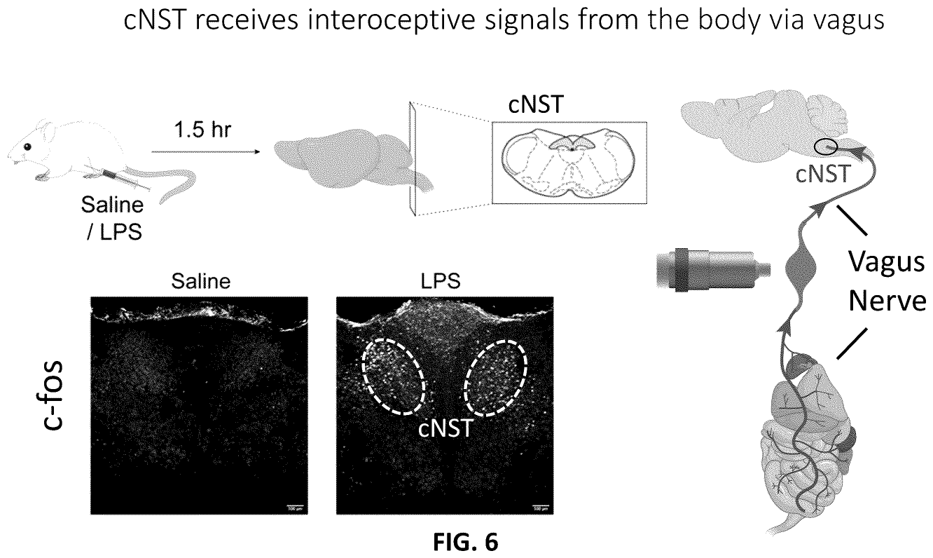

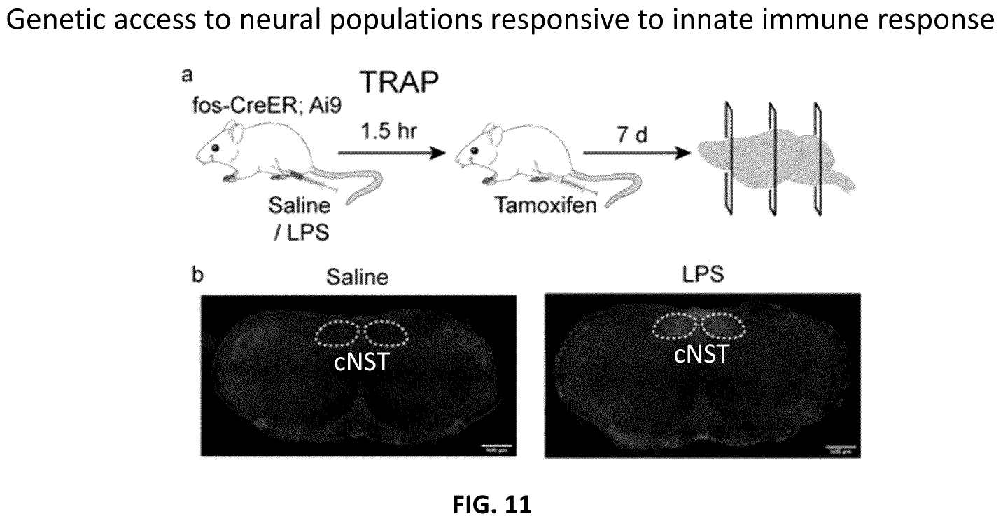

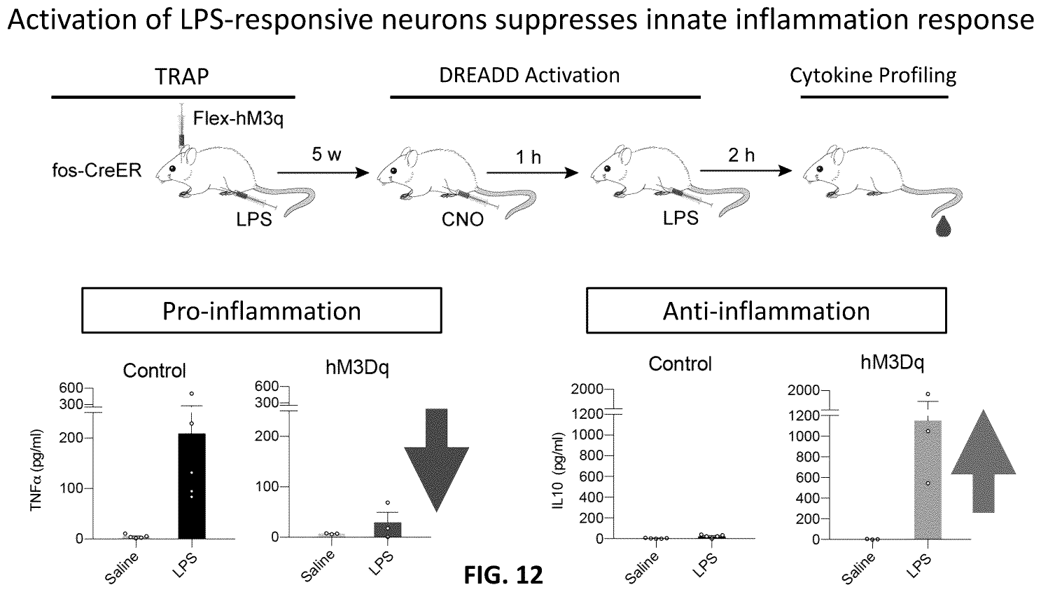

It was shown that neurons in the caudal nucleus of the solitary tract (cNST) in the brainstem are activated by peripheral (intraperitoneal) administration of LPS (lipopolysaccharide), a potent inducer of inflammatory responses (FIG. 5). An activity-based labeling strategy (targeted recombination in active populations, TRAP) was employed to genetically access the LPS-activated neurons in the cNST (FIG. 11), and demonstrated that chemogenetic activation of these LPS-TRAPed cNST neurons robustly suppresses LPS-evoked the release of pro-inflammatory cytokines (e.g., TNFa) while enhancing the release of anti-inflammatory cytokines (e.g., IL10) (FIG. 12). In other words, the brain monitors and modulates immune responses in the body. These results open up unique opportunities to significantly modulate immune/inflammatory responses by acting on the nervous system. Next, to uncover the role of these cNST neurons in immune responses, a chemogenetic silencer was introduced by genetic Trapping, and the effect of silencing these cNST neurons in the emergence of LPS-induced inflammatory responses was evaluated.

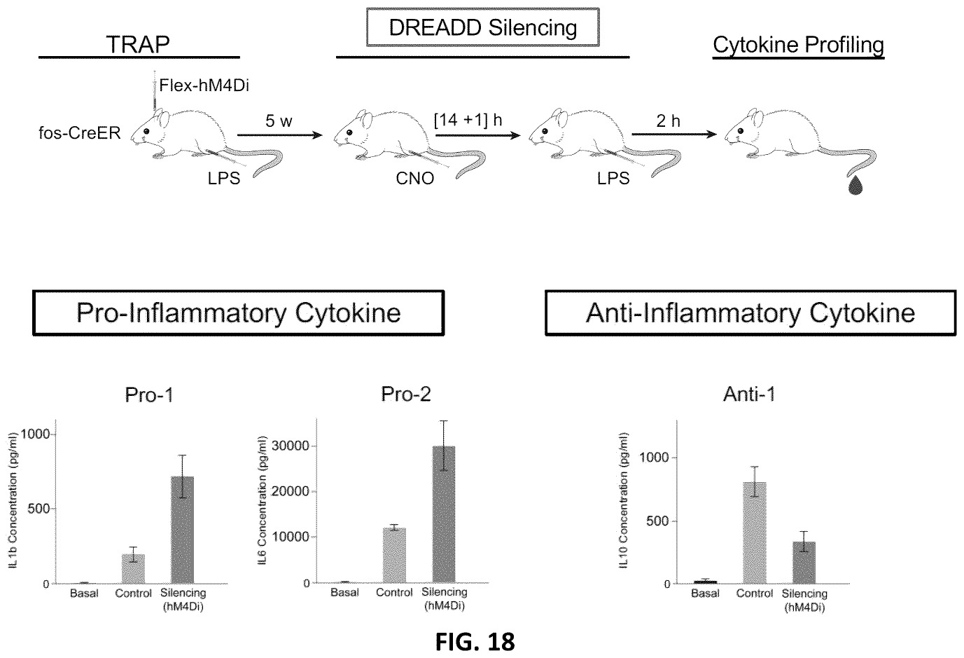

In a normal response, LPS triggers the release of proinflammatory and anti-inflammatory cytokines, resulting in a balanced and controlled immune reaction. However, when this LPS-activated cNST neurons are silenced, there is a complete change in the inflammatory response: an out-of-control increase in the release of proinflammatory cytokines and a dramatic dampening in the release of anti-inflammatory cytokines (FIG. 18).

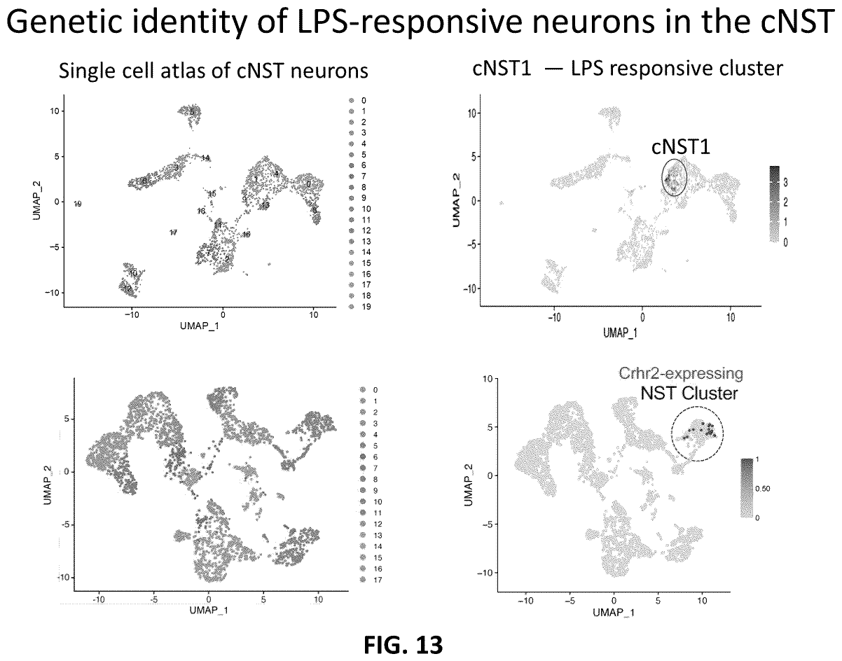

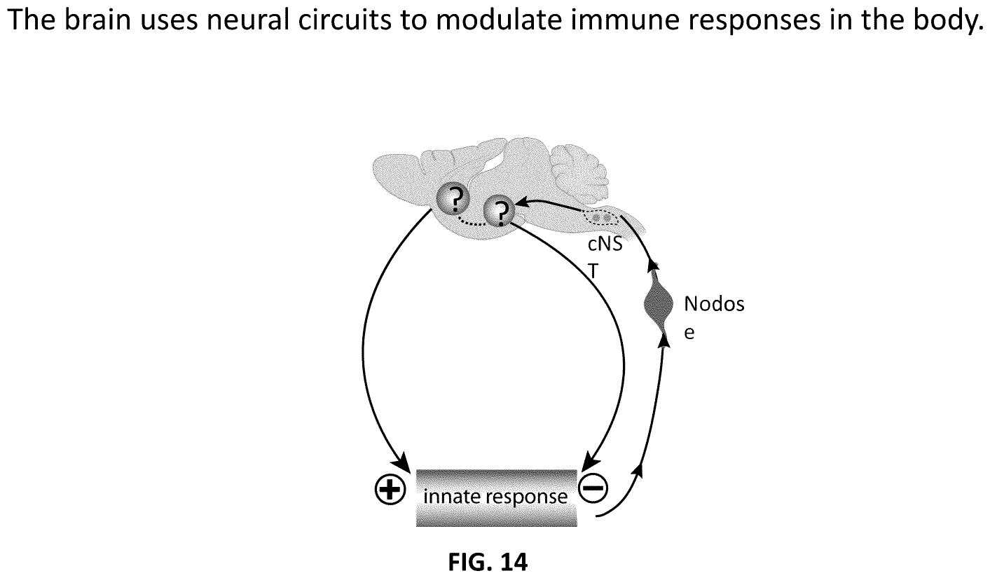

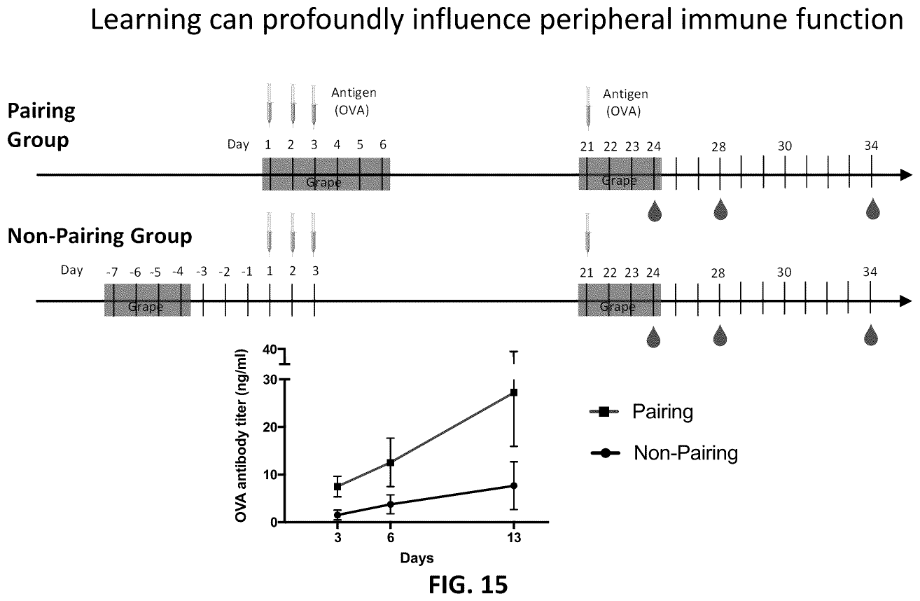

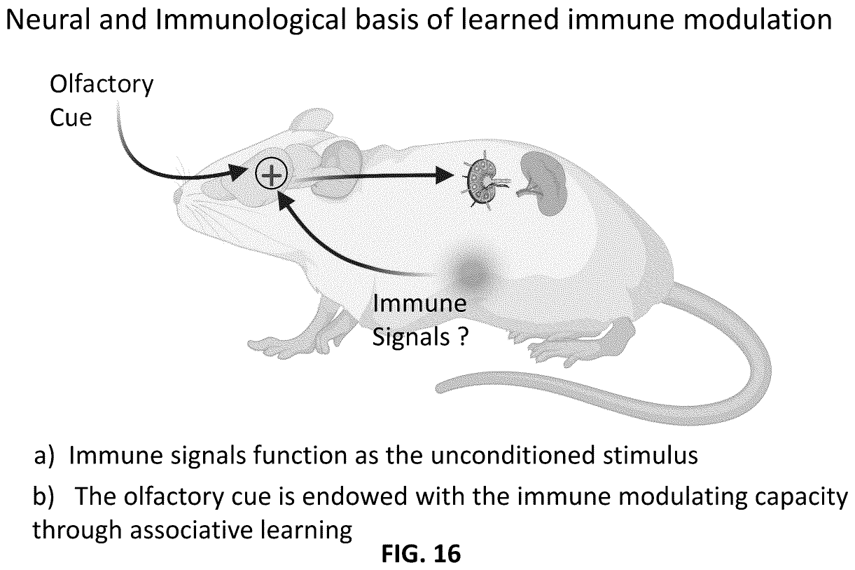

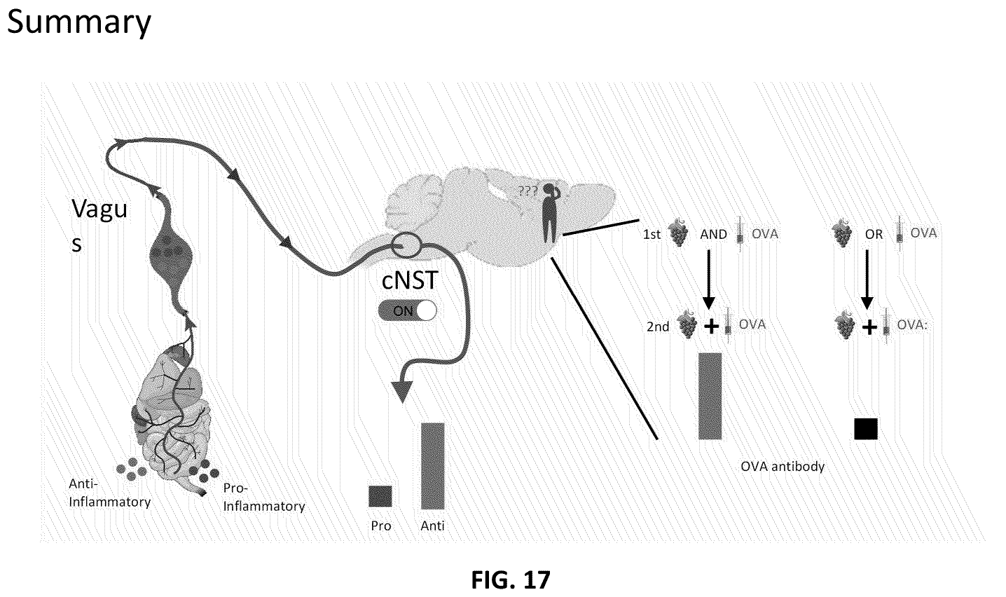

A single cell atlas of the cNST was generated and ACT-Seq was used to identify the LPS-activated neurons: Corticotropin releasing hormone receptor-2 expressing neurons. A Cre driver line was then generated to manipulate their function, a chemogenetic activator was introduced, and it was shown that activating this unique, genetically identified cNST population, is sufficient to modulate the course of the inflammatory responses (FIG. 13).

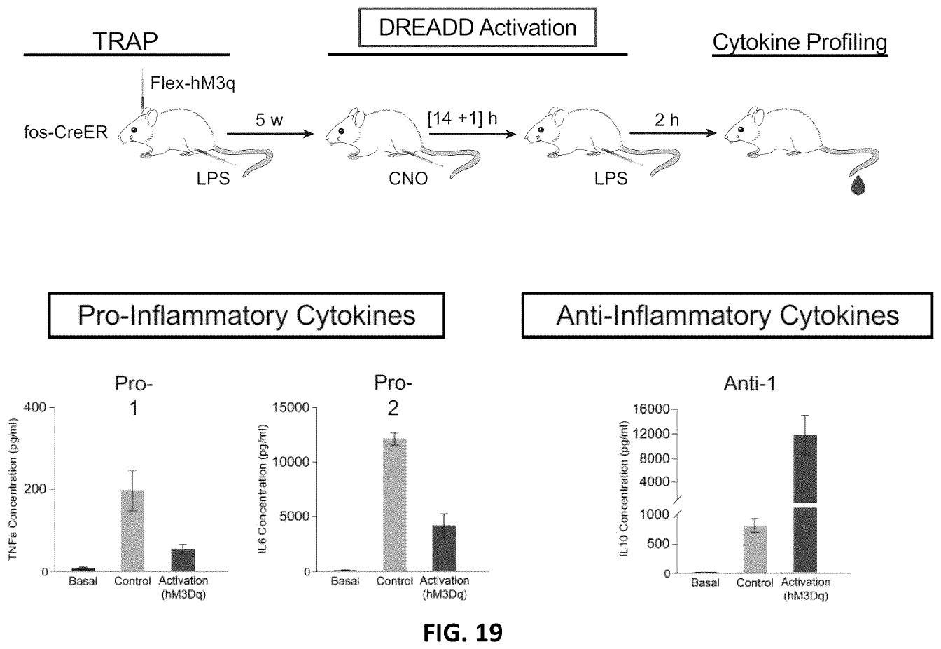

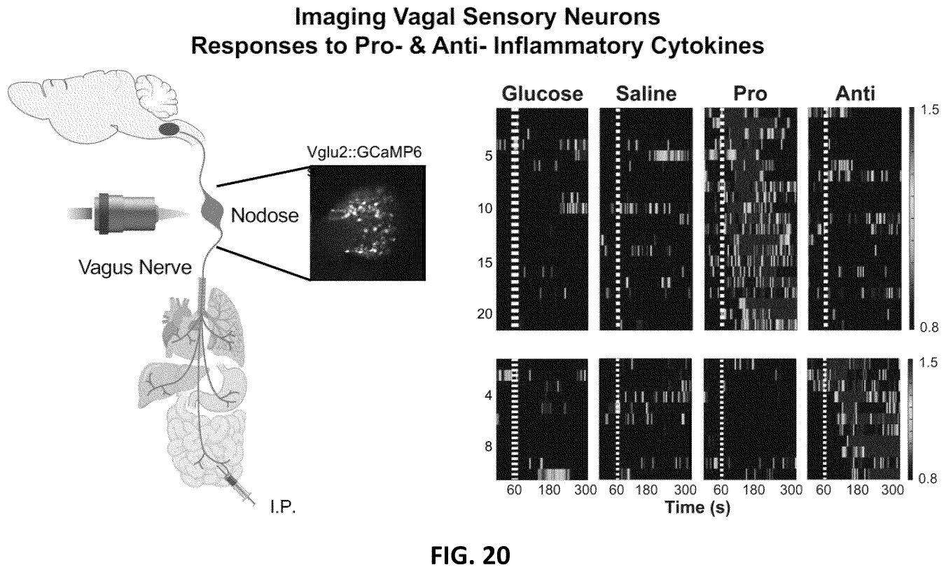

The TRAP system was used to gain genetic access to these neurons, and selectively activating them chemogenetically. The effect of activation after LPS injection could then be examined. Activating these neurons leads to a dramatic suppression of the release of pro-inflammatory cytokines, accompanied with a very large increase in the levels of anti-inflammatory cytokines, in essence a transformation of the inflammatory response (FIG. 19). These results provide a powerful strategy to suppress inflammatory responses and the inflammatory state. This strategy could be deployed to help manage autoimmune disorders, cytokine storm responses, graft-vs-host reactions, cancer immunotherapies, toxic shock syndrome, etc.

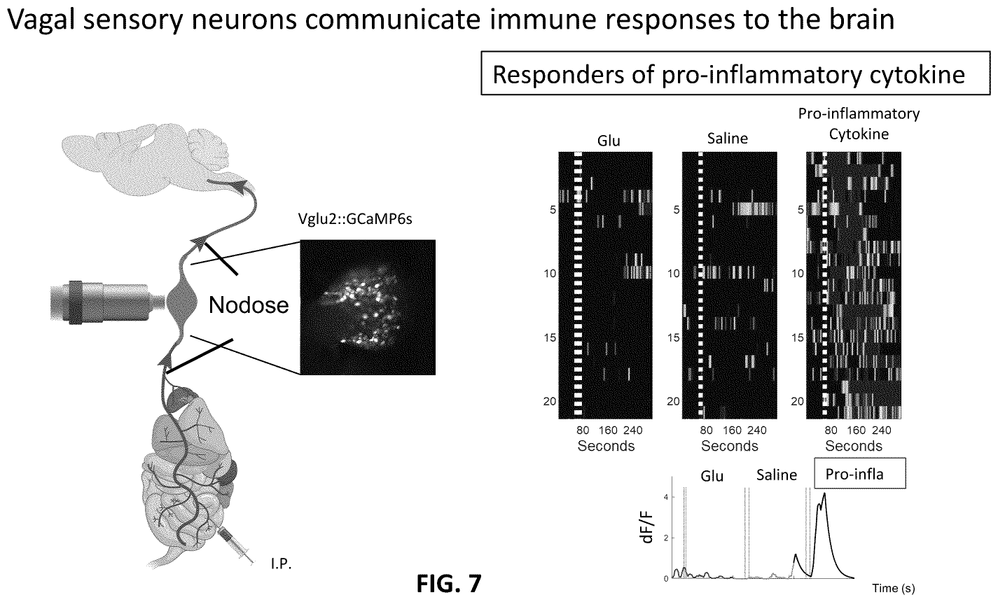

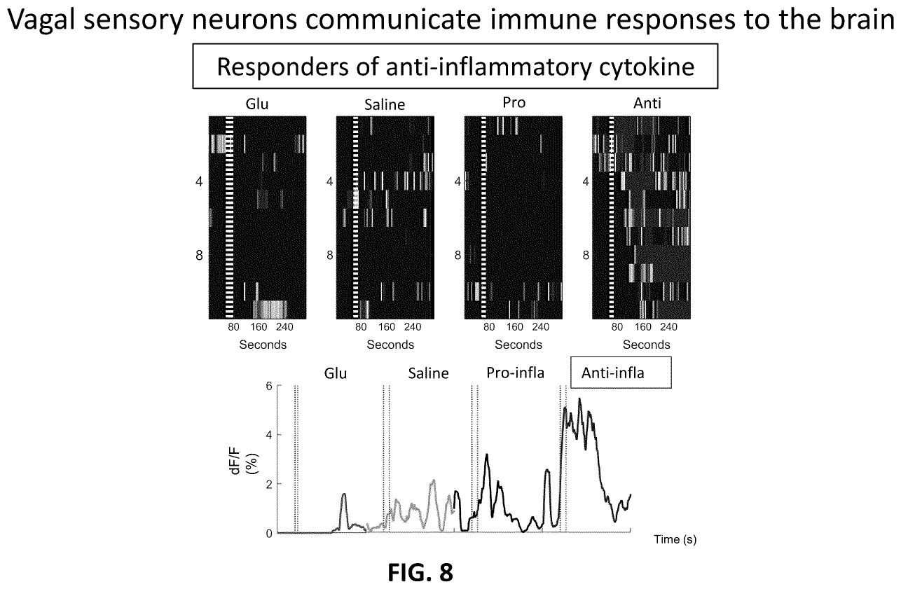

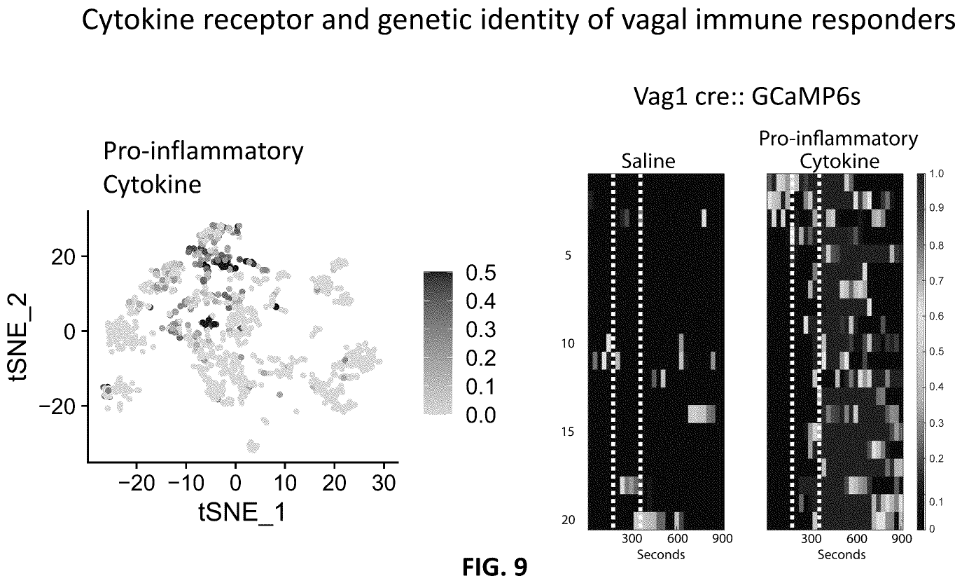

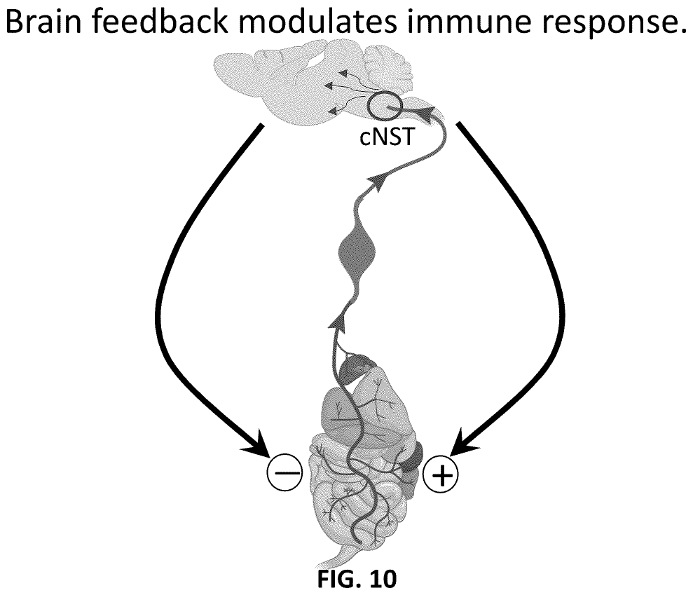

One of the main pathways of information between the body and the brain is the vagal nerve, thus vagal sensory neurons activation in response to immune signals was analyzed. Using an imaging platform to record activity from vagal ganglia, it was shown that a selective subset of vagal sensory neurons respond robustly and reliably to administration of proinflammatory cytokines (e.g., TNFa & IL1b) (FIG. 7), and further demonstrated that this population of neurons is defined by the expression of the vasoactive intestinal peptide (VIP) gene (FIG. 9). Importantly, a separate population of vagal neurons that are activated by stimulation with anti-inflammatory cytokines (e.g., IL4; FIG. 8) was also identified.

Given that vagal sensory neurons transmit information to the brain by synapsing with targets in the caudal nucleus of the solitary tract (cNST) in the brainstem (see FIG. 5), the results provide a direct path to modulate this brain circuit either peripherally (i.e., via vagal neurons) or centrally (i.e., via cNST neurons).

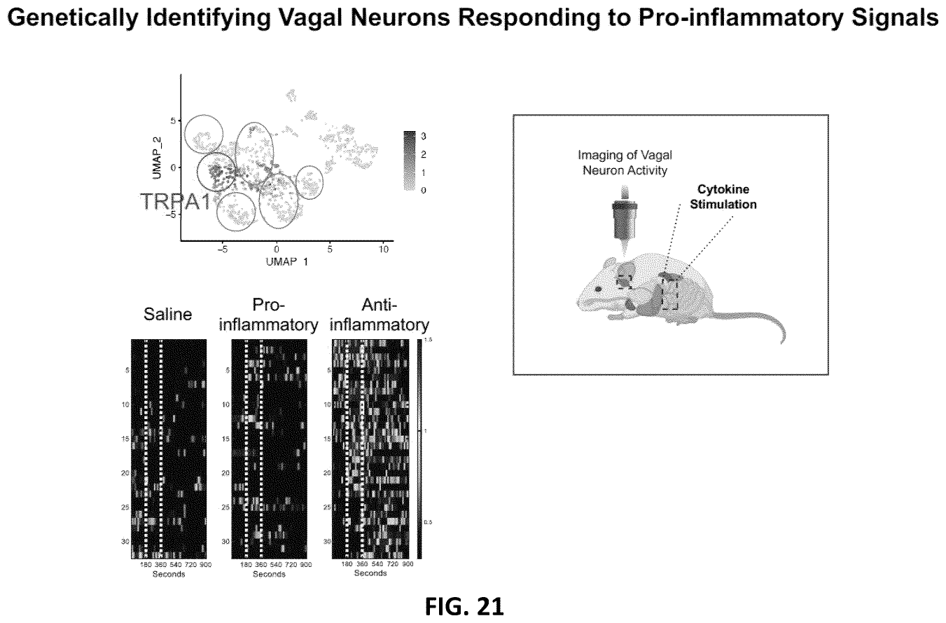

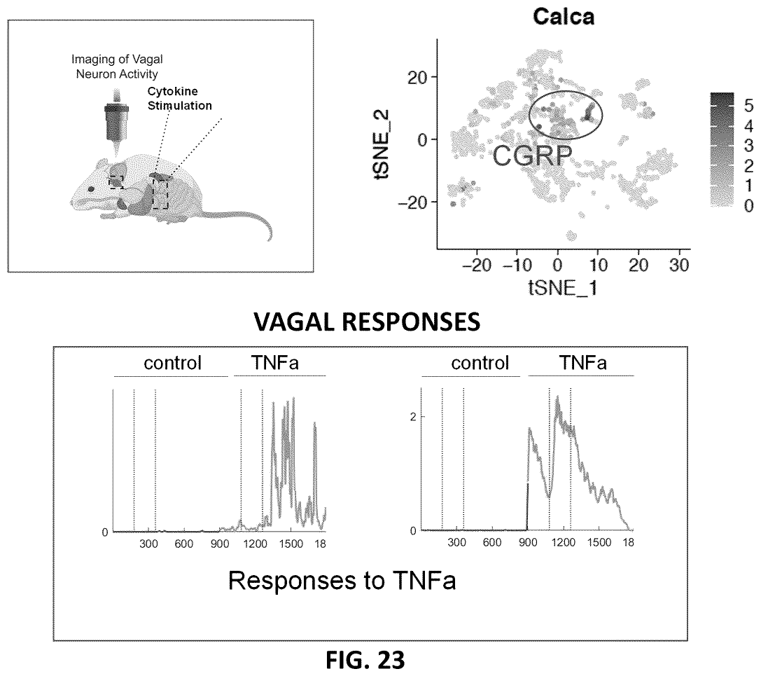

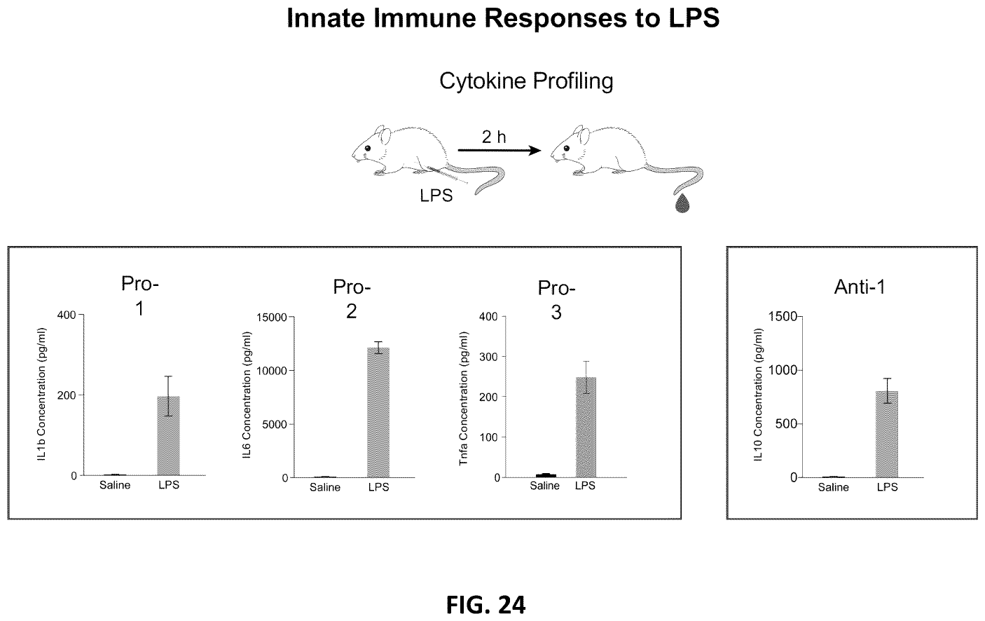

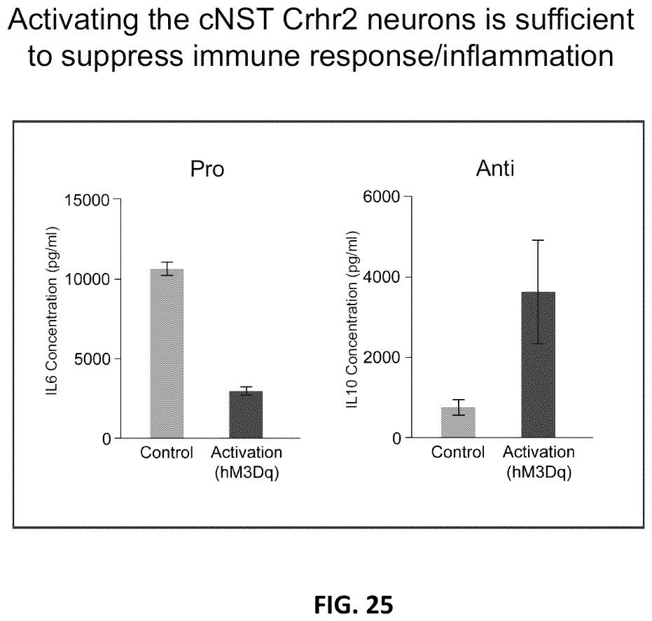

The single cell atlas of the vagal ganglia (Nodose) was used, combined with functional imaging to demonstrate that TrpA1-expressing Neuron in the vagal ganglia respond to anti-inflammatory signals (FIG. 21). Further, single cell atlas of the vagal ganglia (Nodose), combined with functional imaging demonstrate that the Calca-expressing (calcitonin gene-related peptide—CGRP) neurons in the vagal ganglia carry TNFa pro-inflammatory signals from the body to the brain (FIG. 23).

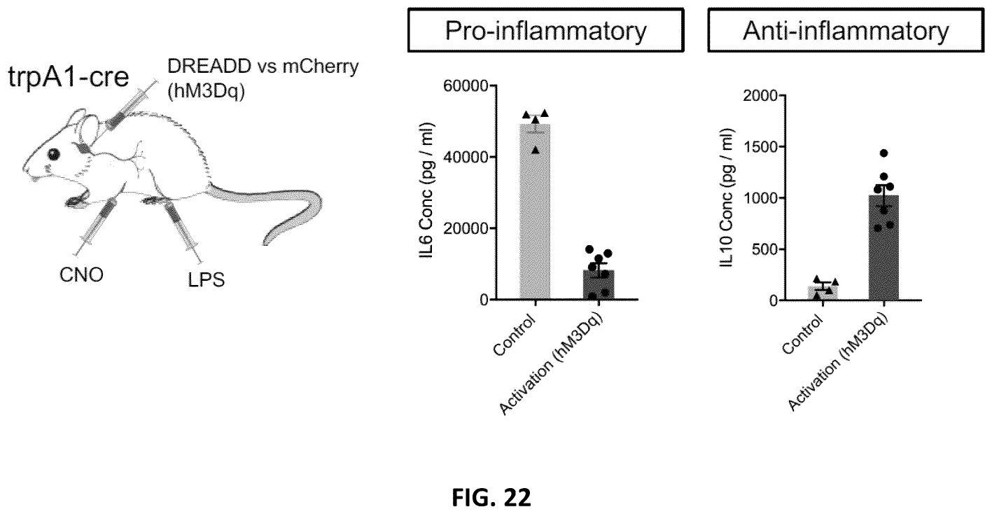

The TRPA1-expressing neuron in the vagal ganglia trigger the brain modulation of the immune response, and their artificial activation (chemogenetically in this example) mimic the effect of activating the cNST neurons, and lead to the suppression of pro-inflammatory cytokines, and the induction of anti-inflammatory ones (FIG. 22).

Immune responses are a critical point of physiological modulation in a wide range of treatments, diseases and disorders. The ability to bias immune responses (i.e., suppress or enhance) via neural circuits provides exciting novel therapeutic opportunities. The disclosed work provides a novel platform (i.e., imaging of nodose ganglion) to screen small molecules that can be administered peripherally to alter immune responses. The identification of genetically defined populations in the nodose provides candidates for pharmacologically targeted interventions. Interventions may also act centrally at the cNST by using an approach to target specifically the neurons identified in the studies that mediate top-down immune modulation.

FIG. 5. demonstrates that neurons in the caudal nucleus of the solitary tract are activated upon immune stimulation with the known immunostimulant LPS. This finding illustrates a direct neuronal response to immune system activation in a specific area of the brain. Importantly, FIG. 12. demonstrates this pathway is bidirectional and that direct stimulation of neurons in the caudal nucleus of the solitary tract suppresses secretion of the pro-inflammatory cytokine TNFa after LPS stimulation. Relatedly, following activation of the caudal nucleus of the solitary tract, the anti-inflammatory cytokine IL-10 is secreted in greater quantities after LPS stimulation. This establishes that activation of neurons in the caudal nucleus of the solitary tract is a novel method to regulate immune responses.

FIGS. 7-9. demonstrate that subsets of neurons in the nodose ganglion respond to either pro-inflammatory cytokines or anti-inflammatory cytokines through activation. These neuronal subsets can be used as a readout to assess the effect of different therapeutic agents on immune system activation. Using the GECI system described, immune system activation or suppression from a therapeutic agent can be determined via a florescence or luminescence readout from neurons that respond to either pro-inflammatory signals or neurons that respond to anti-inflammatory signals.

### Example 2

### Brainstem Neurons Activated by Innate Immune Responses

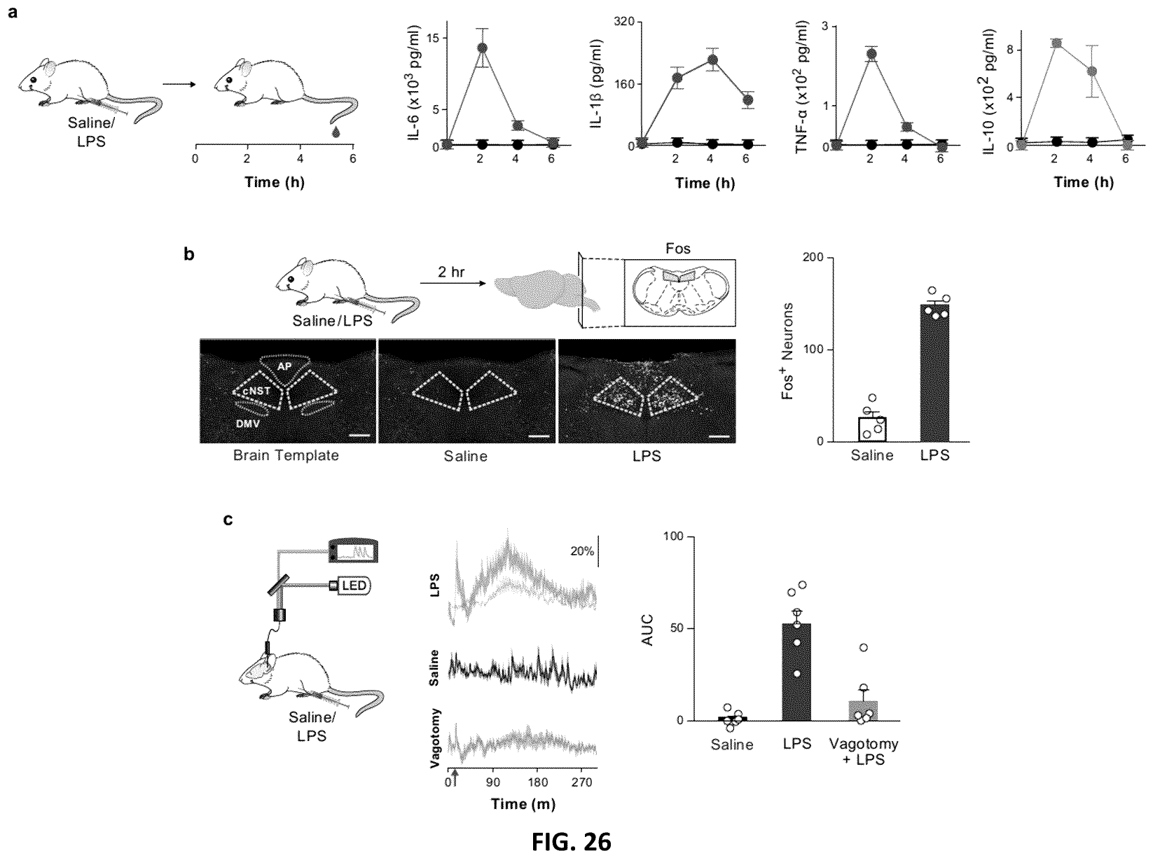

Lipopolysaccahride (LPS), a canonical immune stimulus derived from the outer membrane of gram-negative bacteria was used to elicit innate immune responses. Separate cohorts of mice were challenged with intraperitoneal (IP) injection of LPS and vehicle control (saline), and then the evoked immune response was examined by measuring cytokine changes in peripheral blood samples. A single dose of LPS is sufficient to trigger significant increases in pro-inflammatory and anti-inflammatory cytokines, with a time course peaking at about 2 hours post LPS injection (FIG. 26 Panel a). Next, the animals' brains were scanned for induction of the immediate early gene Fos as a proxy for neural activity. The results showed stimulus-evoked labeling in the Area Postrema (AP) and strong labeling in the caudal Nucleus of the Solitary Tract (cNST) in the brainstem (FIGS. 26 Panel b and 32); minor labeling was observed in response to control saline injections. The AP is known to be activated by body malaise, and hence it would be expected to exhibit some labeling. The cNST, on the other hand, is the primary target of the vagus nerve, and it functions as the major conduit in the body-brain axis.

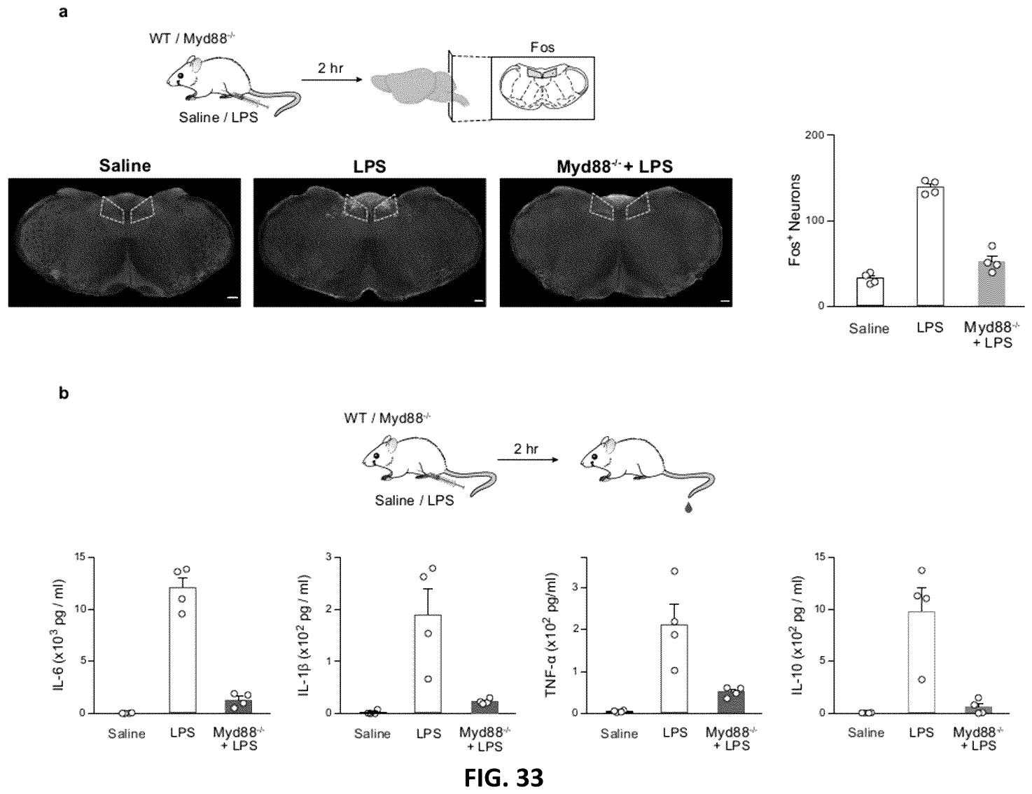

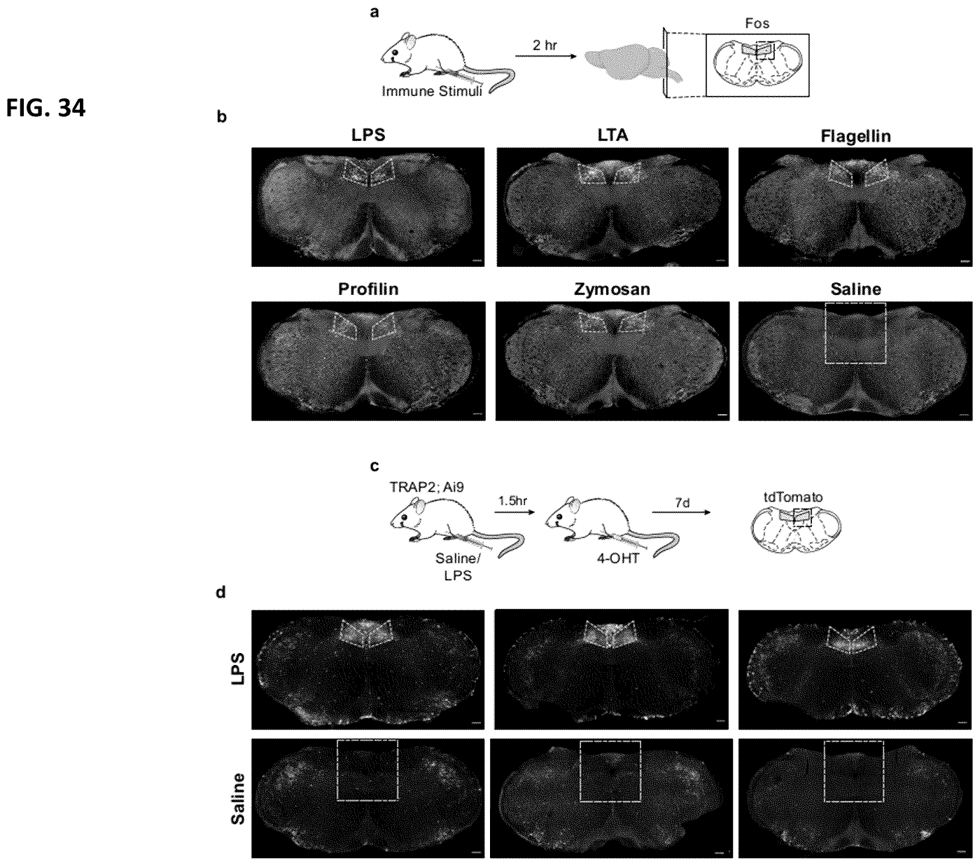

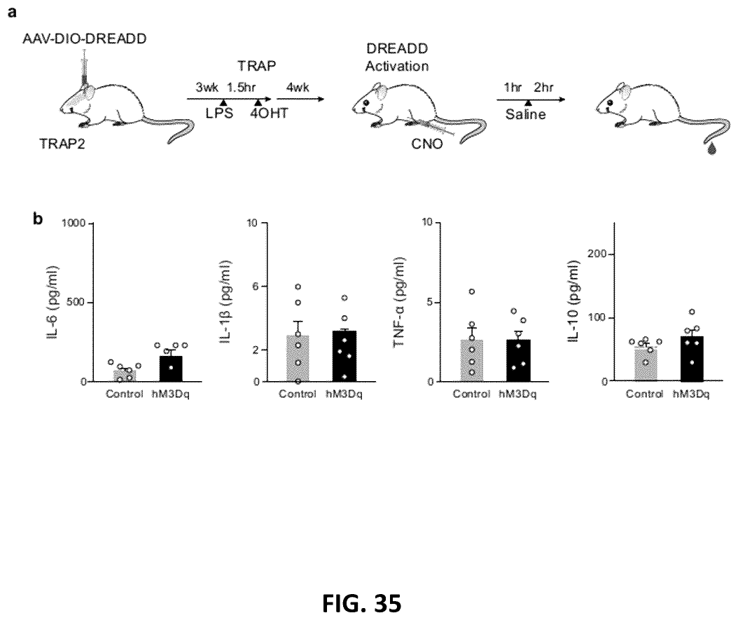

Injections of LPS in animals homozygous knockout for Myd88 (an essential component of the LPS receptor in immune cells) do not activate cNST neurons (FIG. 33) showing that LPS stimulates cNST labeling via its action on immune cells. Robust cNST labeling was also observed in response to a variety of other immune insults (FIG. 34 Panels a and b).

To directly monitor the activation of cNST neurons following the peripheral LPS challenge, cNST neurons were targeted with an AAV harboring a GCaMP6s construct, so as to drive expression of the activity reporter in cNST neurons, and responses were recorded in awake behaving animals using fiber photometry (FIG. 26 Panel c). The results demonstrated cNST activation that tracks the emergence and the development of the innate immune response (compare FIG. 26 Panel c with FIG. 26 Panel a).

Bilateral transection of the vagus nerve eliminates cNST responses to LPS (FIG. 26 Panel c). These results substantiate the vagal-cNST immune axis, and demonstrate that the LPS-evoked activity is not the result of LPS directly accessing cNST neurons.

### Example 3

Silencing LPS-Responsive cNST Neurons Transforms the Immune Response

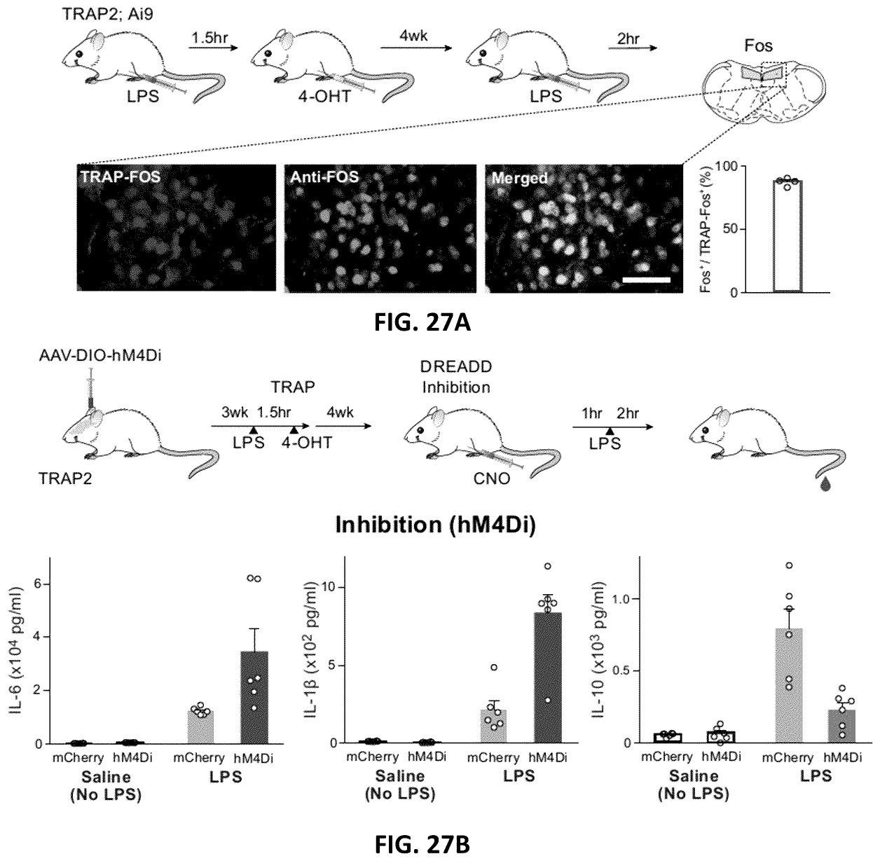

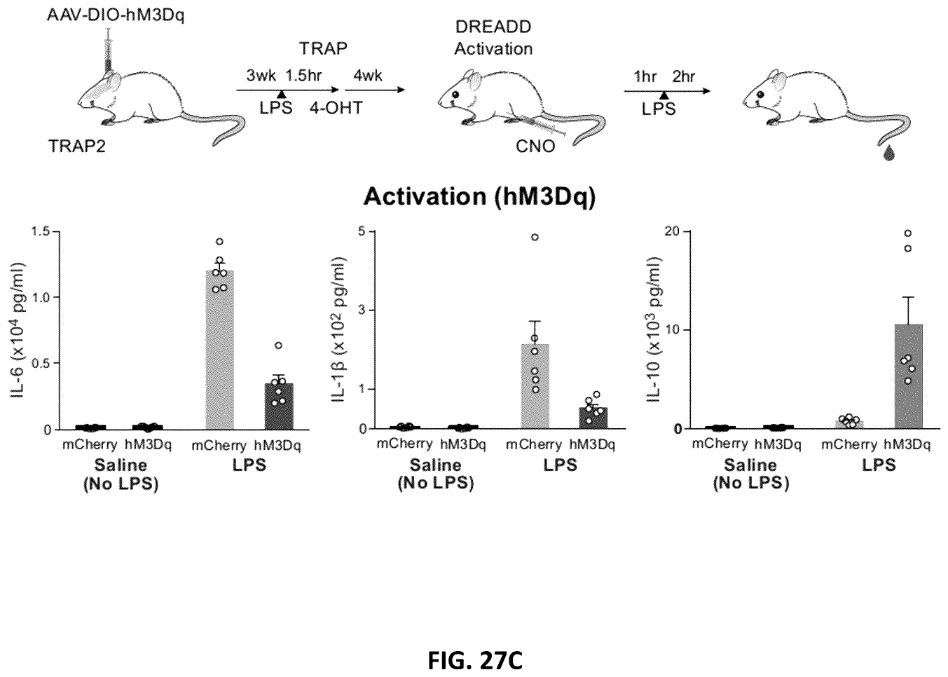

Targeted recombination in active populations (TRAP) system was used to target Cre-recombinase to the LPS-activated neurons (FIG. 34 Panels c and d), and a Cre-dependent genetic silencer was used to examine LPS-evoked responses in control and silenced animals. First, to monitor the fidelity of the TRAP strategy it was confirmed that the LPS-activated cNST neurons marked by the expression of Fos are the same as the ones labeled by Cre-recombinase in the genetic TRAPing experiments. The LPS-induced TRAPed neurons were genetically labeled with a Cre-dependent tdTomato reporter, and a second cycle of LPS stimulation followed by Fos antibody labeling was then performed. The results confirmed that the majority (>80%) of the LPS-TRAPed neurons (e.g., labeled with tdTomato in the cNST) were indeed co-labeled with the Fos antibodies in response to the second cycle of LPS stimulation (FIG. 27A).

Next, the cNST of LPS-TRAPed animals was bilaterally injected with an AAV virus carrying a Cre-dependent inhibitory DREADD (iDREADD). Hence, the TRAPed LPS-activated neurons would turn-on Cre-recombinase and enable expression of the Cre-dependent iDREADD, thus allowing chemogenetic inhibition of those cells. The iDREADD-expressing animals were then challenged with LPS, and the resulting immune response were monitored (FIG. 27B, upper panel). Remarkably, chemogenetic inhibition of the cNST neurons resulted in a dramatic increase in the pro-inflammatory response, and a concomitant decrease of the anti-inflammatory response (FIG. 27B, lower panels). In essence, a run-away, out-of-control inflammatory response. Indeed, the levels of pro-inflammatory cytokines rise to over 300% compared to the levels observed in LPS-treated but not silenced animals (for example IL-1β goes from 200 μg/ml to 800 μg/ml; FIG. 27B), while the anti-inflammatory component exhibited a profound reduction (IL-10 levels are reduced from 750 μg/ml to ˜250 pg/ml, FIG. 27B). These results suggest the cNST functions as a homeostatic neural control of peripheral immune responses.

### Example 4

Activation of LPS-Responsive Neurons in the cNST Inhibits Inflammatory Responses

The TRAP system was used to virally target an excitatory, rather than inhibitory, DREADD (hM3Dq) to the LPS-activated neurons, and the impact of activation of this circuit on the LPS-evoked inflammatory response was tested. Chemogenetic activation of the LPS-TRAPed neurons inhibited the pro-inflammatory response while substantially increasing the anti-inflammatory response. As shown in FIGS. 27A-C, pro-inflammatory cytokines are reduced nearly 70% from the levels observed in the control LPS-evoked responses, while anti-inflammatory levels are up nearly 10-fold. Together, these silencing and activation experiments demonstrate that modulating the activity of these brainstem neurons can bidirectionally regulate peripheral inflammation. Importantly, activating this circuit in the absence of an immune challenge has no effect on cytokine levels, validating its role in monitoring and regulating an immune response rather than initiating it (e.g., no LPS control in FIGS. 27 (Panel c) and 35).

### Example 5

cNST Neurons Suppress Inflammation

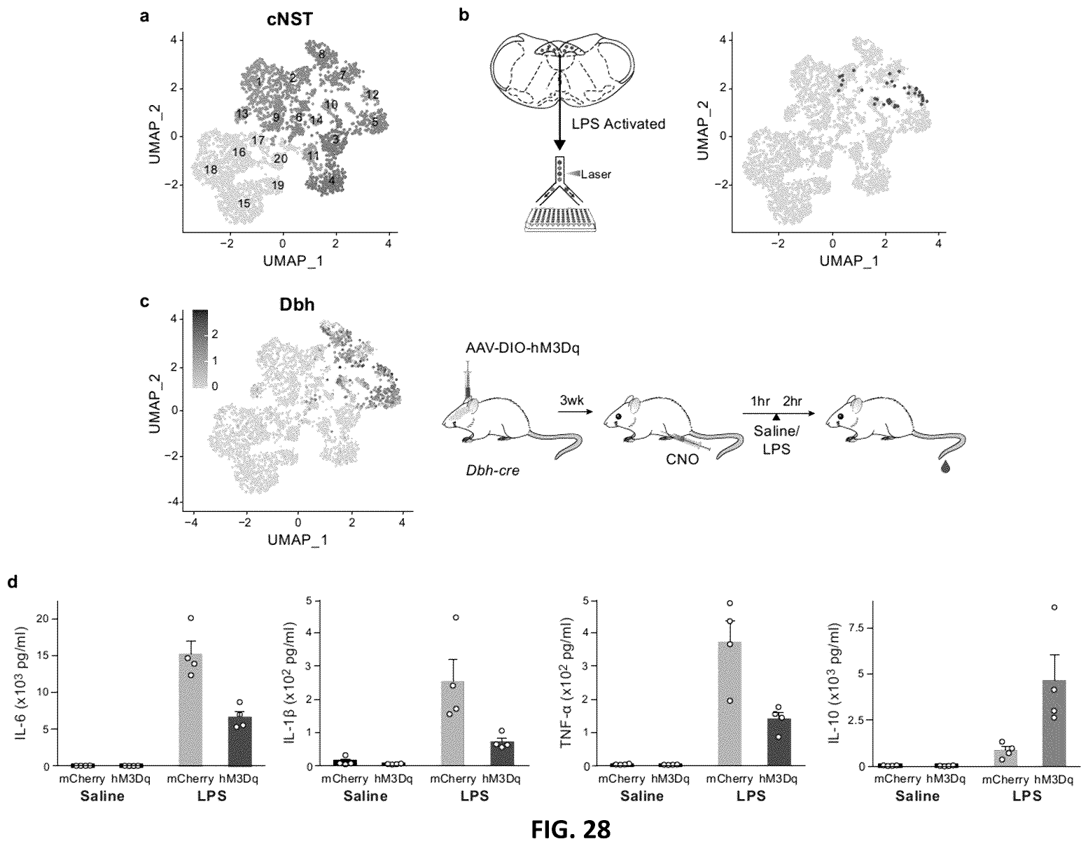

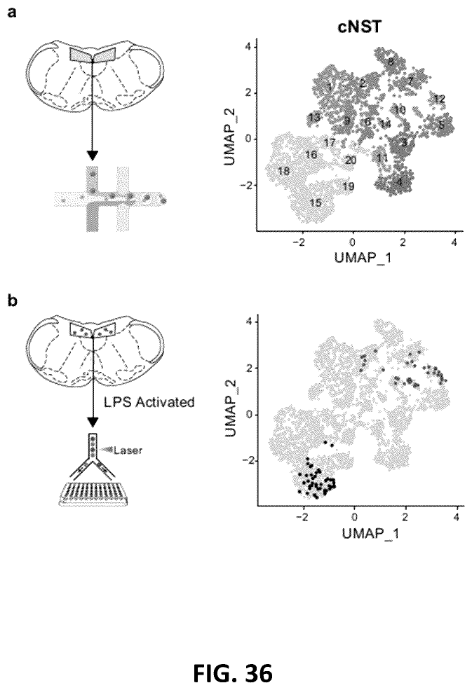

To identify the cNST neurons modulating inflammation, single-cell RNA sequencing (scRNA-seq) was performed on 4008 cells from the cNST (FIG. 28 Panel a). scRNA-seq was carried out on 288 individual neurons TRAPed with LPS (along with ˜100 unlabeled neurons), and LPS-TRAPed neurons are primarily found in 3 related glutamatergic clusters (clusters 7, 10, 12, with a small number in cluster 2) (FIG. 28 Panel b) and 1 GABAergic cluster (cluster 15) (FIG. 36).

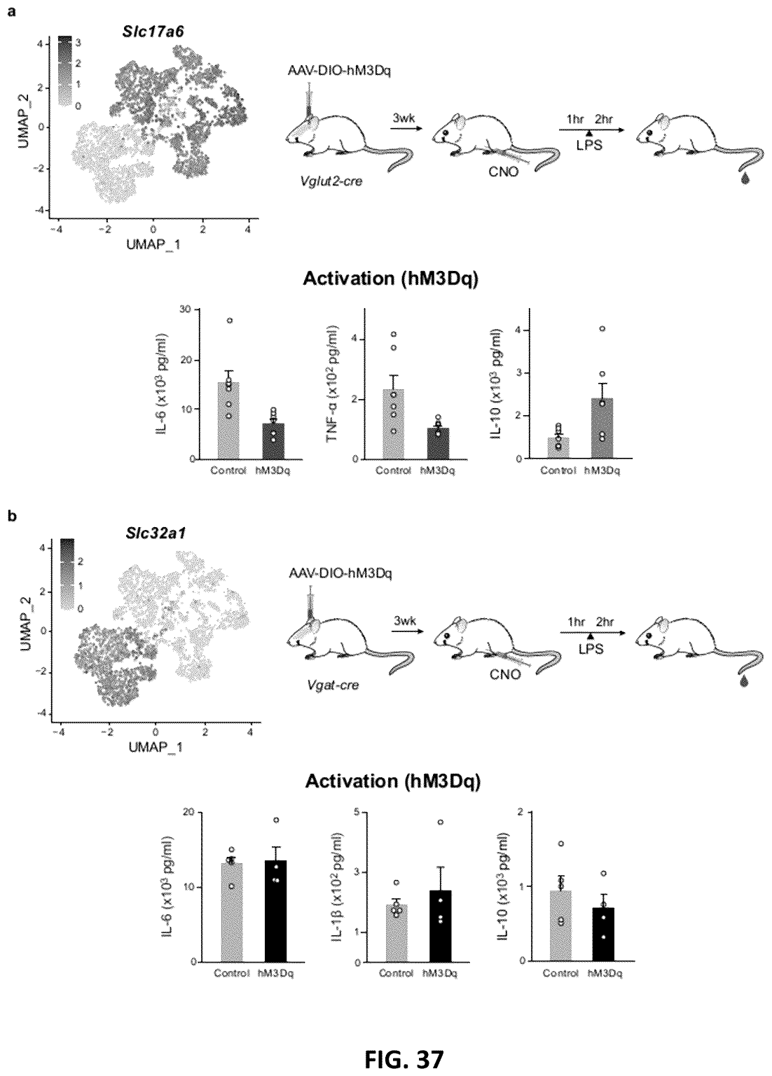

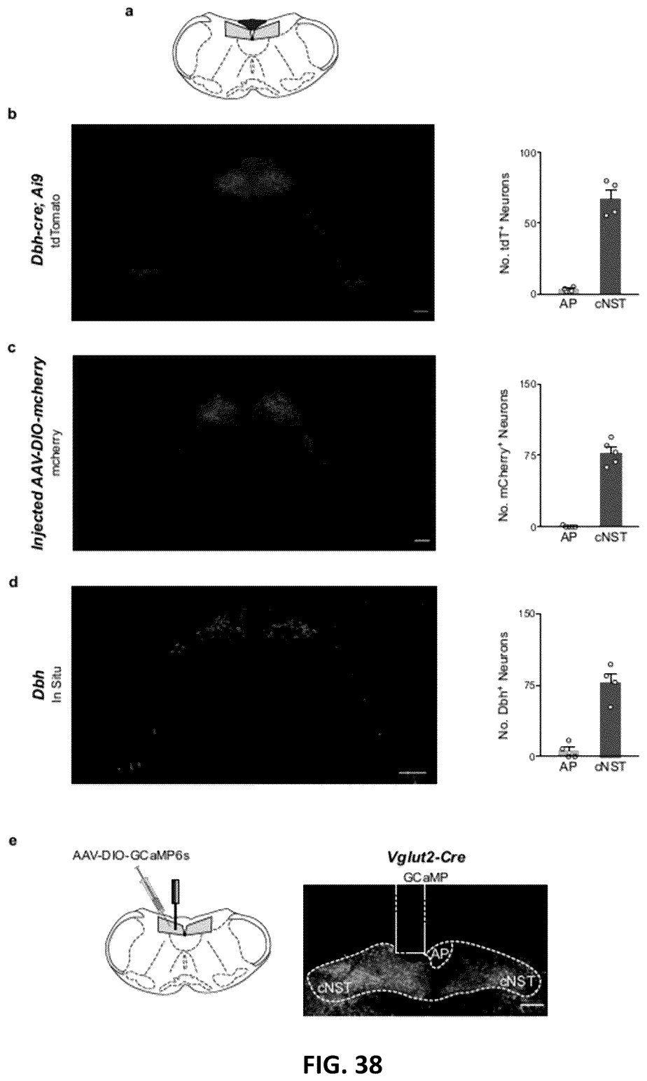

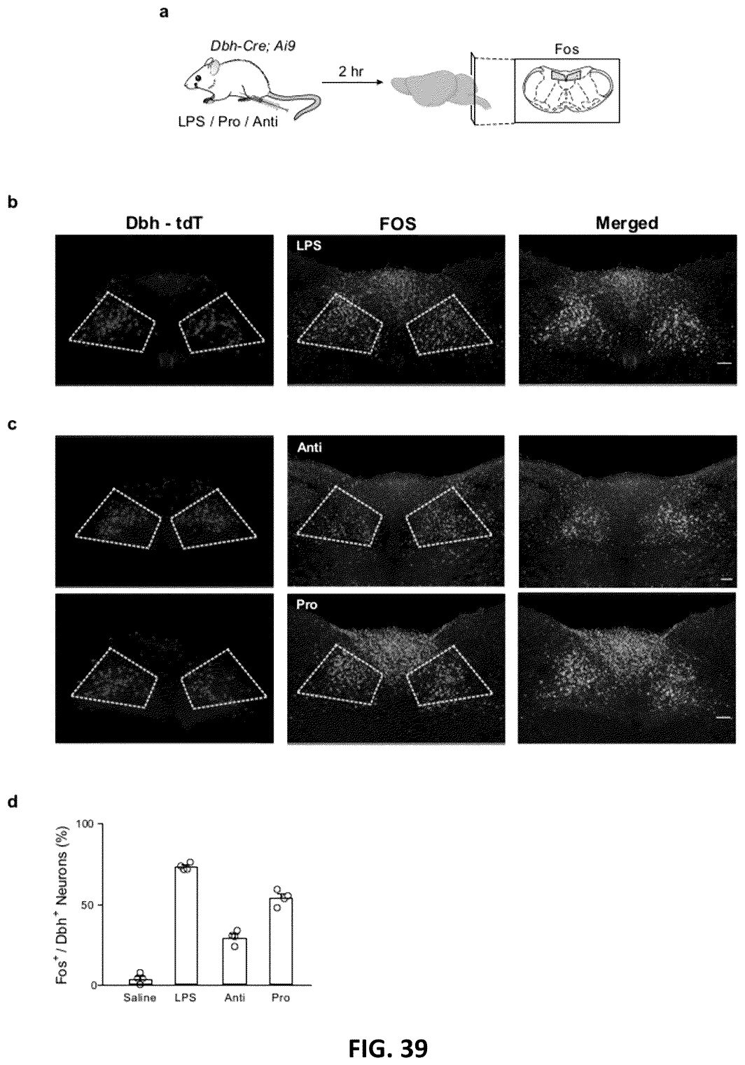

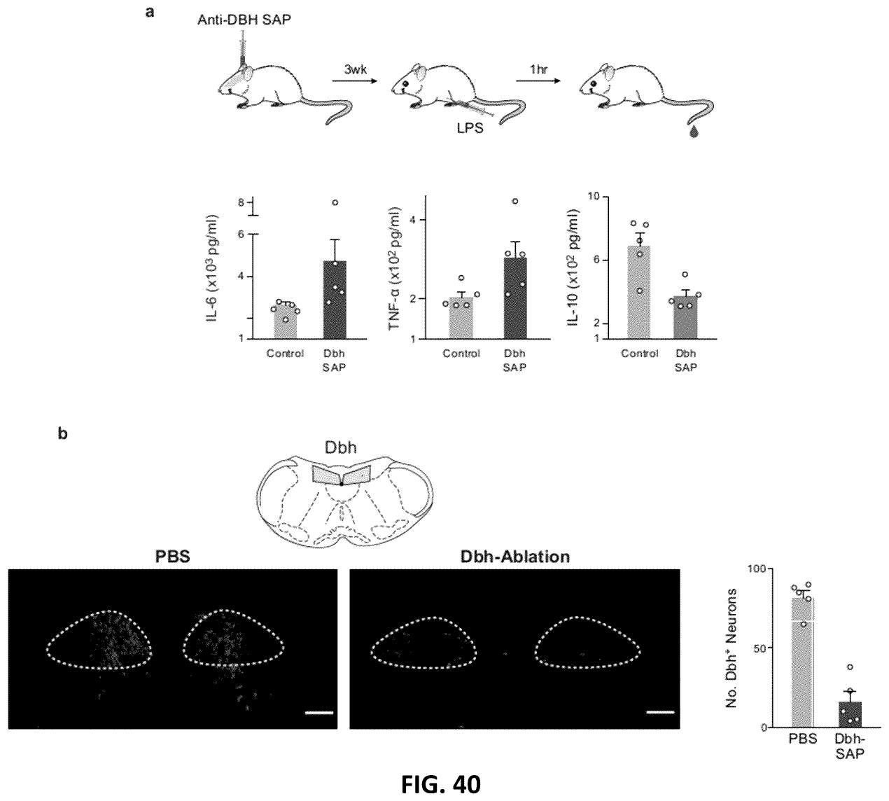

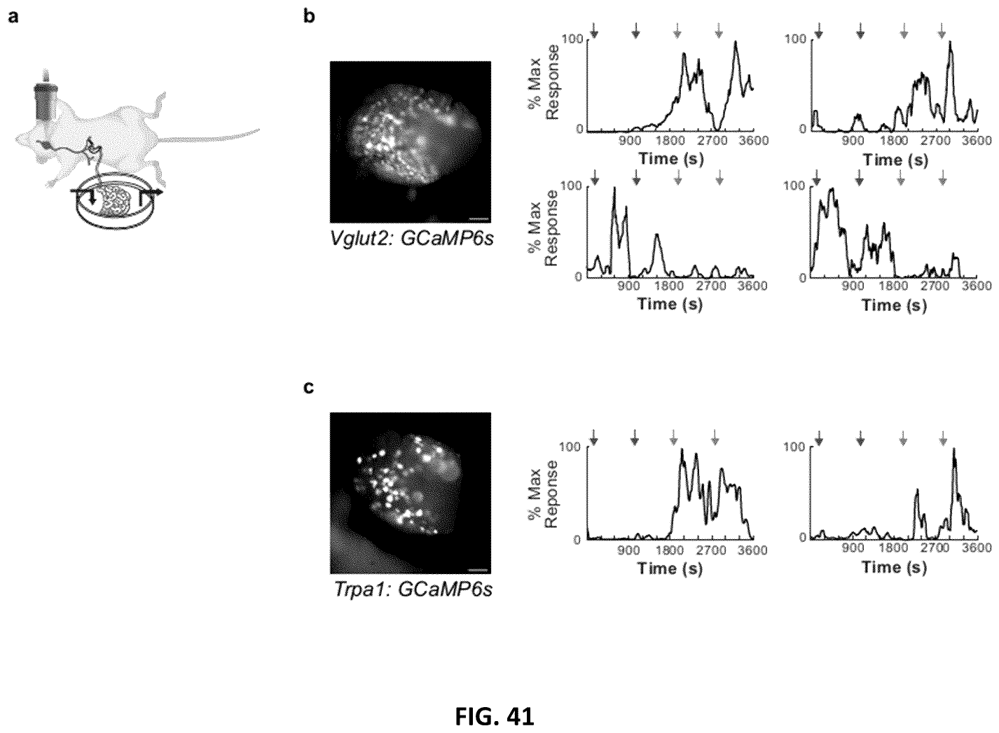

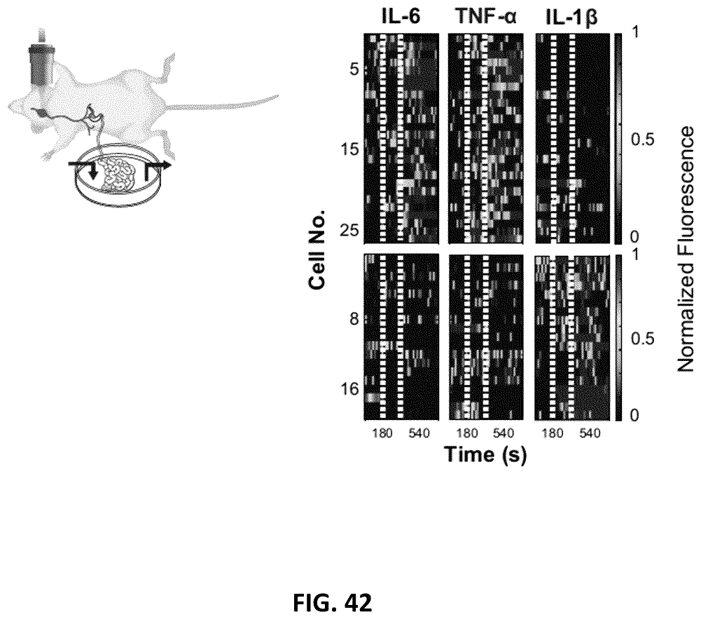

AAV viruses carrying a Cre-dependent excitatory DREADD were injected into the cNST of either Vglut2-cre or Vgat-cre mice. The results showed that activation of excitatory, but not inhibitory neurons, effectively suppressed LPS-induced inflammation, and largely mirrored the results obtained following activation of the LPS-TRAPed neurons (FIG. 37 Panel a); no effect was observed when activating GABAergic neurons (FIG. 37 Panel b). Next, clusters 7, 10 and 12 were screened for common, selectively expressed genes, and identified the Dopamine beta-hydroxylase (Dbh)₄₅ gene as a candidate marker (FIG. 28 Panel c). In contrast to previous reports, DBH expressing neurons in the brainstem are almost exclusively located in the cNST (see FIG. 38), and are strongly activated in response to LPS (FIG. 39). The cNST of Dbh-cre mice were targeted with an AAV encoding a Cre-dependent excitatory DREADD. Activation of DBH expressing neurons in the cNST markedly suppressed pro-inflammatory cytokines while greatly enhancing the anti-inflammatory IL-10 levels (FIG. 28 Panels c and d), demonstrating the ability of these cNST neurons to drive immune suppression. Next, the DBH+ neurons in the cNST were ablated and dysregulation of the immune response was observed (FIG. 40 Panels b and c).

### Example 6

### Vagal Responses to Immune Cytokines

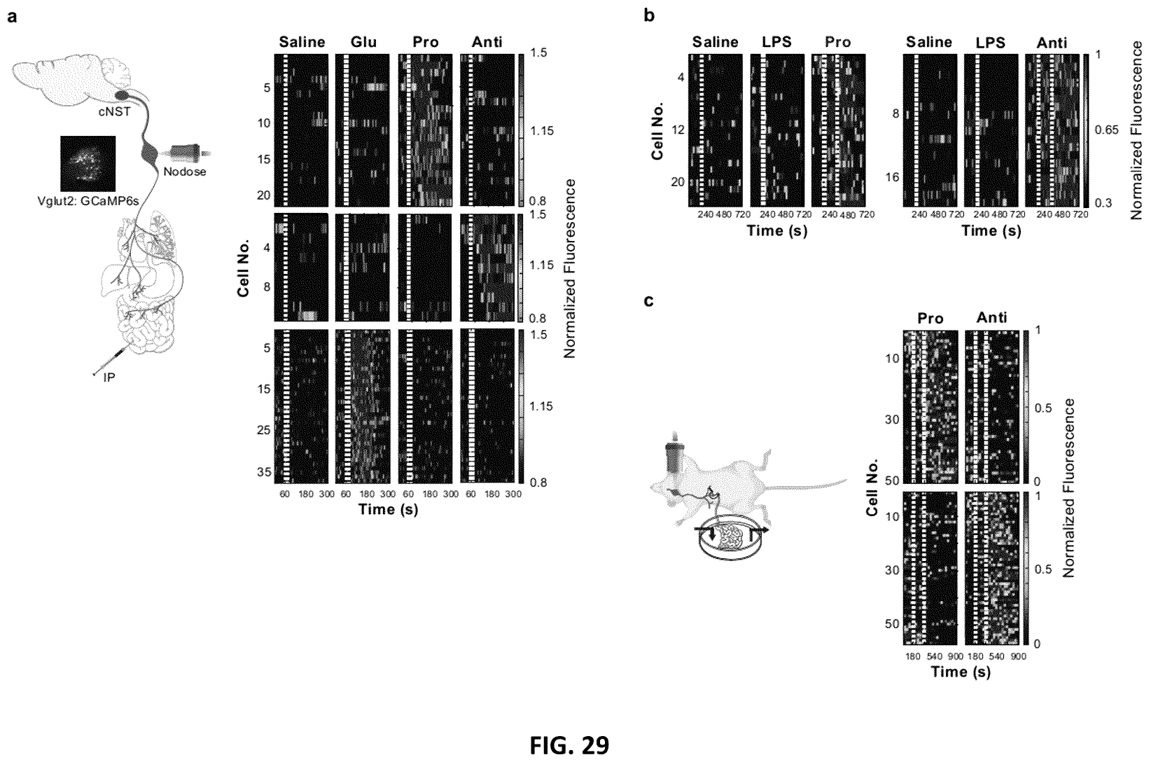

An in vivo calcium imaging platform was implemented to record immune-evoked neural activity in the nodose (vagal) ganglia where the cell bodies of vagal sensory neurons reside, while animals were challenged with different cytokines. The calcium indicator GCaMP6s was targeted to all vagal sensory neurons using a Vglut2-cre37 driver, and a one-photon functional imaging setup was used to record real-time vagal neuron responses to cytokine stimuli delivered intraperitoneally. As control, responses to LPS and to intestinal delivery of sugar, a stimulus known to activate the nutrient-sensing, gut-brain axis via a specific population of vagal neurons, were also imaged. The results showed that anti-inflammatory and pro-inflammatory cytokines activate two discrete non-overlapping populations of vagal sensory neurons, each accounting for a small fraction of all nodose ganglion neurons (FIG. 29 Panel a). These do not overlap with the sugar-sensing vagal neurons (FIG. 29 Panel a, bottom). Importantly, LPS does not directly activate vagal neurons (FIG. 29 Panel b).

Because the delivery of cytokines via intraperitoneal injections limits the ability to examine responses across repeat trials in the same animal, an in vivo preparation was implemented that enables repeated perfusion of cytokines over time. Since the small intestines are a major substrate of vagal innervation, and house a vast reservoir of immune cells capable of releasing cytokines in response to LPS stimulation, this may provide an effective strategy. The results demonstrated reproducible vagal responses to cytokine stimulation (FIGS. 29 (Panel c) and 41-42), thus substantiating that cytokines themselves function as an immune mediator in the body-brain axis, with the vagal neurons functioning as the conduit transmitting the inflammatory information to the cNST. Injection of cytokines activate the cNST DBH neurons (FIG. 39 Panels c and d), and activating the selective vagal neurons modulates the immune response, much like activating the cNST target neurons (see below).

### Example 7

### Vagal Neurons Respond to Inflammatory Signals

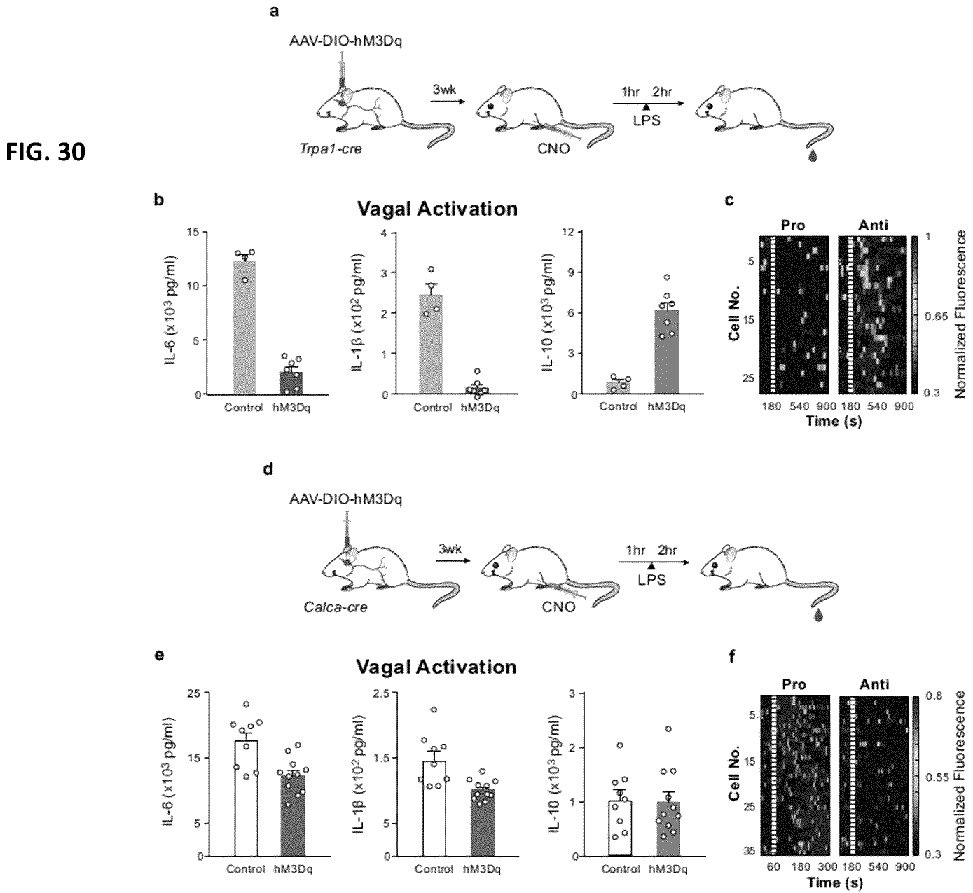

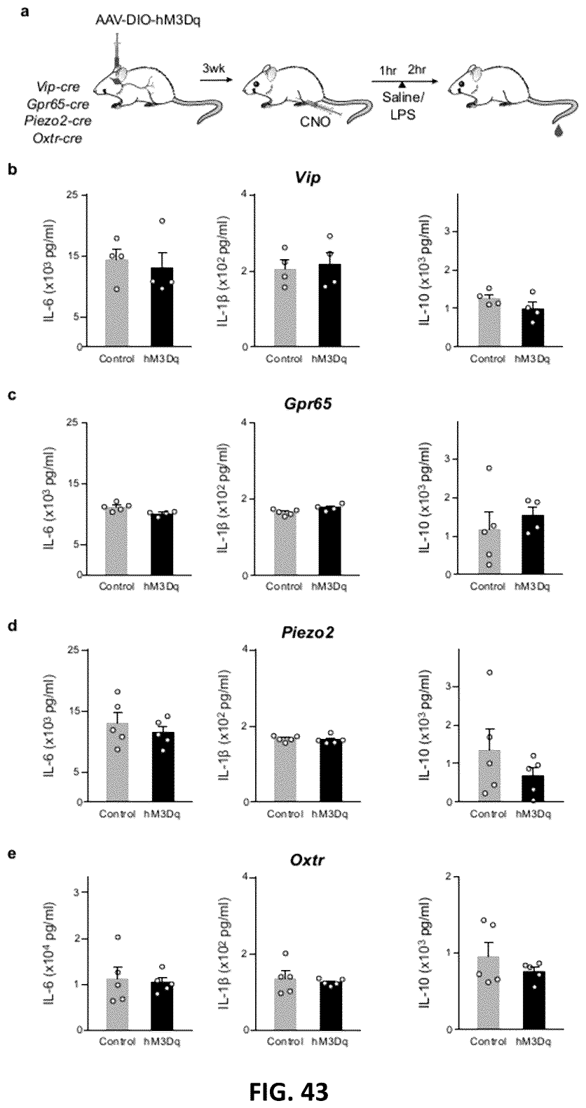

Because of the significance of suppressing an inflammatory state by modulating brain-body signals, identifying vagal neurons mediating anti-inflammatory responses was the first focus. The strategy was to use the scRNA-seq cell atlas of the nodose ganglion to target excitatory DREADDs to different populations, and assess the impact of activation on LPS-induced immune responses. To ensure that only vagal neurons are activated in these experiments, the AAV-DIO-hM3Dq (DREADD) virus was directly injected bilaterally into the nodose ganglia of the various cre-reporter mouse lines (FIGS. 30 and 43). The results showed that activating the Transient Receptor Potential Ankyrin 1 (TRPA1)-expressing vagal neurons dramatically enhances the anti-inflammatory response, while severely suppressing the levels of pro-inflammatory cytokines (FIG. 30 Panels a and b). Indeed, a more than 80% decrease in the circulating levels of pro-inflammatory cytokines, and a nearly 6-fold increase in the levels of IL-10 was observed.

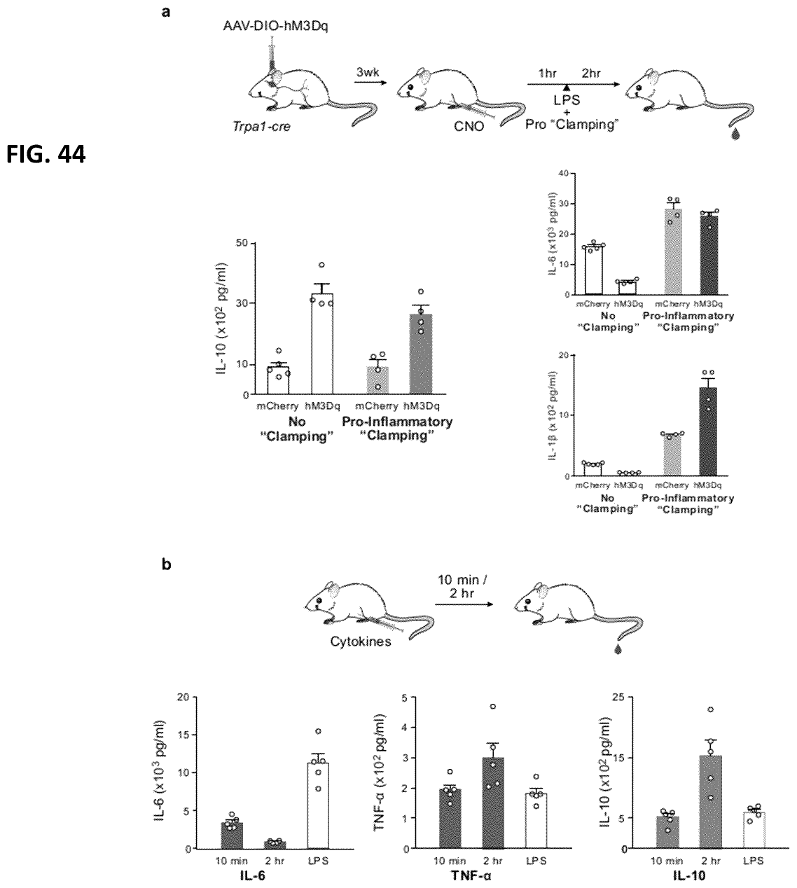

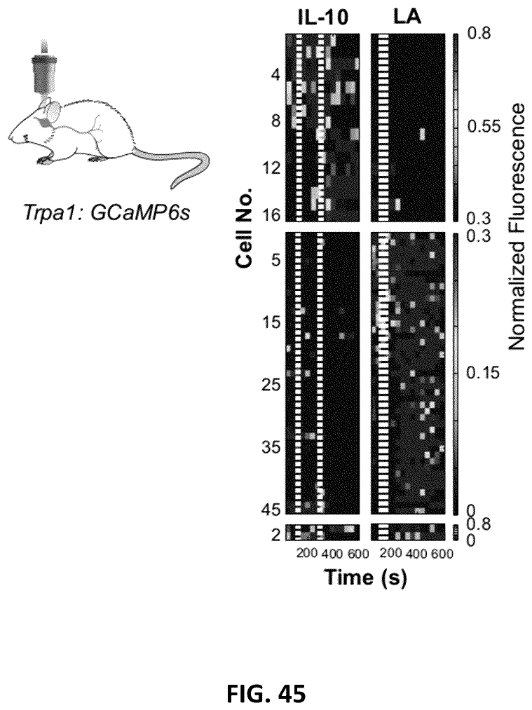

A “clamping-like” experiment that artificially maintains pro-inflammatory cytokines at high levels was performed and the anti-inflammatory response was examined when activating the TRPA1 neurons. The results demonstrated that despite persistently high levels of pro-inflammatory cytokines, IL-10 is still dramatically enhanced in response to TRPA1-neuron activation (FIG. 44 Panel a).

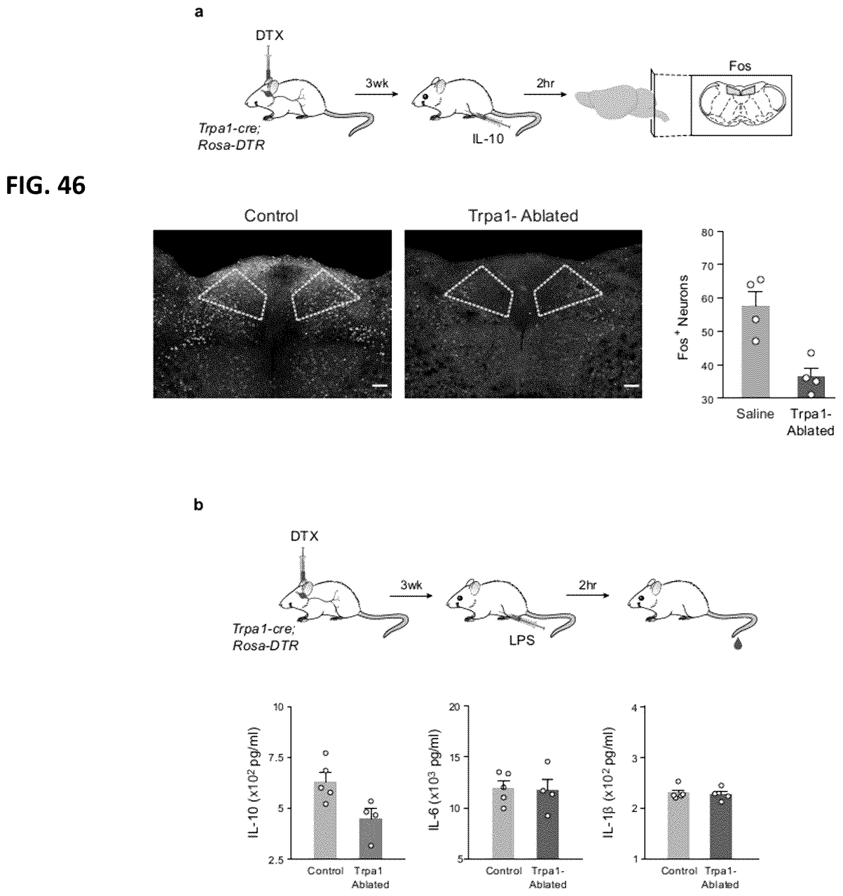

To define the response properties of the TRPA1-expressing vagal neurons, expression of GCaMP6s was targeted, and responses when the animals were challenged with anti-inflammatory or pro-inflammatory cytokines were imaged. IL-10, but not pro-inflammatory cytokines (IL-1β, IL-6) activated the TRPA1-expressing vagal neurons (FIGS. 30 (Panel c) and 45). Given these results, removing the TRPA1-vagal neurons from this circuit could prevent the transfer of anti-inflammatory signals to the brain. TRPA-1 expressing vagal neurons were genetically ablated by targeting the Diphtheria toxin receptor, and then the animals were challenged with IL-10 or LPS. Indeed, the results demonstrated that the cNST is now very poorly activated in response to IL-10 injection (FIG. 46 Panel a), and more importantly, the anti-inflammatory response is severely truncated; IL-10 levels are only about 50% of what is observed in control animals after LPS stimulation, with no effect on the pro-inflammatory response (FIG. 46 Panel b). These results reveal TRPA1-expressing vagal neuron as a conduit for relaying anti-inflammatory signals via the body-brain axis to reinforce the anti-inflammatory state.

The experiments showed that Calcitonin Related Polypeptide Alpha (CALCA)-expressing neurons in the vagal ganglia responded selectively to pro-inflammatory stimuli (FIG. 30 Panel f), and their chemogenetic activation significantly altered the levels of circulating pro-inflammatory cytokines (FIG. 30 Panels d and e).

### Example 8

### A Vagal-cNST Body-Brain Circuit

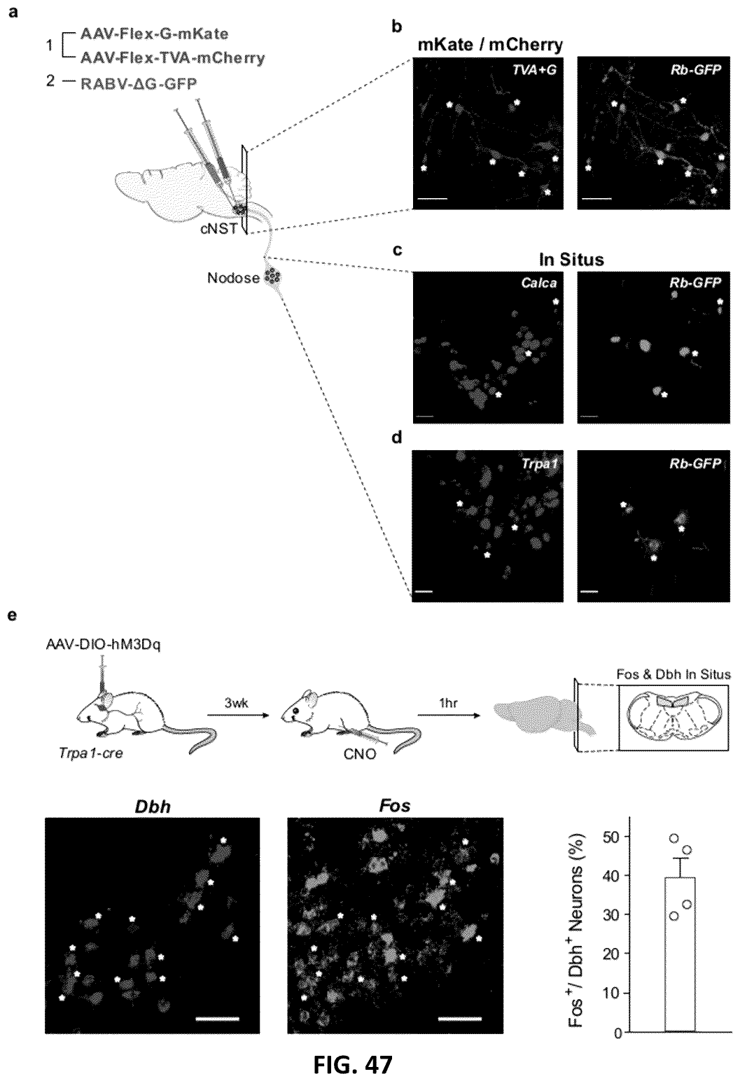

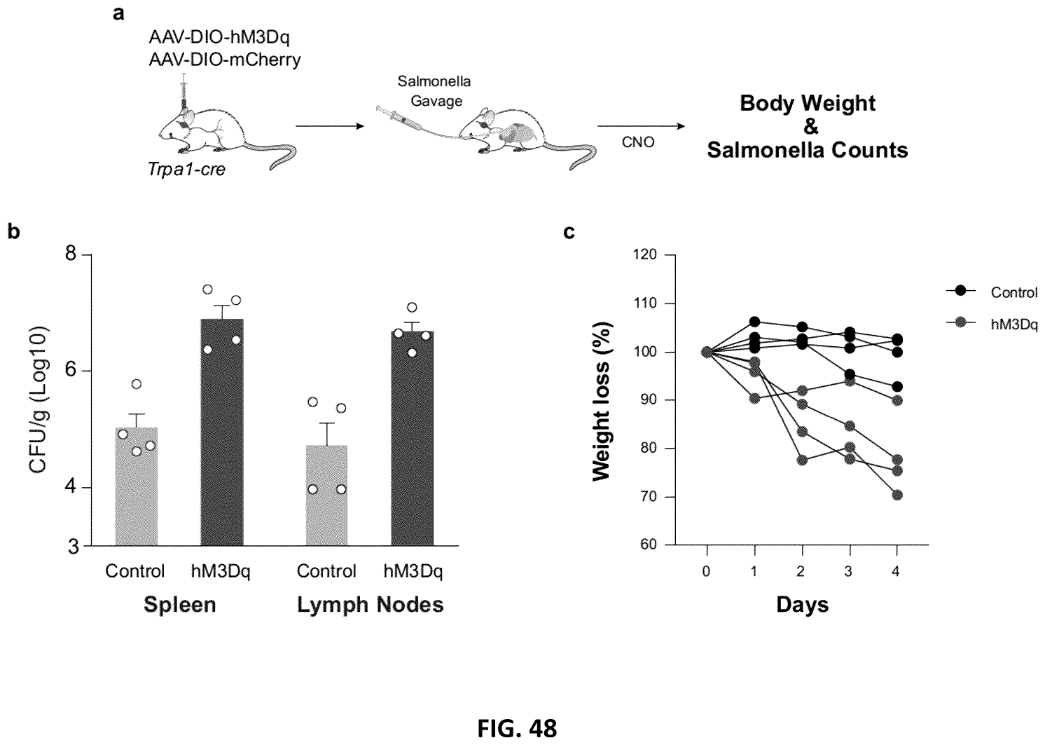

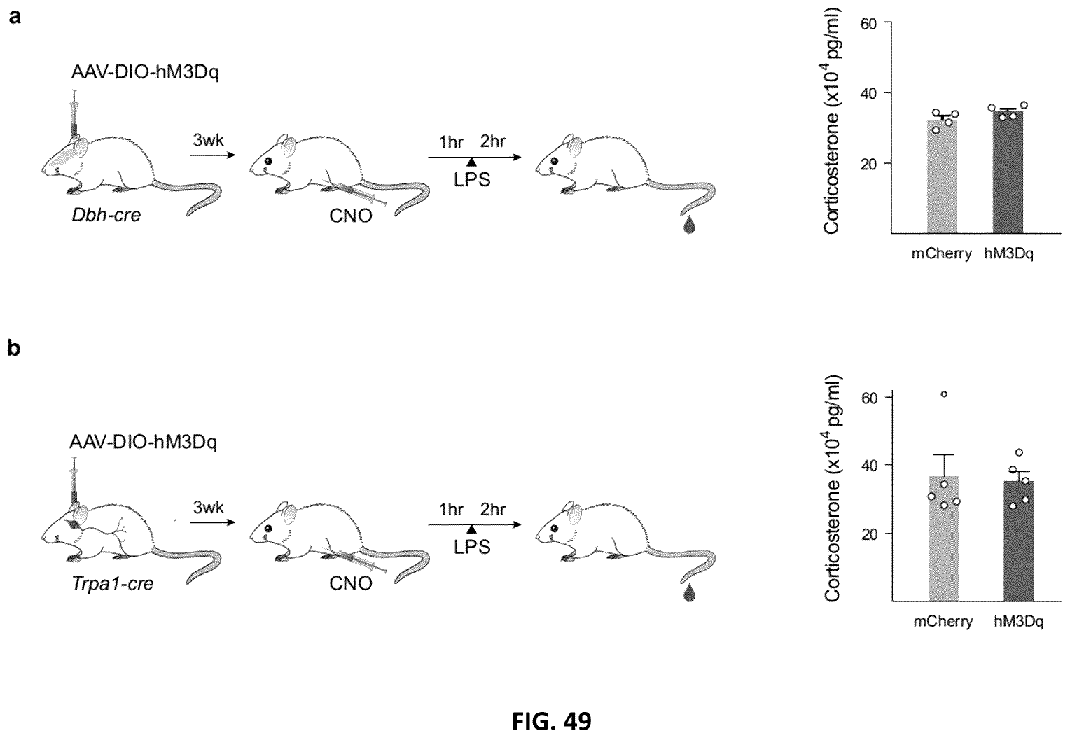

To demonstrate that the cNST DBH-expressing neurons receive direct input from the vagal ganglion neurons carrying the anti-inflammatory (expressing TRPA1) and pro-inflammatory (CALCA) signals, a Cre-dependent monosynaptic retrograde viral reporter system was used. In essence, the cNST of DBH-cre animals was infected with adeno-associated viruses (AAV) carrying a Cre-dependent glycoprotein coat and a surface receptor for a transsynaptic reporter. The DBH neurons harboring the viral receptor and G-protein were then infected with a retrograde rabies reporter (RABV-dsRed), and it was examined whether they receive input from TRPA1 and CALCA vagal ganglion neurons. The results shown in FIG. 47 panels a-d demonstrate transfer of the rabies reporter from the cNST to the vagal TRPA1 and CALCA neurons, confirming the monosynaptic connections between the immune responding neurons in the vagal ganglia and DBH neurons in the cNST. Next, the excitatory DREADD were targeted to TRPA1 vagal neurons, and their stimulation indeed robustly activated DBH neurons in the cNST (FIG. 47 Panel e).

Together, these results uncovered two lines of signaling from the vagal ganglia to the brain. One line (TRPA1), carries anti-inflammatory signals and acts on cNST neurons to enhance the anti-inflammatory response (for example, by positive feedback onto immune cells releasing anti-inflammatory cytokines), and helps suppress the pro-inflammatory state. The other (CALCA neurons), responds to pro-inflammatory signals and helps tune down the pro-inflammatory response (for example, by negative feedback onto immune cells releasing pro-inflammatory cytokines).

Activation of other vagal populations did not significantly impact the LPS-induced inflammatory responses (FIG. 43), further illustrating the specificity of this body-brain circuit.

### Example 9

### Restoring Immune Balance

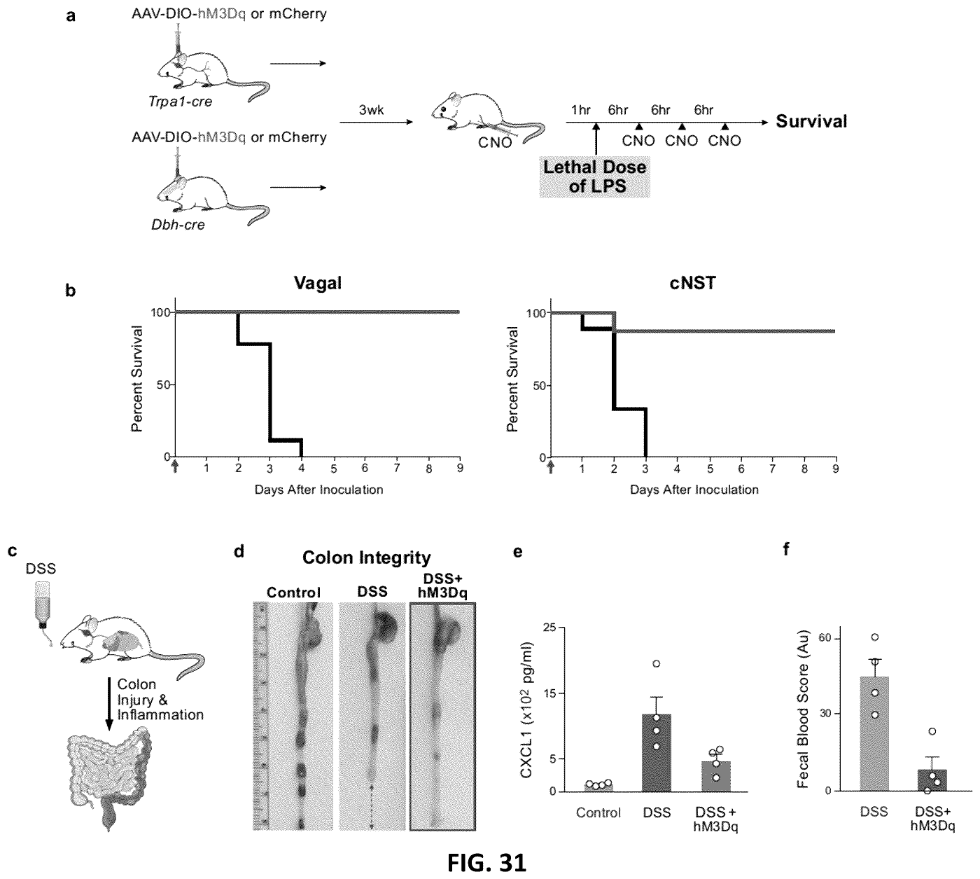

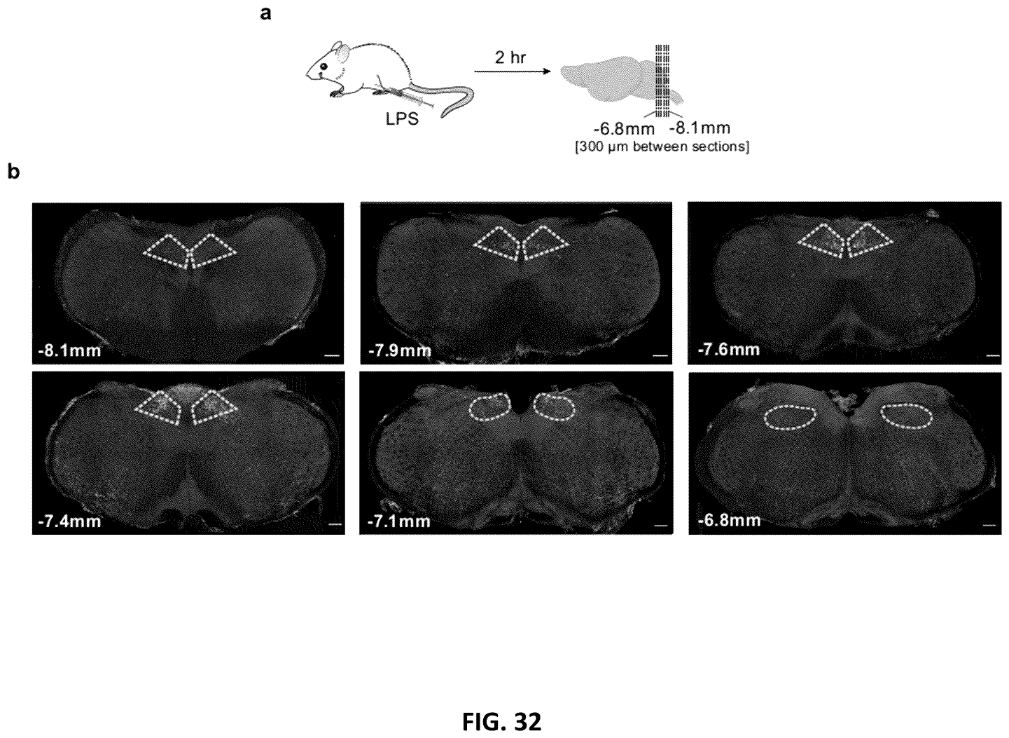

Control mice were injected with lethal doses of LPS (e.g., overwhelming the natural innate response), and the same injections were performed in animals where this circuit had been chemogenetically activated by targeted expression of excitatory DREADD to the TRPA1 vagal neurons (FIG. 31 Panel a). In parallel, the DBH-expressing neurons in the cNST were also targeted. Remarkably, chemogenetic activation of either of these neuronal populations in this immune-modulatory circuit is sufficient to dramatically transform the survival of these animals to an otherwise lethal dose of LPS: ˜90% of the mice are now alive after such intense immune challenge (FIG. 31 Panel b).

Finally, a mouse model of ulcerative colitis (DSS-induced intestinal inflammation) was used to examine if activation of this immune-modulatory circuit can prevent the dramatic loss of colon integrity, increase of pro-inflammatory cytokines, and high levels of fecal blood observed in this model of colon injury and inflammation. Control mice, and animals where the TRPA1 vagal neurons had been chemogenetically activated by targeted expression of excitatory DREADD, were exposed to DSS for 7 days; sufficient for the development of the severe pathologies triggered by DSS treatment. DSS-treated control animals exhibited dramatic damage to the distal colon, showed significant occult stool blood, and have a major increase in the levels of pro-inflammatory cytokines (FIG. 31 Panels c-f). By contrast, chemogenetic activation of the TRPA1 vagal neurons protected animals from all three pathological conditions (FIG. 31 Panels c-f, hM3Dq animals).

### Materials and Methods

### Animals

All procedures were performed in accordance with the U.S. National Institutes of Health (NIH) guidelines for the care and use of laboratory animals, and were approved by the Columbia University Institutional Animal Care and Use Committee. Mice both male and female and at least 7 weeks of age were used in the study. C56BL/6J (JAX 000664); Myd88−/−69 (JAX 009088); TRAP240 (JAX 030323); Dbh-cre46 (JAX 033951); Vip-IRES-cre70 (JAX 010908); Gpr65-IRES-cre71 (JAX 029282); Piezo2-cre72 (JAX 027719); Oxtr-IRES-cre73 (JAX 030543); Vglut2-IRES-cre37 (JAX 028863); Vgat-IRES-cre37 (JAX 016962); Ai941 (JAX 007909); Ai9674 (JAX 028866); Ai16275 (JAX 031562); Rosa-iDTR76 (JAX 007900) were obtained from the Jackson Laboratory. Trpa1-IRES-cre6 was generated in the Zuker lab. Calca-cre53 mice were a generous gift of Richard Palmiter.

### Fos Stimulation and Histology

Mice housed in their home cages were injected intraperitoneally with lipopolysaccharide (LPS, 50 μg kg-1, Cell Signaling Technology, #14011), lipoteichoic acid (LTA, 1 mg kg-1 Sigma, #L2512), Flagellin (20 μg kg-1, Sigma Aldrich #SRP8029), Profilin (20 μg kg-1, Sigma Aldrich, #SRP8050), Zymosan (2.5 mg kg-1, Sigma Aldrich #Z4250), IL-10 (100 μg kg-1, BioLegend, #575804), a cocktail of IL-6 (100 μg kg-1, BioLegend #575706), IL-1β (100 μg kg-1, R&D, #401-ML) and TNF-α (100 μg kg-1, R&D, #410-MT), or saline control (0.9% NaCl), then 2 h later, perfused transcardially with PBS followed by 4% paraformaldehyde. Brains were dissected, fixed in 4% PFA overnight at 4° C., and then sliced coronally at 100 μm thickness. The brain sections were permeabilized and blocked with 10% normal donkey serum (EMD Millipore, #S30) in PBS containing 0.3% Triton X-100. Sections were incubated with an anti-Fos primary antibody (SYSY, #226004 guinea pig, diluted 1:5,000) at 4° C. overnight, followed by labeling with a secondary antibody (Alexa Fluor 647-conjugated donkey anti-guinea pig, Jackson ImmunoResearch, #706605148) at room temperature for 2 h. For double RNA in situ hybridization, fixed frozen nodose ganglia or brains were sectioned at 16 μm thickness and processed for mRNA detection using the RNAscope Fluorescent Multiplex Kit (Advanced Cell Diagnostics) following the manufacturer's instructions. The following RNAscope probes were used: Fos (#316921-C2), Dbh (#464621-C1), Trpa1 (#400211-C3) and Calca (#578771-C2), GFP (#400281-C1). Images were acquired using an Olympus FluoView 1000 confocal microscope. Quantification of fluorescent signals was carried out by manually counting the number of positive neurons.

### Stereotaxic Surgery

All stereotaxic surgery procedures were carried out using aseptic technique. Mice were anesthetized with a mixture of ketamine and xylazine (100/10 mg kg-1, intraperitoneally) and then positioned on a custom-built stereotaxic frame equipped with a closed-loop heating system to maintain their body temperature. The viral constructs were injected into the cNST through a small craniotomy. The injection coordinates (based on Paxinos stereotaxic coordinates) for virus delivery in the cNST were as follows: caudal 7.5 mm, lateral ±0.3 mm, ventral 3.7-4 mm, all relative to Bregma. In chemogenetic experiments, TRAP2, Dbh-cre, Vglut2-cre and Vgat-cre mice received bilateral injections of 200 nl of AAV9-Syn-DIO-hM3Dq (Addgene, #44361-AAV9) and 300 nl of AAV9-Syn-DIO-hM4Di (Addgene, #44362-AAV9) in the cNST. Equivalent volumes of AAV9-Syn-DIO-mCherry (Addgene, #50459-AAV9) were injected as controls. For fiber photometry experiments, Vglut2-cre mice were unilaterally injected with 100 nl of AAV9-Syn-Flex-GCaMP6s (Addgene, #100845-AAV9) in the cNST, and an optical fiber (400 μm core, 0.48 NA, Doric Lenses) was implanted 50-100 μm above the GCaMP virus injection site.

### Fiber Photometry and Subdiaphragmatic Vagotomy

Photometry experiments were conducted at least 14 days after the stereotaxic viral injection and fiber implantation (see the section on stereotaxic surgery for details). Prior to the experiments, mice were acclimated to the recording chamber for 1 h per day over 3 consecutive days. On the 4th and 5th day, mice were recorded for the bulk GCaMP responses to saline and LPS (0.5 mg kg-1), respectively, in a 5 h recording session. Saline and LPS were intraperitoneally injected 15 min after the onset of recording. Real-time population-level GCaMP fluorescence signals were detected, amplified and recorded using a RZ5P fiber photometry system with Synapse software (Tucker Davis Technologies). The collected data were downsampled, detrended and smoothed by custom MATLAB code. The calcium transients were identified as described previously (Barretto, R. P. et al. Nature 517, 373-376, (2015); Rousseeuw, P. J. C., C. J. Am. Stat. Assoc. 88, 1273-1283, (1993); Jin, H., Fishman, Z. H., Ye, M., Wang, L. & Zuker, C. S. Cell 184, 257-271 e216, (2021)) and the area under the curve (AUC) was calculated by integrating fluorescence signal under identified calcium transients.

To assess the necessity of the vagus nerve in the cNST responses to LPS, a separate group of Vglut2-cre mice received bilateral subdiaphragmatic vagotomy as previously described6,39, following the injection of GCaMP virus and the implantation of the fiber in the cNST. Mice were anesthetized with ketamine and xylazine (100/10 mg kg-1, intraperitoneally). The stomach and esophagus were carefully exposed to avoid any damage to blood vessels or the liver. The dorsal and ventral branches of the vagus nerve along the subdiaphragmatic esophagus were then exposed, and the right and left vagus nerve were transected. The abdominal muscle layer and skin were closed with sutures. Following the vagotomy procedure, the mice were given two weeks to recover before fiber photometry recordings. The expression of GCAMP and placement of optic fibers were histologically verified at the termination of the experiments.

### Genetic Access to LPS-Activated Neurons in the Brain

The TRAP strategy was used in TRAP2 mice to gain genetic access to LPS-activated neurons in the cNST. The AAV-injected TRAP2 mice (2-3 weeks after viral injection), or TRAP2; Ai9 mice, were first habituated to intraperitoneal injections by daily injection of 100 μl saline for 5 days. After habituation, LPS (50 μg kg-1) was given intraperitoneally, then 90 mins later, 4-hydroxytamoxifen (4-OHT, 20 mg kg-1, Sigma, #H6278) was administered. Mice were used for experiments a minimum of 4 weeks after this TRAP protocol; this extended waiting time is crucial to restore sensitivity to LPS after the initial LPS-induced TRAPing.

### Chemogenetic Manipulation Experiments and Measurement of Cytokines

Following bilateral injection with AAV9-Syn-DIO-hM3Dq in the cNST of Dbh-cre, Vglut2-cre or Vgat-cre mice, or in the nodose ganglion of Trpa1-cre, Calca-cre, Vip-cre, Gpr65-cre, Piezo2-cre, and Oxtr-cre mice, the animals were allowed to recover for a minimum of three weeks prior to treatment with CNO (Enzo life sciences #BML-NS105). When using TRAP2 animals, at least 4 weeks elapsed between TRAPing and CNO treatment. Two doses (2 mg kg-1 and 1 mg kg-1) of CNO were given intraperitoneally at 12 h and 1 h prior to saline or LPS stimulation. Two hours after the intraperitoneal injection of saline or LPS (0.1 mg kg-1), blood samples were collected from either the submandibular or the tail vein. Cytokines in the blood were measured using commercially available ELISA kits (R&D), following manufacturer's instructions. Saline and LPS experiments were conducted on the same cohort of mice but at least 7 days apart.

To examine LPS-induced cytokine responses over time, wild-type (C56BL/6J) mice were injected IP with saline or LPS, and peripheral blood samples were collected at 0, 2 hours, 4 hours, and 6 hours post-stimulation.

To measure circulating cytokine levels following administration of exogenous cytokines, mice were injected IP with IL-6 (100 μg kg-1), TNF-α (100 μg kg-1) or IL-10 (100 μg kg-1), and peripheral blood samples were harvested at 2 hours post-injection.

To “clamp” pro-inflammatory cytokine levels, a cocktail of IL-6 (300 μg kg-1), IL-1β (15 μg kg-1), and TNF-α (30 μg kg-1) was injected with LPS.

Saporin-Ablation of DBH cNST Neurons

Saporin-mediated targeted ablation is a highly effective method to kill DBH-neurons. The cNST of mice were bilaterally injected with an anti-DBH-saporin conjugate (20 ng per side, Advanced Targeting Systems, #IT-03), and after 2-3 weeks recovery, animals were stimulated with LPS (0.1 mg kg-1) intraperitoneally. One hour following the LPS injections, blood samples were collected for measuring cytokines in the control and the anti-DBH-saporin treated animals.

Single-Cell RNA Sequencing of cNST and LPS-TRAPed Cells

To perform scRNA-seq on the entire cNST, single cells were isolated from the cNST as previously described (Pool, A. H. et al. Nature 588, 112-117, (2020)) with the following modifications. Briefly, mice were anaesthetized with isoflurane and transcardially perfused with ice-cold carbogenated (95% O₂, 5% CO₂) NMDG-HEPES-ACSF (93 mM NMDG, 2.5 mM KCl, 1.2 mM NaH₂PO₄, 30 mM NaHCO₃, 20 mM HEPES, 25 mM glucose, 10 mM MgSO₄, 1 mM CaCl₂, 1 mM kynurenic-acid Na salt, 5 mM Na-ascorbate, 2 mM Thiourea, 3 mM Na-pyruvate, pH 7.4). The brainstems were rapidly extracted and sliced into 300 m sections containing the cNST using a vibratome (Leica, #VT-1000S) in ice-cold NMDG-HEPES-ACSF solution with continuous carbogenation. The cNSTs were dissected, pooled (from 5 animals), and digested in Trehalose-HEPES-ACSF (92 mM NaCl, 2.5 mM KCl, 1.25 mM NaH₂PO₄, 30 mM NaHCO₃, 20 mM HEPES, 25 mM glucose, 2 mM MgSO₄, 2 mM CaCl₂, 1 mM kynurenic-acid Na salt, 2.5 wt/vol trehalose, pH 7.4) containing Papain (20 U ml-1, Worthington, #LK003150) and DNase I (25 U ml-1) at 35° C. for approximately one hour. Using Pasteur pipettes with progressively narrowing tip diameters, the tissue was triturated in DNase I-containing (25 U ml-1) Trehalose-HEPES-ACSF solution to form single-cell suspension. The dissociated cells were passed through 40 μm filter and resuspended in Resuspension-ACSF (117 mM NaCl, 2.5 mM KCl, 1.25 mM NaH₂PO₄, 30 mM NaHCO₃, 20 mM HEPES, 25 mM glucose, 1 mM MgSO₄, 2 mM CaCl₂, 1 mM kynurenic-acid Na salt, 0.05% BSA, pH 7.4). The resulting cell suspension was processed by the Columbia Genome Core to encapsulate and barcode individual cells using the 10× Genomics Chromium system.

For sequencing LPS-TRAPed cells, TRAP2 was used; Ai9 mice that were TRAP-labeled with tdTomato in response to LPS. Cells from the cNST were isolated as described above. The cell suspension was stained with DRAQ5 (Thermo Scientific, #62254) and Calcein Violet (Thermo Scientific, #C34858) to label viable cells, prior to FACS. A total of 288 tdTomato+ LPS TRAPped cells and 96 tdTomato-cells were sorted into 96-well plates pre-loaded with cell lysis buffer containing 0.1% TritonX-100, SuperaseIN (Ambion, #AM2694), 1 mM dNTP and 1 M capture primer (i.e. bar-coding). cDNA was synthesized using Maxima Reverse Transcriptase (Thermo Scientific, #EP0753) according to manufacturer's instruction. cDNA from all the wells/cells was combined, followed by clean-up using Silane beads (Thermo Scientific #37002D). Pooled cDNA was amplified using Kapa HotStart Mix with SMART PCR primer (0.2 M), and then purified using AMPureXP beads (Beckman Coulter Life Sciences #A63880). 0.6 ng of cDNA was used as input to prepare libraries using Nextera XT kit (Illumina #FC-131-1024). The resulting libraries were sequenced on an Illumina sequencer.

scRNA-seq Data Analysis

Illumina sequencing reads were mapped to the mouse genome using the CellRanger pipeline with the default parameters. Analysis of scRNA-seq data, including the generation of cell clusters and identification of neuronal cluster markers, were performed using custom R code developed following Seurat online instructions and vignettes. Genes that were expressed in fewer than 10 cells in the cNST-seq dataset, and in fewer than 3 cells in the TRAP2-seq dataset were removed. Additionally, cells with low-depth sequencing (fewer than 2000 genes in the cNST-seq dataset) were removed. To integrate datasets from cNST-seq and TRAP2-seq, the standard scRNA-seq integration procedure as outlined by Seurat (satijalab.org/seurat/) was employed. Briefly, each dataset was first normalized and then the Seurat “FindVariableGenes” routine was used to identify 2000 variable genes from each sample. Then, a common set of variable features were determined by Seurat “SelectIntegrationFeatures” to merge samples. Finally, the first 25 principal components (PCs) were used for generating cell types utilizing Seurat's “FindClusters”.

### Nodose Ganglion Injection Experiments

The injection of AAV to nodose ganglion was performed as described previously. In brief, Cre-expressing mice (Trpa1-cre, Calca-cre, Vip-cre, Piezo2-cre, Gpr65-cre, and Oxtr-cre) were anaesthetized with intraperitoneal administration of ketamine and xylazine (100/10 mg kg-1). The skin under the neck was shaved and an incision (˜1.5 cm) in the midline was made. The trachea and surrounding muscles were gently retracted to expose the nodose ganglia. A mixture of Fast Green (Sigma, #F7252) and AAV carrying the Cre-dependent excitatory DREADD (AAV9-Syn-DIO-hM3Dq) or AAV9-Syn-DIO-mCherry (control) was injected to both left and right ganglia using a 30° beveled glass pipette (Clunbury Scientific). The injection volume per ganglion was 300 nl. For experiments ablating TRPA1 neurons, TRPA1-cre mice were crossed to Rosa-DTR mice, and vagal ganglia were bilaterally injected with control PBS alone or PBS containing 2 ng DTX (200-nL total volume; Sigma Aldrich #D0564)₅₂. At the end of surgery, the skin incision was closed using 5-0 absorbable sutures (CP medical, #421A). Following the procedure, mice were allowed to recover for a minimum of 21 days prior to testing. The viral expression and ablation efficiency was histologically confirmed by examining the nodose ganglia extracted from all tested animals; mice with insufficient viral expression, mis-targeting of viral injection, or unsuccessful ablation were removed from data analysis.

### Vagal Calcium Imaging

Calcium imaging of the nodose ganglion was conducted as described previously (Li, M. et al. Nature 610, 722-730, (2022); Tan, H. E. et al. Nature 580, 511-516, (2020)). For imaging in response to intragastric delivery of glucose (or linoleic acid), and IP injections of saline control and cytokines, a typical recording session consisted of: 1st: saline; 2nd: one of the three pro-inflammatory cytokines (TNF-α, IL-1β or IL-6); 3rd: anti-inflammatory cytokine (IL-10); 4th and 5th: two trials with glucose (or linoleic acid); each trial was 5 min. Cytokines were injected 1 min after the onset of the recording. Glucose (500 mM) and linoleic acid (10%) was delivered intragastrically. For all experiments 100 μg kg-1 of each cytokine was used. To deliver cytokines extraintestinally, a segment of the intestine was placed in a custom-made perfusion chamber while still keeping it connected to the remainder of the gastrointestinal tract (no carbogenation). Each recording session included six interleaved trials, with 2 trials for each stimulus. Trials were 15 mins long, and consisted of a 180-s baseline (saline), a 180-s cytokine or control stimulus, and a 9-min washout (saline) period. The flow rates were maintained at around 600 μl min-1 throughout the experiment to minimize mechanical responses that may occur during the transition between trials. Cytokines were dissolved in saline at the concentration of 1 μg ml-1. During the entire perfusion session, all of the solutions were maintained at 37° C.

### Calcium Imaging Data Collection and Analysis

Imaging data was acquired exactly as previously described (Li, M. et al. Nature 610, 722-730, (2022); Tan, H. E. et al. Nature 580, 511-516, (2020)). Neuronal activity was analyzed for significant stimulus-evoked responses as described. The baseline distribution of deviations was first computed from the median for each cell throughout the entire experiment using periods prior to the stimulus delivery. Subsequently, this baseline was utilized to derive a modified z-score by subtracting the median and dividing by the median absolute deviation. Trials with an average modified z-score above 1.6 for the 180s (stimuli delivered via IP) or 480s (stimuli delivered via perfusion) following the initiation of stimulation were classified as responding trials (all responders had minimal peak amplitudes of 1% ΔF/F). Z-scores from responders were normalized across stimuli to generate heat maps of normalized fluorescence traces.

### Mapping Vagal-to-cNST Circuit

For monosynaptic retrograde tracing experiments, the cNST of Dbh-Cre animals were first injected with a 1:1 mixture of AAV1-DIO-TVA-mcherry and AAV1-DIO-G(N2C)-mKate followed by a second injection of EnvA-pseudotyped G-deleted rabies virus carrying GFP reporter (RABV-N2C(ΔG)-GFP-EnvA) 3 weeks later. Two weeks after RABV infection, the animals were sacrificed to identify, and examine presynaptic neurons in the nodose ganglion by RNA in situ hybridization. To determine if DBH neurons are activated by stimulation of TRPA1 vagal neurons, AAVs carrying the Cre-dependent excitatory DREADD (AAV9-Syn-DIO-hM3Dq) were injected into the nodose ganglia of Trpa1-cre mice (see “Nodose ganglion injection experiments” section). Following injection, the animals were allowed to recover for a minimum of three weeks prior to TRPA1 vagal neuron activation with CNO. CNO (5 mg kg-1) was injected intraperitoneally, and 1 hr later, mice were euthanized to examine co-expression of Fos and Dbh in the cNST by in situ hybridization.

### Modulation of Survival in LPS-Induced Endotoxemia Through Chemogenetic Activation of the Vagal-Brainstem Axis

After bilateral injection of AAV9-Syn-DIO-hM3Dq or AAV9-Syn-DIO-mCherry (control) in the cNST of Dbh-cre mice, and in the nodose ganglion of Trpa1-cre mice, animals were allowed to recover for a minimum of three weeks before the injection of LPS. CNO (5 mg kg-1) was intraperitoneally administered 1 h prior to a lethal dose of LPS (12.5 mg kg-1). Following the LPS challenge, CNO (5 mg kg-1) was administered every 6 hours for a total of 3 doses; survival was monitored every 6 hours.

### Dextran Sodium Sulfate (DSS)-Induced Colitis and Chemogenetic Activation of TRPA1 Vagal Neurons

Trpa1-cre mice were injected bilaterally in the nodose ganglia with AAV9-Syn-DIO-hM3Dq, or control AAV9-Syn-DIO-mCherry. Three weeks later, they were exposed to 3% DSS in the drinking water for 7 days. CNO (0.03 mg ml-1) was added to DSS solution of the experimental cohort to concomitantly activate TRPA1 neurons. To motivate mice to drink, 10 mM Acek was added to the drinking mix in both groups. Colon morphology was examined at the termination of the experiment; CXCL-1 levels were measured using Elisa (R&D). Fecal occult blood was monitored using Hemoccult Dispensapak Plus (Beckman Coulter #61130) according to the manufacturer's instruction.

Salmonella enterica serovar Typhimurium (STm) Infection and Chemogenetic Activation of TRPA1 Vagal Neurons

Trpa1-cre mice injected with AAV9-Syn-DIO-hM3Dq or AAV9-Syn-DIO-mCherry (control) in the nodose were allowed 3-4 weeks for virus for reporter expression, and then infected with 1˜2×10\hat{ }7 CFU of (STm, ATCC, #14028) through oral gavage. CNO (5 mg kg-1) was injected at 12 h intervals beginning 12 h prior to STm gavage, for a total of 8 injections over 4 days. As a proxy for the animal's health, body weight was monitored daily. At day 5 post infection, the tissues (spleen and mesenteric lymph nodes) were collected from the infected mice, homogenized for serial dilutions in PBS, and plated on LB agar; CFU were counted after overnight incubation of the plates at 37° C.

### Statistics

No statistical methods were used to predetermine sample size, and investigators were not blinded to group allocation. No method of randomization was used to determine how animals were allocated to experimental groups. Statistical methods used include Mann-Whitney U-test, Wilcoxon test, one-way ANOVA and Log-rank (Mantel-Cox) test, and are indicated for all figures. Analyses were performed in MATLAB, R and GraphPad Prism 8. Data are presented as mean±SEM.

Here, it is showed that cytokines themselves mediate the activation of the vagal-brain axis, and characterized the key neuronal elements and the logic of the circuit. Most unexpectedly, this body-brain circuit modulates not only pro-inflammatory, but also the anti-inflammatory response. Indeed, a population of vagal neurons was identified that respond to pro-inflammatory and a different one responding to anti-inflammatory signals that transfer inflammatory information from the body to neurons in the cNST. Targeting this circuit may provide exciting new strategies to modulate and manage immune disorders, including autoimmune diseases (e.g., rheumatoid arthritis), cytokine storm, toxic shock, and other hyperactive immune states, like those promoted by powerful new immunotherapies.

When ranges are used herein for physical properties, such as molecular weight, or chemical properties, such as chemical formulae, all combinations, and subcombinations of ranges for specific embodiments therein are intended to be included.

Those skilled in the art will appreciate that numerous changes and modifications can be made to the preferred embodiments of the methods and that such changes and modifications can be made without departing from the spirit of the methods. It is, therefore, intended that the appended claims cover all such equivalent variations as fall within the true spirit and scope of the methods.

1-14. (canceled)

15. A method of assessing the ability of a stimulus to regulate an immune response, comprising:
applying the stimulus to a subject;
measuring in the subject, in response to the stimulus, activation of dopamine beta-hydroxylase (Dbh)-positive neurons; and
identifying the stimulus as capable of regulating the immune response based on the activation of the Dbh-positive neurons.

16. The method of claim 15, further comprising measuring in the subject, in response to the stimulus, activation of Dbh-negative neurons, and comparing the activation by the stimulus of the Dbh-positive and Dbh-negative neurons.

17. The method of claim 15, wherein the Dbh-positive neurons are present in the subject's caudal nucleus of the solitary tract (cNST).

18. The method of claim 15, wherein applying the stimulus comprises applying electrical stimulation, magnetic stimulation, mechanical stimulation, or administering an agent to the subject.

19. The method of claim 18, wherein applying the stimulus comprises administering the agent to the subject.

20. (canceled)

21. The method of claim 19, wherein the agent is a small molecule compound, an antibody, a protein, or a nucleic acid.

22. The method of claim 15, wherein measuring the activation comprises detecting calcium response.

23. The method of claim 22, wherein the neurons comprise a calcium indicator.

24. The method of claim 23, wherein the calcium indicator is genetically encoded in the neurons.

25. The method of claim 23, wherein the calcium indicator is a fluorescent or luminescent calcium indicator.

26. (canceled)

27. The method of claim 23, wherein detecting calcium response comprises in vivo imaging of the calcium indicator.

28. The method of claim 15, wherein the subject is a mammal.

29. The method of claim 15, wherein the subject is a mouse.

30. A model animal for screening the ability of a stimulus to regulate an immune response, the animal comprising:
a genetically encoded calcium indicator expressed in glutamatergic neurons of the animal.

31. The model animal of claim 30, wherein the glutamatergic neurons are present in the caudal nucleus of the solitary tract (cNST) of the animal.

32. The model animal of claim 30, wherein the genetically encoded calcium indicator is expressed in vglut2-positive neurons.

33. The model animal of claim 30, wherein the genetically encoded calcium indicator is expressed in Dbh-positive neurons.

34. The model animal of claim 30, wherein the animal is a mammal.

35. (canceled)

36. The model animal of claim 30, wherein the calcium indicator is a is a fluorescent or luminescent calcium indicator.

37. (canceled)

38. A method of regulating an immune response in a subject in need thereof, comprising: applying a stimulus to the subject capable of selectively activating glutamatergic neurons in the subject's caudal nucleus of the solitary tract, wherein activation of the glutamatergic neurons suppresses an immune response in the subject.

39-49. (canceled)
